SEQUENCE LISTING (9 sequences)

SEQ ID NO: 1 - protein, 5 aa, Artificial Sequence
  from human Fab library
SYIMM

SEQ ID NO: 2 - protein, 17 aa, Artificial Sequence
  from human Fab library
SIYPSGGITFYADTVKG

SEQ ID NO: 3 - protein, 11 aa, Artificial Sequence
  from human Fab library
IKLGTVTTVDY

SEQ ID NO: 4 - protein, 14 aa, Artificial Sequence
  from human Fab library
TGTSSDVGGYNYVS

SEQ ID NO: 5 - protein, 7 aa, Artificial Sequence
  from human Fab library
DVSNRPS

SEQ ID NO: 6 - protein, 10 aa, Artificial Sequence
  from human Fab library
SSYTSSSTRV

SEQ ID NO: 7 - protein, 450 aa, Artificial Sequence
  from human Fab library
EVQLLESGGGLVQPGGSLRLSCAASGFTFSSYIMMWVRQAPGKGLEWVSSIYPSGGITFYADTVKGRFTISRDNSKNTLYLQMNSLRAEDTAVYYCARIKLGTVTTVDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 8 - protein, 449 aa, Artificial Sequence
  from human Fab library
EVQLLESGGGLVQPGGSLRLSCAASGFTFSSYIMMWVRQAPGKGLEWVSSIYPSGGITFYADTVKGRFTISRDNSKNTLYLQMNSLRAEDTAVYYCARIKLGTVTTVDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 9 - protein, 216 aa, Artificial Sequence
  from human Fab library
QSALTQPASVSGSPGQSITISCTGTSSDVGGYNYVSWYQQHPGKAPKLMIYDVSNRPSGVSNRFSGSKSGNTASLTISGLQAEDEADYYCSSYTSSSTRVFGTGTKVTVLGQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

(19) United States
(12) Patent Application Publication
(10) Pub. No.: US 2021/0002371 A1
(43) Pub. Date: Jan. 7, 2021

(54) COMBINATION OF AN ANTI-PD-L1 ANTIBODY AND IDO1 INHIBITOR FOR THE TREATMENT OF CANCER

(71) Applicants: Brian A. SHERER, Nashua, NH (US); CIaude GIMMI, Moehlin (CH); Jacques MOISAN, Acton, MA (US); Merck Patent GmbH, Darmstadt (DE); Pfizer Inc., New York, NY (US)

(72) Inventors: Brian A. SHERER, Nashua, NH (US); Claude GIMMI, Moehlin (CH); Jacques MOISAN, Acton, MA (US)

(21) Appl. No.: 16/955,477

(22) PCT Filed: Dec. 19, 2018
(86) PCT No.: PCT/US18/66372

§ 371 Date: Jun. 18, 2020

Related U.S. Application Data
(60) Provisional application No. 62/775,025, filed on Dec. 4, 2018.
(60) Provisional application No. 62/608,694, filed on Dec. 21, 2017.

(51) Int. Cl. C07K 16/28; G01N 33/50; A61K 31/4188; A61P 35/00

(52) U.S. Cl. CPC C07K 16/2827; G01N 33/5011; A61K 31/4188; C12Y 113/11052; G01N 2800/52; G01N 2333/90241; A61P 35/00

(57) ABSTRACT

The present invention relates to combination therapies useful for the treatment of cancer. In particular, the invention relates to a therapeutic combination which comprises an anti-PD-L1 antibody and an IDO1 inhibitor. The therapeutic combination is particularly intended for use in treating a subject having a cancer that tests positive for PD-L1 or IDO1 expression.

### FIELD OF INVENTION

The present invention relates to combination therapies useful for the treatment of cancer. In particular, the invention relates to a therapeutic combination which comprises an anti-PD-L1 antibody and an IDO1 inhibitor. The therapeutic combination is particularly intended for use in treating a subject having a cancer that tests positive for PD-L1 and/or IDO1 expression.

### BACKGROUND OF THE INVENTION

Cancer immune evasion is a major obstacle to effective anti-cancer immunotherapy. Two prominent pathways that cancers exploit to evade immune-mediated destruction are the programmed death ligand 1 (PD-L1)/programmed death 1 (PD-1) immune checkpoint pathway and the kynurenine (Kyn) metabolic pathway, in which the essential amino acid tryptophan (Trp) is converted to Kyn as the first, rate-limiting step towards NAD production.

The mechanism of co-stimulation of T-cells has gained significant therapeutic interest in recent years for its potential to enhance cell-based immune response. Costimulatory molecules expressed on antigen-presenting cells (APCs) promote and induce T-cells to promote clonal expansion, cytokine secretion and effector function. In the absence of co-stimulation, T-cells can become refractory to antigen stimulation, do not mount an effective immune response, and further may result in exhaustion or tolerance to foreign antigens (Lenschow et al. (1996) Ann. Rev. Immunol. 14: 233). Recently, it has been discovered that T cell dysfunction or anergy occurs concurrently with an induced and sustained expression of the inhibitory receptor, PD-1 polypeptide. The PD-1 receptor and PD-1 ligands 1 and 2 (PD-L1 and PD-L2, respectively) play integral roles in immune regulation. Expressed on activated T cells, PD-1 is activated by PD-L1 (also known as B7-H1) and PD-L2 expressed by stromal cells, tumor cells, or both, initiating T-cell death and localized immune suppression (Dong et al. (1999) Nat Med 5: 1365; Freeman et al. (2000) J Exp Med 192: 1027), potentially providing an immune-tolerant environment for tumor development and growth. Conversely, inhibition of this interaction can enhance local T-cell responses and mediate antitumor activity in nonclinical animal models (Iwai et al. (2002) PNAS USA 99: 12293). As a result, therapeutic targeting of PD-1 and other molecules which signal through interactions with PD-1, such as PD-L1 and PD-L2 are an area of intense interest.

PD-L1 is overexpressed in many cancers and is often associated with poor prognosis (Okazaki et al. (2007) Intern. Immun. 19(7): 813; Thompson et al. (2006) Cancer Res 66(7): 3381). Interestingly, the majority of tumor infiltrating T lymphocytes predominantly express PD-1, in contrast to T lymphocytes in normal tissues and peripheral blood T lymphocytes, indicating that up-regulation of PD-1 on tumor-reactive T cells can contribute to impaired anti-tumor immune responses (Ahmadzadeh et al. (2009) Blood 14(8): 1537). This may be due to exploitation of PD-L1 signaling mediated by PD-L1 expressing tumor cells interacting with PD-1 expressing T cells to result in attenuation of T cell activation and evasion of immune surveillance (Keir et al. (2008) Annu. Rev. Immunol. 26: 677). Therefore, inhibition of the PD-L1/PD-1 interaction may enhance CD8+ T cell-mediated killing of tumors.

The inhibition of PD-1 axis signaling through its direct ligands (e.g., PD-L1 or PD-L2) has been proposed as a means to enhance T cell immunity for the treatment of cancer (e.g., tumor immunity). Moreover, similar enhancements to T cell immunity have been observed by inhibiting the binding of PD-L1 to the binding partner B7-1. Furthermore, combining inhibition of PD-1 signaling with other pathways would further optimize therapeutic properties (see e.g., WO 2016/205277 or WO 2016/032927).

The oxygenase indoleamine 2,3-dioxygenase (IDO1) or TRP-2,3-dioxygenase (TDO2) is responsible for the extra-hepatic conversion of Trp to N-formyl-kynurenine, its primary bioactive metabolite, as a rate-limiting first step of Trp metabolism. N-formyl-kynurenine is a precursor of a variety of bioactive molecules called kynurenines that have immunomodulatory properties (Schwarcz et al. (2012) Nat Rev Neurosci. 13(7): 465).

IDO1 is an inducible enzyme that has a primary role in immune cell modulation. The reduction of Trp levels and increase in the pool of kynurenines cause inhibition of effector immune cells and promote adaptive immune suppression through induction and maintenance of regulatory T cells (Tregs) (Munn (2012) Front Biosci. 4: 734).

Increased turnover of Trp to kynurenines by IDO1 has been observed in a number of disorders linked to activation of the immune system, e.g. infection, malignancy, autoimmune diseases, trauma and AIDS (Johnson and Munn (2012) Immunol Invest 41(6-7): 765). Additional studies in these indications have shown that induction of IDO1 results in suppression of T-cell responses and promotion of tolerance. In cancer, for example, a large body of evidence suggests that IDO1 upregulation serves as a mechanism in tumor cells to escape immune surveillance. IDO1 is expressed widely in solid tumors (Uyttenhove et al. (2003) Nat Med. 10: 1269) and has been observed in both primary and metastatic cancer cells, where its high expression is associated with poor prognosis in several cancer types (e.g., Ino et al. (2008) Clin Cancer Res. 14(8): 2310; Brandacher et al. (2006) Clin. Cancer Res. 12(4): 1144). TDO2, while less well studied, is also expressed in a wide variety of human tumors and is associated with tumoral immune resistance in mice. IDO1 is induced in tumors by proinflammatory cytokines, including type I and type II interferons that are produced by infiltrating lymphocytes (Tnani and Bayard (1999) Biochim Biophys Acta 1451(1): 59; Mellor and Munn (2004) Nat Rev Immunol 4(10): 762; Munn (2012) Front Biosci. 4: 734) and TGF-beta (Pallotta et al. (2011) Nat Immunol. 12(9): 870). Certain oncogenic mutations can also lead to increased IDO1 expression, e.g., loss of the tumor suppressor BinI (Muller et al. (2005) Nat Med. 11(3): 312) or activating mutations in KIT (Balachandran et al. (2011) Nat Med. 17(9): 1094).

IDO1 or TDO2 activity in the tumor suppresses anti-tumor T cell responses through local reduction in Trp levels and accumulation of Kyn in the TME and tumor draining lymph nodes (TDLN). Local Trp depletion causes a cellular stress response that includes activation of the stress response kinase general control nonderepressible 2 (GCN2), leading to T cell anergy in some tumors, reduced T cell proliferation, and enhanced Treg differentiation and activation (e.g., Ino et al. (2008) Clin Cancer Res. 14(8): 2310; Brandacher et al. (2006) Clin. Cancer Res. 12(4): 1144), and a recent report has shown that reduction of IDO1 expression in human gastrointestinal tumors goes along with an increased infiltration of tumors by effector T cells (Balachandran et al. (2011) Nat Med. 17(9): 1094). Kyn accumulation results in increased activation of the aryl hydrocarbon receptor (AHR), which promotes Treg differentiation and an immunosuppressive phenotype in dendritic cells (DCs) and macrophages. Thus, the combined effects of Trp depletion and Kyn accumulation in the TME dampen anti-tumor immune responses, allowing continued growth of the tumor. Trp catabolism's immunosuppressive role in cancer has made it a promising therapeutic target for anti-cancer therapy.

A significant amount of preclinical data has been published that further validates the role of IDO1 in the anti-tumor immune response. For example, forced IDO1 induction in cancer cells was shown to confer a survival advantage (Uyttenhove et al. (2003) Nat Med. 10: 1269). Other in vivo studies showed that IDO1 inhibitors cause lymphocyte dependent reduction in tumor growth by lowering kynurenine levels (Liu et al. (2010) Blood. 115(17): 3520). Studies also highlighted the scope for IDO1 inhibitors to work synergistically in combination (e.g., WO 2016/181348), for example, with agents that promote tumor antigenicity like irradiation, chemotherapy or vaccines (Koblish et al. (2010) Mol Cancer Ther. 9(2): 489, Hou et al. (2007) Cancer Res. 67(2): 792; Sharma et al. (2009) Blood 113(24): 6102).

Many diseases are associated with abnormal cellular responses, proliferation and evasion of programmed cell-death, triggered by mediated events as described above and herein. Cancer is an abnormal growth of cells which tend to proliferate in an uncontrolled way and, in some cases, to metastasize (spread). Cancer is not one disease. It is a group of more than 100 different and distinctive diseases. Cancer can involve any tissue of the body and have many different forms in each body area. Most cancers are named for the type of cell or organ in which they start. If a cancer spreads (metastasizes), the new tumor bears the same name as the original (primary) tumor. The frequency of a particular cancer may depend on the gender.

Therapies targeting PD-L1 and IDO1 separately have shown anti-tumor effects in pre-clinical studies and in the clinic, but improving their anti-tumor efficacy and the proportion of responders remain important goals. Accordingly, there remains a need to develop novel therapeutic options for the treatment of cancers. Furthermore, there is a need for therapies having greater efficacy than existing therapies. Preferred combination therapies of the present invention show greater efficacy than treatment with either therapeutic agent alone.

### SUMMARY OF THE INVENTION

Each of the embodiments described below can be combined with any other embodiment described herein not inconsistent with the embodiment with which it is combined. Furthermore, each of the embodiments described herein envisions within its scope pharmaceutically acceptable salts of the compounds described herein. Accordingly, the phrase “or a pharmaceutically acceptable salt thereof” is implicit in the description of all compounds described herein. Embodiments within an aspect as described below can be combined with any other embodiments not inconsistent within the same aspect or a different aspect.

The present invention arises out of the discovery that a subject having a cancer can be treated with a combination comprising an anti-PD-1 axis binding antagonist and an IDO1 inhibitor. Thus, in a first aspect, the present invention provides a method comprising administering to the subject an anti-PD-L1 antibody, or an antigen-binding fragment thereof, and an IDO1 inhibitor for treating a cancer in a subject in need thereof. Particularly provided is a method for treating an IDO1-positive cancer that induces an escape pathway to checkpoint inhibitor treatment in a subject in need thereof, comprising administering to the subject an anti-PD-L1 antibody, or an antigen-binding fragment thereof, and an IDO1 inhibitor, wherein the IDO1 inhibitor 3-(5-fluoro-1H-indol-3-yl)pyrrolididine-2,5-dione is excluded. The anti-PD-L1 antibody and the IDO1 inhibitor are administered in amounts that together are effective in treating cancer. Also provided are methods of inhibiting tumor growth or progression in a subject who has malignant cells. Also provided are methods of inhibiting metastasis of malignant cells in a subject. Also provided are methods of inducing tumor regression in a subject who has malignant cells. The combination treatment results in an objective response, preferably a complete response or partial response in the subject. In another aspect of all the embodiments of this invention, and in combination with any other aspects not inconsistent, the method provides an objective response rate of the patients under the treatment of at least about 20%, at least about 30%, at least about 40% or at least about 50%. In another aspect of all the embodiments of this invention, and in combination with any other aspects not inconsistent, the method provides a median overall survival time of the patients under the treatment of at least about 1 month, at least about 2 months, at least about 3 months, at least about 4 months, at least about 5 months, at least about 6 months, at least about 7 months, at least about 8 months, at least about 9 months, at least about 10 months or at least about 11 months. The objective response rate and/or median overall survival time are improved in comparison to either therapy with an anti-PD-1 axis binding antagonist or an IDO1 inhibitor.

In some embodiments, the cancer is identified as PD-L1-positive cancerous disease. In some embodiments, the cancer is identified as IDO1-positive cancerous disease. In some embodiments, the cancer is an IDO1-positive cancer that induces an escape pathway to checkpoint inhibitor treatment. In some embodiments, the subject suffers from an IDO1-expressing cancer whose IDO1 level exceeds an IDO1 level predetermined prior to administering to the subject the anti-PD-L1 antibody and/or the IDO1 inhibitor. In other words, the IDO1-positive cancer shows an IDO1 expression that exceeds an IDO1 level predetermined prior to administering to the subject the anti-PD-L1 antibody and/or the IDO1 inhibitor.

The cancer can be a metastatic or locally advanced unresectable solid tumor. Specific types of cancer to be treated according to the invention include, but are not limited to, a cancer selected from malignant melanoma, acute myelogenous leukemia, pancreatic, colorectal, lung, prostate, cervical, brain, liver, head and neck, endometrial, esophageal, breast, and ovarian cancers, and histological subtypes thereof.

The anti-PD-L1 antibody and IDO1 inhibitor can be administered in a first-line, second-line or higher treatment of the cancer. In some embodiments, the cancer is resistant to prior cancer therapy. The combination therapy of the invention can also be used in the treatment of a subject with the cancer who has been previously treated with one or more chemo- or immunotherapies, or underwent radiotherapy but failed with any such previous treatment.

In some embodiments, the subject to be treated is human. In some embodiments, the anti-PD-L1 antibody is used in the treatment of a human subject. In some embodiments, PD-L1 is human PD-L1.

In some embodiments, the anti-PD-L1 antibody mediates antibody-dependent cell-mediated cytotoxicity (ADCC). Nevertheless, such ADCC-mediating anti-PD-L1 antibody is not toxic or does not show increased toxicity. In some embodiments, the anti-PD-L1 antibody shows cross-reactivity in mice and rhesus monkeys. In some embodiments, the anti-PD-L1 antibody comprises a heavy chain, which comprises three complementarity determining regions (CDRs) having amino acid sequences of SEQ ID NOs: 1, 2 and 3, and a light chain, which comprises three complementarity determining regions (CDRs) having amino acid sequences of SEQ ID NOs: 4, 5 and 6. The anti-PD-L1 antibody preferably comprises the heavy chain having amino acid sequences of SEQ ID NOs: 7 or 8 and the light chain having amino acid sequence of SEQ ID NO: 9. In some preferred embodiments, the anti-PD-L1 antibody is avelumab.

In some embodiment, the anti-PD-L1 antibody is administered intravenously (e.g., as an intravenous infusion) or subcutaneously. Preferably, the anti-PD-L1 antibody is administered as an intravenous infusion. More preferably, the inhibitor is administered for 50-80 minutes. Most preferably, the anti-PD-L1 antibody is administered via intravenous infusion over 50-80 minutes, highly preferably as a one-hour intravenous infusion. In some embodiment, the anti-PD-L1 antibody is administered at a dose of about 10 mg/kg body weight every other week (i.e., every two weeks, or “Q2W”). In some embodiments, the anti-PD-L1 antibody is administered at a dose of about 800 mg Q2W.

In some aspects, the IDO1 inhibitor is a dual IDO1/TDO2 inhibitor. In some aspects, the IDO1 inhibitor 3-(5-fluoro-1H-indol-3-yl)pyrrolididine-2,5-dione is excluded. In some aspects, the IDO1 inhibitor is 4-fluoro-4-[2-[5H-imidazo[4,3-a]isoindol-5-yl]ethyl]cyclohexane-1-sulfonamide (“Compound 1”) or a pharmaceutically acceptable salt thereof. In some aspects, the IDO1 inhibitor is c-4-fluoro-t-4-[(S)-2-(5H-imidazo[5,1-a]isoindol-5-yl)-ethyl]-cyclohexane-r-1-sulfonic acid amide (“Compound 2”) or a pharmaceutically acceptable salt thereof. In some embodiments, the IDO1 inhibitor is administered orally. In some embodiments, the IDO1 inhibitor is administered at a dose of about 0.01 to about 200 mg/kg twice daily (i.e., “BID”), e.g., for 3 or 4 weeks. Preferably, the IDO1 inhibitor is administered at a dose of about 0.01 mg/kg BID, 0.1 mg/kg BID, 1 mg/kg BID, 10 mg/kg BID, 100 mg/kg BID, 150 mg/kg BID or 200 mg/kg BID. In some embodiments, the IDO1 inhibitor is administered at a dose of about 10 to about 1000 mg twice daily (i.e., “BID”), e.g., for 3 or 4 weeks. Preferably, the IDO1 inhibitor is administered at a dose of about 100 mg BID, 200 mg BID, 300 mg BID, 400 mg BID, 500 mg BID, 600 mg BID, 700 mg, 800 mg BID or 900 mg/kg BID.

In a preferred embodiment, the dose for the IDO1 inhibitor is about 0.01 to 200 mg/kg orally BID or about 100 to 900 mg orally BID, and the dose for avelumab is 10 mg/kg IV Q2W or about 800 mg Q2W. In another preferred embodiment, the recommended phase 2 dose for the IDO1 inhibitor is about 0.01 to 200 mg/kg orally BID or about 100 to 900 mg orally BID, and the recommended phase 2 dose for avelumab is 10 mg/kg IV Q2W or about 800 mg Q2W.

In other embodiments, the anti-PD-L1 antibody and IDO1 inhibitor are used in combination with chemotherapy (CT), radiotherapy (RT) or chemoradiotherapy (CRT). The chemotherapeutic agent can be etoposide, topotecan, irinotecan, fluorouracil, a platin, an anthracycline, and a combination thereof. The radiotherapy can be a treatment given with electrons, photons, protons, alfa-emitters, other ions, radio-nucleotides, boron capture neutrons and combinations thereof. In some embodiments, the radiotherapy comprises about 35-70 Gy/20-35 fractions.

In a further aspect, the anti-PD-L1 antibody and the IDO1 inhibitor are administered sequentially in either order or substantially simultaneously. In some embodiments, the combination regimen comprises the steps of: (a) under the direction or control of a physician, the subject receiving the PD-L1 antibody prior to first receipt of the IDO1 inhibitor; and (b) under the direction or control of a physician, the subject receiving the IDO1 inhibitor. In some embodiments, the combination regimen comprises the steps of: (a) under the direction or control of a physician, the subject receiving the IDO1 inhibitor prior to first receipt of the PD-L1 antibody; and (b) under the direction or control of a physician, the subject receiving the PD-L1 antibody. In some embodiments, the combination regimen comprises the steps of: (a) prescribing the subject to self-administer, and verifying that the subject has self-administered, the PD-L1 antibody prior to first administration of the IDO1 inhibitor; and (b) administering the IDO1 inhibitor to the subject. In some embodiments, the combination regimen comprises the steps of: (a) prescribing the subject to self-administer, and verifying that the subject has self-administered, the IDO1 inhibitor prior to first administration of the PD-L1 antibody; and (b) administering the PD-L1 antibody to the subject. In some embodiments, the combination regimen comprises the steps of: (a) after the subject has received prior cancer therapy and/or the PD-L1 antibody prior to the first administration of the IDO1 inhibitor, optionally determining that an IDO1 level in a cancer sample isolated from the subject exceeds an IDO1 level predetermined prior to the first receipt of the prior cancer therapy and/or the anti-PD-L1 antibody, and (b) administering the IDO1 inhibitor to the subject. In some embodiments, the combination regimen comprises, after the subject has received the IDO1 inhibitor prior to first administration of the anti-PD-L1 antibody, administering the anti-PD-L1 antibody to the subject. It shall be understood that combination regimen and method can be interchangeably used in this context.

In a further aspect, the invention also relates to a method for advertising an anti-PD-L1 antibody in combination with an IDO1 inhibitor, comprising promoting, to a target audience, the use of the combination for treating a subject with a cancer based on PD-L1 or IDO1 expression in samples taken from the subject. Similarly provided is a method for advertising an IDO1 inhibitor in combination with an anti-PD-L1 antibody, comprising promoting, to a target audience, the use of the combination for treating a subject with a cancer based on PD-L1 or IDO1 expression in samples taken from the subject. Also provided is method for advertising a combination comprising an anti-PD-L1 antibody and an IDO1 inhibitor, comprising promoting, to a target audience, the use of the combination for treating a subject with a cancer based on PD-L1 or IDO1 expression in samples taken from the subject. The PD-L1 or IDO1 expression can be determined by immunohistochemistry, e.g., using one or more primary anti-PD-L1 antibodies or anti-IDO1 antibodies, FACS or LC/MS/MS.

In a further aspect, the invention relates to a method for predicting the likelihood that a subject suffering from a cancer, which is a candidate for treatment with an anti-PD-L1 antibody and an IDO1 inhibitor, will respond to the treatment, comprising determining the IDO1 expression by means of surrogate markers, which are tryptophan and kynurenine or the concentration ratio thereof, using LC/MS/MS, in a sample obtained from the subject, wherein a higher expression, as compared to a predetermined value, indicates that the subject is likely to respond to the treatment. The method is particularly suited to predict the likelihood that a subject suffering from an IDO1-positive cancer, which induces an escape pathway to checkpoint inhibitor treatment and is a candidate for treatment with an anti-PD-L1 antibody and an IDO1 inhibitor, wherein the IDO1 inhibitor 3-(5-fluoro-1H-indol-3-yl)pyrrolididine-2,5-dione is excluded, will respond to the treatment.

In a further aspect, the invention relates to a method for monitoring the likelihood of response to a treatment of a cancer, which is mediated and/or propagated by IDO1 expression, wherein a kynurenine plasma level is determined in a sample withdrawn from a subject in need of such treatment with an anti-PD-L1 antibody and an IDO1 inhibitor, wherein a decrease in the kynurenine plasma level relative to a predetermined level indicates an increased likelihood that the subject responds to the treatment with the anti-PD-L1 antibody and the IDO1 inhibitor.

The method is particularly suited to monitor the likelihood of response to a treatment of a cancer with an anti-PD-L1 antibody and an IDO1 inhibitor, wherein such cancer is mediated and/or propagated by IDO1 expression and induces an escape pathway to checkpoint inhibitor treatment, and wherein the IDO1 inhibitor 3-(5-fluoro-1H-indol-3-yl)pyrrolididine-2,5-dione is excluded. In some embodiments, the decrease is at least 10%, 20%, 30%, 40% or 50%.

In a further aspect, the invention relates to the use of IDO1 as biomarker for a therapeutic anti-PD-L1 antibody.

Provided herein is also a pharmaceutical composition comprising an anti-PD-L1 antibody, a IDO1 inhibitor and at least a pharmaceutically acceptable excipient or adjuvant. The anti-PD-L1 antibody and the IDO1 inhibitor can be provided in a single or separate unit dosage forms. Also provided herein is a composition comprising an anti-PD-L1 antibody for use in the treatment of an IDO1-positive cancer that induces an escape pathway to checkpoint inhibitor treatment, wherein the composition is administered in combination with an IDO1 inhibitor, and wherein the IDO1 inhibitor 3-(5-fluoro-1H-indol-3-yl)pyrrolididine-2,5-dione is excluded. Also provided herein is a composition comprising an IDO1 inhibitor for use in the treatment of an IDO1-positive cancer that induces an escape pathway to checkpoint inhibitor treatment, wherein the composition is administered in combination with an anti-PD-L1 antibody, and wherein the IDO1 inhibitor 3-(5-fluoro-1H-indol-3-yl)pyrrolididine-2,5-dione is excluded.

Also provided herein is an anti-PD-L1 antibody in combination with an IDO1 inhibitor for use as a medicament, particularly for use in the treatment of cancer. Similarly, an IDO1 inhibitor is provided in combination with an anti-PD-L1 antibody for use as a medicament, particularly for use in the treatment of cancer. Also provided is a combination comprising an anti-PD-L1 antibody and an IDO1 inhibitor, for any purpose, for use as a medicament (i.e., a combination for use as a medicament, comprising an anti-PD-L1 antibody and an IDO1 inhibitor), or in the treatment of cancer. The combination of the anti-PD-L1 antibody and the IDO1 inhibitor can be provided in a single or separate unit dosage forms. Also provided is the use of a combination for the manufacture of a medicament for the treatment of cancer, comprising an anti-PD-L1 antibody and an IDO1 inhibitor. Also provided herein is a combination for use in the treatment of an IDO1-positive cancer that induces an escape pathway to checkpoint inhibitor treatment, comprising an anti-PD-L1 antibody and an IDO1 inhibitor, wherein the IDO1 inhibitor 3-(5-fluoro-1H-indol-3-yl)pyrrolididine-2,5-dione is excluded.

In a further aspect, the invention relates to a kit comprising an anti-PD-L1 antibody and a package insert comprising instructions for using the anti-PD-L1 antibody in combination with an IDO1 inhibitor to treat or delay progression of a cancer in a subject. Also provided is a kit comprising an IDO1 inhibitor and a package insert comprising instructions for using the IDO1 inhibitor in combination with an anti-PD-L1 antibody to treat or delay progression of a cancer in a subject. Also provided is a kit comprising an anti-PD-L1 antibody and an IDO1 inhibitor, and a package insert comprising instructions for using the anti-PD-L1 antibody and the IDO1 inhibitor to treat or delay progression of a cancer in a subject. The kit can comprise a first container, a second container and a package insert, wherein the first container comprises at least one dose of a medicament comprising an anti-PD-L1 antibody, the second container comprises at least one dose of a medicament comprising an IDO1 inhibitor, and the package insert comprises instructions for treating the subject for cancer using the medicaments. The instructions can state that the medicaments are intended for use in treating a subject having a cancer that tests positive for PD-L1 or IDO1 expression, e.g., by an immunohistochemical (IHC) assay, FACS or LC/MS/MS. In the various embodiments above, the kit is suited to treat and delay progression of an IDO1-positive cancer that induces an escape pathway to checkpoint inhibitor treatment, wherein the IDO1 inhibitor 3-(5-fluoro-1H-indol-3-yl)pyrrolididine-2,5-dione is excluded. Accordingly, the instructions can state that the medicaments are intended for use in treating a subject having a cancer that tests positive for PD-L1 expression, e.g., by an immunohistochemical (IHC) assay.

In various embodiments, the anti-PD-L1 antibody administered to the subject is avelumab and/or the IDO1 inhibitor is c-4-fluoro-t-4-[(S)-2-(5H-imidazo[5,1-a]isoindol-5-yl)-ethyl]-cyclohexane-r-1-sulfonic acid amide, or a pharmaceutically acceptable salt thereof.

### BRIEF DESCRIPTION OF THE FIGURES

FIG. 1 shows the heavy chain sequence of avelumab. (A) SEQ ID NO: 7 represents the full length heavy chain sequence of avelumab. The CDRs having the amino acid sequences of SEQ ID NOs: 1, 2 and 3 are marked by underlining. (B) SEQ ID NO: 8 represents the heavy chain sequence of avelumab without the C-terminal lysine. The CDRs having the amino acid sequences of SEQ ID NOs: 1, 2 and 3 are marked by underlining.

FIG. 2 (SEQ ID NO: 9) shows the light chain sequence of avelumab. The CDRs having the amino acid sequences of SEQ ID NOs: 4, 5 and 6 are marked by underlining.

FIG. 3 shows that the combination therapy with M4112 and avelumab inhibited tumor growth and extended survival in the CT26-KSA tumor model. CT26-KSA colon carcinoma cells (1×10⁶ cells in 0.1 mL PBS) were inoculated s.c. in the right flank of 8-12-week-old BALB/c female mice. On Day 0, when average tumor volume was approximately 150-200 mm³, mice were randomized into treatment groups (N=10 mice/group) and therapy was initiated. Mice received the following treatments: 1) M4112 vehicle (0.1 mL; p.o.; 2×/day for 3 weeks)+isotype control (400 μg; i.v.; Days 0, 3, 6); 2) M4112 vehicle (0.1 mL; p.o.; 2×/day for 3 weeks)+avelumab (400 μg; i.v.; Days 0, 3, 6); 3) M4112 (200 mg/kg; p.o.; 2×/day for 3 weeks)+isotype control (400 μg; i.v.; Days 0, 3, 6); or 4) M4112 (200 mg/kg; p.o.; 2×/day for 3 weeks)+avelumab (400 μg; i.v.; Days 0, 3, 6). Tumor volumes were measured twice per week throughout the study period. A) Average tumor volume over time. Error bars represent SEM. B) Individual tumor volumes over time. Each line represents data from a single mouse. P-values were calculated by 2-way RM ANOVA with Tukey's post-test. C) Kaplan-Meier survival curves. Median survival times are shown. n.s., not significant.

FIG. 4 shows the body weight of CT26-KSA tumor-bearing mice treated with M4112 and avelumab. CT26-KSA colon carcinoma cells (1×10⁶ cells in 0.1 mL PBS) were inoculated s.c. in the right flank of 8-12-week-old BALB/c female mice. On Day 0, when average tumor volume was approximately 150-200 mm³, mice were randomized into treatment groups (N=10 mice/group) and therapy was initiated. Mice received the following treatments: 1) M4112 vehicle (0.1 mL; p.o.; 2×/day for 3 weeks)+isotype control (400 μg; i.v.; Days 0, 3, 6); 2) M4112 vehicle (0.1 mL; p.o.; 2×/day for 3 weeks)+avelumab (400 μg; i.v.; Days 0, 3, 6); 3) M4112 (200 mg/kg; p.o.; 2×/day for 3 weeks)+isotype control (400 μg; i.v.; Days 0, 3, 6); or 4) M4112 (200 mg/kg; p.o.; 2×/day for 3 weeks)+avelumab (400 μg; i.v.; Days 0, 3, 6). Shown is the average percent change in body weight over time for each treatment group. Error bars represent SEM.

FIG. 5 shows that the combination therapy with M4112 and avelumab inhibits tumor growth in MC38 tumor-bearing mice. MC38 colon carcinoma cells (1×10⁶ cells in 0.1 mL PBS) were inoculated s.c. in the right flank of 8-12-week-old C57BL/6 female mice. On Day 0, when average tumor volume was approximately 50-100 mm³, mice were randomized into treatment groups (N=10 mice/group) and therapy was initiated. Mice received the following treatments: 1) M4112 vehicle (0.1 mL; p.o.; 2×/day for 4 weeks)+isotype control (400 μg; i.v.; Days 0, 2, 4); 2) M4112 vehicle (0.1 mL; p.o.; 2×/day for 4 weeks)+avelumab (400 μg; i.v.; Days 0, 2, 4); 3) M4112 (200 mg/kg; p.o.; 2×/day for 4 weeks)+isotype control (400 μg; i.v.; Days 0, 2, 4); or 4) M4112 (200 mg/kg; p.o.; 2×/day for 4 weeks)+avelumab (400 μg; i.v.; Days 0, 2, 4). Tumor volumes were measured twice per week throughout the study period. A) Average tumor volume over time. Error bars represent SEM. B) Individual tumor volumes over time. Each line represents data from a single mouse. P-values were calculated by 2-way RM ANOVA with Tukey's post-test. C) Kaplan-Meier survival curves. Median survival times are shown.

FIG. 6 shows the body weight of MC38 tumor-bearing mice treated with M4112 and avelumab. MC38 cells (1×10⁶ cells in 0.1 mL PBS) were inoculated s.c. in the right flank of 8-12-week-old C57BL/6 female mice. On Day 0, when average tumor volume was app. 50-100 mm³, mice were randomized into treatment groups (N=10 mice/group) and therapy was initiated. Mice received the following treatments: 1) M4112 vehicle (0.1 mL; p.o.; 2×/day for 4 weeks)+isotype control (400 μg; i.v.; Days 0, 2, 4); 2) M4112 vehicle (0.1 mL; p.o.; 2×/day for 4 weeks)+avelumab (400 μg; i.v.; Days 0, 2, 4); 3) M4112 (200 mg/kg; p.o.; 2×/day for 4 weeks)+isotype control (400 μg; i.v.; Days 0, 2, 4); or 4) M4112 (200 mg/kg; p.o.; 2×/day for 4 weeks)+avelumab (400 μg; i.v.; Days 0, 2, 4). Shown is average percent change in body weight over time for each treatment group. Error bars represent SEM.

FIG. 7 shows the correlation between IDO1 level and M4112 activity in combination with avelumab in one-way MLR assays. High levels of IDO1 in mDCs correlate with suppressed avelumab activity (IFNγ production) in the one-way MLR assay, which is reversed in an avelumab dose-dependent manner when M4112 is used in combination. Prior to the one-way MLR assay, imDCs from donor 17988 were treated with 1 μg/mL LPS for two days to obtain mDCs. Cells were stained with either an isotype control Phycoerythrin (PE) antibody or an anti-IDO1-PE antibody. (A-B) Stained cells were analyzed via FACS. Purple (solid curve) represents isotype control stained cells and green (transparent curve) represents IDO1-PE stained cells. (C-D) Monocyte-depleted PBMCs from donor KP33289 were co-cultured with the IDO1-high mDCs (Experiment A) or the IDO1-low mDCs (Experiment B) at a 2.3:1 ratio in MLR assays. IDO1 inhibitor M4112 was tested at 5 μM; Vehicle (Veh): DMSO. Avelumab was tested at 8 different doses (ng/mL), as indicated. On (C) Day 4 (Experiment A) or (D) Day 3 (Experiment B) of the MLR assay, IFNγ levels (μg/mL) were measured in the co-culture supernatant via IFNγ ELISA. Symbols and error bars indicate mean and SEM. A non-linear fit line was applied to the graph.

### DETAILED DESCRIPTION OF THE INVENTION

The present invention may be understood more readily by reference to the following detailed description of the preferred embodiments of the invention and the Examples included herein. It is to be understood that the terminology used herein is for the purpose of describing specific embodiments only and is not intended to be limiting. It is further to be understood that unless specifically defined herein, the terminology used herein is to be given its traditional meaning as known in the relevant art. So that the invention may be more readily understood, certain technical and scientific terms are specifically defined below. Unless specifically defined elsewhere in this document, all other technical and scientific terms used herein have the meaning commonly understood by one of ordinary skill in the art to which this invention belongs.

### Definitions

The following definitions are provided to assist the reader. Unless otherwise defined, all terms of art, notations, and other scientific or medical terms or terminology used herein are intended to have the meanings commonly understood by those of skill in the chemical and medical arts. In some cases, terms with commonly understood meanings are defined herein for clarity and/or for ready reference, and the inclusion of such definitions herein should not be construed as representing a substantial difference over the definition of the term as generally understood in the art.

“A”, “an”, and “the” include plural referents unless the context clearly dictates otherwise. Thus, for example, reference to an antibody refers to one or more antibodies or at least one antibody. As such, the terms “a” (or “an”), “one or more”, and “at least one” are used interchangeably herein.

“About” when used to modify a numerically defined parameter (e.g., the dose of an anti-PD-L1 antibody or IDO1 inhibitor, or the length of treatment time with a combination therapy described herein) means that the parameter may vary by as much as 10% below or above the stated numerical value for that parameter. For example, a dose of about 10 mg/kg may vary between 9 mg/kg and 11 mg/kg.

“Administering” or “administration of” a drug to a patient (and grammatical equivalents of this phrase) refers to direct administration, which may be administration to a patient by a medical professional or may be self-administration, and/or indirect administration, which may be the act of prescribing a drug. E.g., a physician who instructs a patient to self-administer a drug or provides a patient with a prescription for a drug is administering the drug to the patient.

“Antibody” is an immunoglobulin molecule capable of specific binding to a target, such as a carbohydrate, polynucleotide, lipid, polypeptide, etc., through at least one antigen recognition site, located in the variable region of the immunoglobulin molecule. As used herein, the term “antibody” encompasses not only intact polyclonal or monoclonal antibodies, but also, unless otherwise specified, any antigen-binding fragment or antibody fragment thereof that competes with the intact antibody for specific binding, fusion proteins comprising an antigen-binding portion (e.g., antibody-drug conjugates), any other modified configuration of the immunoglobulin molecule that comprises an antigen recognition site, antibody compositions with poly-epitopic specificity, and multi-specific antibodies (e.g., bispecific antibodies).

“Antibody-dependent cell-mediated cytotoxicity” or “ADCC” refers to a form of cytotoxicity in which secreted Ig bound onto Fc receptors (FcRs) present on certain cytotoxic cells (e.g., natural killer (NK) cells, neutrophils, and macrophages) enable these cytotoxic effector cells to bind specifically to an antigen-bearing target cell and subsequently kill the target cell with cytotoxins. The antibodies arm the cytotoxic cells and are required for killing of the target cell by this mechanism. The primary cells for mediating ADCC, the NK cells, express FcγRIII only, whereas monocytes express FcγRI, FcγRII and FcγRIII. Fc expression on hematopoietic cells is summarized in Table 3 on page 464 of Ravetch and Kinet, Annu. Rev. Immunol. 9: 457-92 (1991).

“Antigen-binding fragment” of an antibody or “antibody fragment” comprises a portion of an intact antibody, which is still capable of antigen binding and/or the variable region of the intact antibody. Antigen-binding fragments include, for example, Fab, Fab′, F(ab′)₂, Fd, and Fv fragments, domain antibodies (dAbs, e.g., shark and camelid antibodies), fragments including complementarity determining regions (CDRs), single chain variable fragment antibodies (scFv), single-chain antibody molecules, multi-specific antibodies formed from antibody fragments, maxibodies, minibodies, intrabodies, diabodies, triabodies, tetrabodies, v-NAR and bis-scFv, linear antibodies (see e.g., U.S. Pat. No. 5,641,870, Example 2; Zapata et al. (1995) Protein Eng. 8HO: 1057), and polypeptides that contain at least a portion of an immunoglobulin that is sufficient to confer specific antigen binding to the polypeptide. Papain digestion of antibodies produces two identical antigen-binding fragments, called “Fab” fragments, and a residual “Fc” fragment, a designation reflecting the ability to crystallize readily. The Fab fragment consists of an entire L chain along with the variable region domain of the H chain (V_{H}), and the first constant domain of one heavy chain (C_{H}1). Each Fab fragment is monovalent with respect to antigen binding, i.e., it has a single antigen-binding site. Pepsin treatment of an antibody yields a single large F(ab′)₂ fragment, which roughly corresponds to two disulfide linked Fab fragments having different antigen-binding activity and is still capable of cross-linking antigen. Fab′ fragments differ from Fab fragments by having a few additional residues at the carboxy terminus of the C_{H}1 domain including one or more cysteines from the antibody hinge region. Fab′-SH is the designation herein for Fab′ in which the cysteine residue(s) of the constant domains bear a free thiol group. F(ab′)₂ antibody fragments were originally produced as pairs of Fab′ fragments which have hinge cysteines between them. Other chemical couplings of antibody fragments are also known.

“Anti-PD-L1 antibody” means an antibody that blocks binding of PD-L1 expressed on a cancer cell to PD-1. In any of the treatment method, medicaments and uses of the present invention in which a human subject is being treated, the anti-PD-L1 antibody specifically binds to human PD-L1 and blocks binding of human PD-L1 to human PD-1. The antibody may be a monoclonal antibody, human antibody, humanized antibody or chimeric antibody, and may include a human constant region. In some embodiments the human constant region is selected from the group consisting of IgG1, IgG2, IgG3 and IgG4 constant regions, and in preferred embodiments, the human constant region is an IgG1 or IgG4 constant region. In some embodiments, the antigen-binding fragment is selected from the group consisting of Fab, Fab′-SH, F(ab′)2, scFv and Fv fragments. Examples of monoclonal antibodies that bind to human PD-L1, and useful in the treatment method, medicaments and uses of the present invention, are described in WO 2007/005874, WO 2010/036959, WO 2010/077634, WO 2010/089411, WO 2013/019906, WO 2013/079174, WO 2014/100079, WO 2015/061668, and U.S. Pat. Nos. 8,552,154, 8,779,108 and 8,383,796. Specific anti-human PD-L1 monoclonal antibodies useful as the PD-L1 antibody in the treatment method, medicaments and uses of the present invention include, for example without limitation, avelumab (MSB0010718C), nivolumab (BMS-936558), MPDL3280A (an IgG1-engineered, anti-PD-L1 antibody), BMS-936559 (a fully human, anti-PD-L1, IgG4 monoclonal antibody), MED4736 (an engineered IgG1 kappa monoclonal antibody with triple mutations in the Fc domain to remove antibody-dependent, cell-mediated cytotoxic activity), and an antibody which comprises the heavy chain and light chain variable regions of SEQ ID NO:24 and SEQ ID NO:21, respectively, of WO 2013/019906.

“Biomarker” generally refers to biological molecules, and quantitative and qualitative measurements of the same, that are indicative of a disease state. “Prognostic biomarkers” correlate with disease outcome, independent of therapy. For example, tumor hypoxia is a negative prognostic marker—the higher the tumor hypoxia, the higher the likelihood that the outcome of the disease will be negative. “Predictive biomarkers” indicate whether a patient is likely to respond positively to a particular therapy. E.g., HER2 profiling is commonly used in breast cancer patients to determine if those patients are likely to respond to Herceptin (trastuzumab, Genentech). “Response biomarkers” provide a measure of the response to a therapy and so provide an indication of whether a therapy is working. For example, decreasing levels of prostate-specific antigen generally indicate that anti-cancer therapy for a prostate cancer patient is working. When a marker is used as a basis for identifying or selecting a patient for a treatment described herein, the marker can be measured before and/or during treatment, and the values obtained are used by a clinician in assessing any of the following: (a) probable or likely suitability of an individual to initially receive treatment(s); (b) probable or likely unsuitability of an individual to initially receive treatment(s); (c) responsiveness to treatment; (d) probable or likely suitability of an individual to continue to receive treatment(s); (e) probable or likely unsuitability of an individual to continue to receive treatment(s); (f) adjusting dosage; (g) predicting likelihood of clinical benefits; or (h) toxicity. As would be well understood by one in the art, measurement of a biomarker in a clinical setting is a clear indication that this parameter was used as a basis for initiating, continuing, adjusting and/or ceasing administration of the treatments described herein.

“Blood” refers to all components of blood circulating in a subject including, but not limited to, red blood cells, white blood cells, plasma, clotting factors, small proteins, platelets and/or cryoprecipitate. This is typically the type of blood which is donated when a human patient gives blood. Plasma is known in the art as the yellow liquid component of blood, in which the blood cells in whole blood are typically suspended. It makes up about 55% of the total blood volume. Blood plasma can be prepared by spinning a tube of fresh blood containing an anti-coagulant in a centrifuge until the blood cells fall to the bottom of the tube. The blood plasma is then poured or drawn off. Blood plasma has a density of approximately 1025 kg/m³ or 1.025 kg/l.

“Cancer”, “cancerous”, or “malignant” refer to or describe the physiological condition in mammals that is typically characterized by unregulated cell growth. Examples of cancer include but are not limited to, carcinoma, lymphoma, leukemia, blastoma, and sarcoma. More particular examples of such cancers include squamous cell carcinoma, myeloma, small-cell lung cancer, non-small cell lung cancer, glioma, hodgkin's lymphoma, non-hodgkin's lymphoma, acute myeloid leukemia, multiple myeloma, gastrointestinal (tract) cancer, renal cancer, ovarian cancer, liver cancer, lymphoblastic leukemia, lymphocytic leukemia, colorectal cancer, endometrial cancer, kidney cancer, prostate cancer, thyroid cancer, melanoma, chondrosarcoma, neuroblastoma, pancreatic cancer, glioblastoma multiforme, cervical cancer, brain cancer, stomach cancer, bladder cancer, hepatoma, breast cancer, colon carcinoma, and head and neck cancer.

“Checkpoint inhibitors” refer to a type of immunotherapy that help the immune system respond more strongly to a tumor. These drugs do not target the tumor directly but interfere with the ability of cancer cells to avoid immune system attack, thereby releasing brakes that keep T cells (a type of white blood cell and part of the immune system) from killing cancer cells. Non-limiting examples of checkpoint inhibitors are immune checkpoint inhibitors, such as antibodies against CTLA-4, PD-1 (e.g., nivolumab) or PD-L1 (e.g., atezolizumab, durvalumab, and avelumab).

“Chemotherapy” is a therapy involving a chemotherapeutic agent, which is a chemical compound useful in the treatment of cancer. Examples of chemotherapeutic agents include alkylating agents such as thiotepa and cyclophosphamide; alkyl sulfonates such as busulfan, improsulfan, and piposulfan; aziridines such as benzodopa, carboquone, meturedopa, and uredopa; ethylenimines and methylamelamines including altretamine, triethylenemelamine, trietylenephosphoramide, triethiylenethiophosphoramide, and trimethylolomelamine; acetogenins (especially bullatacin and bullatacinone); delta-9-tetrahydrocannabinol (dronabinol); beta-lapachone; lapachol; colchicines; betulinic acid; a camptothecin (including the synthetic analogue topotecan (CPT-11 (irinotecan), acetylcamptothecin, scopolectin, and 9-aminocamptothecin); bryostatin; pemetrexed; callystatin; CC-1065 (including its adozelesin, carzelesin, and bizelesin synthetic analogues); podophyllotoxin; podophyllinic acid; teniposide; cryptophycins (particularly, cryptophycin 1 and cryptophycin 8); dolastatin; duocarmycin (including the synthetic analogues KW-2189 and CB1-TM1); eleutherobin; pancratistatin; TLK-286; CDP323, an oral alpha-4 integrin inhibitor; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, and uracil mustard; nitrosureas such as carmustine, chlorozotocin, fotemustine, lomustine, nimustine, and ranimnustine; antibiotics such as the enediyne antibiotics (e.g., calicheamicin, especially calicheamicin gammall and calicheamicin omegall (see, e.g., Nicolaou et al. (1994) Angew. Chem Intl. Ed. Engl. 33: 183); dynemicin including dynemicin A; an esperamicin; as well as neocarzinostatin chromophore and related chromoprotein enediyne antibiotic chromophores, aclacinomysins, actinomycin, authramycin, azaserine, bleomycins, cactinomycin, carabicin, carminomycin, carzinophilin, chromomycinis, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin, doxorubicin HCl liposome injection, and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, and zorubicin; anti-metabolites such as methotrexate, gemcitabine, tegafur, capecitabine, an epothilone, and 5-fluorouracil (5-FU); folic acid analogues such as denopterin, methotrexate, pteropterin, and trimetrexate; purine analogs such as fludarabine, 6-mercaptopurine, thiamiprine, and thioguanine; pyrimidine analogs such as ancitabine, azacitidine, 6-azauridine, carmofur, cytarabine, dideoxyuridine, doxifluridine, enocitabine, floxuridine, and imatinib (a 2-phenylaminopyrimidine derivative), as well as other c-Kit inhibitors; anti-adrenals such as aminoglutethimide, mitotane, and trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; eflornithine; elliptinium acetate; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; 2-ethylhydrazide; procarbazine; PSK polysaccharide complex (JHS Natural Products, Eugene, Oreg.); razoxane; rhizoxin; sizofiran; spirogermanium; tenuazonic acid; triaziquone; 2,2′,2″-trichlorotriethylamine; trichothecenes (especially, T-2 toxin, verracurin A, roridin A, and anguidine); urethan; vindesine; dacarbazine; mannomustine; mitobronitol; mitolactol; pipobroman; gacytosine; arabinoside (“Ara-C”); thiotepa; taxoids, e.g., paclitaxel, albumin-engineered nanoparticle formulation of paclitaxel, and doxetaxel; chloranbucil; 6-thioguanine; mercaptopurine; methotrexate; platinum analogs such as cisplatin and carboplatin; vinblastine; platinum; etoposide (VP-16); ifosfamide; mitoxantrone; vincristine; oxaliplatin; leucovovin; vinorelbine; novantrone; edatrexate; daunomycin; aminopterin; ibandronate; topoisomerase inhibitor RFS 2000; difluoromethylornithine (DMFO); retinoids such as retinoic acid; pharmaceutically acceptable salts, acids or derivatives of any of the above; as well as combinations of two or more of the above such as CHOP, an abbreviation for a combined therapy of cyclophosphamide, doxorubicin, vincristine and prednisolone, or FOLFOX, an abbreviation for a treatment regimen with oxaliplatin combined with 5-FU and leucovovin.

“Clinical outcome”, “clinical parameter”, “clinical response”, or “clinical endpoint” refers to any clinical observation or measurement relating to a patient's reaction to a therapy. Non-limiting examples of clinical outcomes include tumor response (TR), overall survival (OS), progression free survival (PFS), disease free survival, time to tumor recurrence (TTR), time to tumor progression (TTP), relative risk (RR), toxicity, or side effect.

“Combination” as used herein refers to the provision of a first active modality in addition to another active modality. Contemplated with the scope of the combinations described herein, are any regimen of combination modalities or partners (i.e., active compounds, components or agents), such as a combination of an IDO1 inhibitor and a PD-1 axis binding antagonist, encompassed in single or multiple compositions. It is understood that any modalities within a single composition, formulation or unit dosage form (i.e., a fixed-dose combination) must have the identical dose regimen and route of delivery. it is not intended to imply that the modalities must be formulated for delivery together (e.g., in the same composition, formulation or unit dosage form). The combined modalities can be manufactured and/or formulated by the same or different manufacturers. The combination partners may thus be, e.g., entirely separate pharmaceutical dosage forms or pharmaceutical compositions that are also sold independently of each other.

“Combination therapy”, “in combination with” or “in conjunction with” as used herein denotes any form of concurrent, parallel, simultaneous, sequential or intermittent treatment with at least two distinct treatment modalities (i.e., compounds, components, targeted agents or therapeutic agents). As such, the terms refer to administration of one treatment modality before, during, or after administration of the other treatment modality to the subject. The modalities in combination can be administered in any order. The therapeutically active modalities are administered together (e.g., simultaneously in the same or separate compositions, formulations or unit dosage forms) or separately (e.g., on the same day or on different days and in any order as according to an appropriate dosing protocol for the separate compositions, formulations or unit dosage forms) in a manner and dosing regimen prescribed by a medical care taker or according to a regulatory agency. In general, each treatment modality will be administered at a dose and/or on a time schedule determined for that treatment modality. Optionally, three or more modalities may be used in a combination therapy. Additionally, the combination therapies provided herein may be used in conjunction with other types of treatment. For example, other anti-cancer treatment may be selected from the group consisting of chemotherapy, surgery, radiotherapy (radiation) and/or hormone therapy, amongst other treatments associated with the current standard of care for the subject.

“Complete response” or “complete remission” refers to the disappearance of all signs of cancer in response to treatment. This does not always mean the cancer has been cured.

“Comprising”, as used herein, is intended to mean that the compositions and methods include the recited elements, but not excluding others. “Consisting essentially of”, when used to define compositions and methods, shall mean excluding other elements of any essential significance to the composition or method. “Consisting of” shall mean excluding more than trace elements of other ingredients for claimed compositions and substantial method steps. Embodiments defined by each of these transition terms are within the scope of this invention. Accordingly, it is intended that the methods and compositions can include additional steps and components (comprising) or alternatively including steps and compositions of no significance (consisting essentially of) or alternatively, intending only the stated method steps or compositions (consisting of).

“Dose” and “dosage” refer to a specific amount of active or therapeutic agents for administration. Such amounts are included in a “dosage form,” which refers to physically discrete units suitable as unitary dosages for human subjects and other mammals, each unit containing a predetermined quantity of active agent calculated to produce the desired onset, tolerability, and therapeutic effects, in association with one or more suitable pharmaceutical excipients such as carriers.

“Enhancing T-cell function” means to induce, cause or stimulate a T-cell to have a sustained or amplified biological function, or renew or reactivate exhausted or inactive T-cells. Examples of enhancing T-cell function include: increased secretion of y-interferon from CD8+ T-cells, increased proliferation, increased antigen responsiveness (e.g., viral, pathogen, or tumor clearance) relative to such levels before the intervention. In one embodiment, the level of. enhancement is as least 50%, alternatively 60%, 70%, 80%, 90%, 100%, 1 20%, 150%, 200%. The manner of measuring this enhancement is known to one of ordinary skill in the art.

“Fc” is a fragment comprising the carboxy-terminal portions of both H chains held together by disulfides. The effector functions of antibodies are determined by sequences in the Fc region, the region which is also recognized by Fc receptors (FcR) found on certain cell types.

“Functional fragments” of the antibodies of the invention comprise a portion of an intact antibody, generally including the antigen-binding or variable region of the intact antibody or the Fc region of an antibody which retains or has modified FcR binding capability. Examples of functional antibody fragments include linear antibodies, single-chain antibody molecules, and multi-specific antibodies formed from antibody fragments.

“Fv” is the minimum antibody fragment, which contains a complete antigen-recognition and antigen-binding site. This fragment consists of a dimer of one heavy- and one light-chain variable region domain in tight, non-covalent association. From the folding of these two domains emanate six hypervariable loops (3 loops each from the H and L chain) that contribute the amino acid residues for antigen binding and confer antigen-binding specificity to the antibody. However, even a single variable domain (or half of an Fv comprising only three HVRs specific for an antigen) has the ability to recognize and bind antigen, although at a lower affinity than the entire binding site.

“Human antibody” is an antibody that possesses an amino-acid sequence corresponding to that of an antibody produced by a human and/or has been made using any of the techniques for making human antibodies as disclosed herein. This definition of a human antibody specifically excludes a humanized antibody comprising non-human antigen-binding residues. Human antibodies can be produced using various techniques known in the art, including phage-display libraries (see e.g., Hoogenboom and Winter (1991), JMB 227: 381; Marks et al. (1991) JMB 222: 581). Also available for the preparation of human monoclonal antibodies are methods described in Cole et al. (1985) Monoclonal Antibodies and Cancer Therapy, Alan R. Liss, page 77; Boerner et al. (1991), J. Immunol 147(l): 86; van Dijk and van de Winkel (2001) Curr. Opin. Pharmacol 5: 368). Human antibodies can be prepared by administering the antigen to a transgenic animal that has been modified to produce such antibodies in response to antigenic challenge but whose endogenous loci have been disabled, e.g., immunized xenomice (see e.g., U.S. Pat. Nos. 6,075,181; and 6,150,584 regarding XENOMOUSE technology). See also, for example, L1 et al. (2006) PNAS USA, 103: 3557, regarding human antibodies generated via a human B-cell hybridoma technology.

“Humanized” forms of non-human (e.g., murine) antibodies are chimeric antibodies that contain minimal sequence derived from non-human immunoglobulin. In one embodiment, a humanized antibody is a human immunoglobulin (recipient antibody) in which residues from an HVR of the recipient are replaced by residues from an HVR of a non-human species (donor antibody) such as mouse, rat, rabbit, or non-human primate having the desired specificity, affinity and/or capacity. In some instances, framework (“FR”) residues of the human immunoglobulin are replaced by corresponding non-human residues. Furthermore, humanized antibodies may comprise residues that are not found in the recipient antibody or in the donor antibody. These modifications may be made to further refine antibody performance, such as binding affinity. In general, a humanized antibody will comprise substantially all of at least one, and typically two, variable domains, in which all or substantially all of the hypervariable loops correspond to those of a non-human immunoglobulin sequence, and all or substantially all of the FR regions are those of a human immunoglobulin sequence, although the FR regions may include one or more individual FR residue substitutions that improve antibody performance, such as binding affinity, isomerization, immunogenicity, etc. The number of these amino acid substitutions in the FR are typically no more than 6 in the H chain, and no more than 3 in the L chain. The humanized antibody optionally will also comprise at least a portion of an immunoglobulin constant region (Fc), typically that of a human immunoglobulin. For further details, see e.g., Jones et al. (1986) Nature 321: 522; Riechmann et al. (1988), Nature 332: 323; Presta (1992) Curr. Op. Struct. Biol. 2: 593; Vaswani and Hamilton (1998), Ann. Allergy, Asthma & Immunol. 1: 105; Harris (1995) Biochem. Soc. Transactions 23: 1035; Hurle and Gross (1994) Curr. Op. Biotech. 5: 428; and U.S. Pat. Nos. 6,982,321 and 7,087,409.

“IDO1 expression” as used herein means any detectable level of expression of IDO1 protein or IDO1 mRNA within a cell or tissue. IDO1 protein expression may be detected with a diagnostic IDO1 antibody in an IHC assay of a tumor tissue section or by flow cytometry. Alternatively, IDO1 protein expression by tumor cells may be detected by PET imaging, using a binding agent (e.g., antibody fragment, affibody and the like) that specifically binds to IDO1. Techniques for detecting and measuring IDO1 mRNA expression include RT-PCR and real-time quantitative RT-PCR.

“IDO1 inhibitor” refers to a compound that has a biological effect to inhibit or significantly reduce or down-regulate the expression of the gene encoding for IDO1 and/or the expression of IDO1 and/or the biological activity of IDO1.

“IDO1-positive” cancer, including a “IDO1-positive” cancerous disease, is one comprising cells, which have IDO1 present in their cells. The term “IDO1-positive” also refers to a cancer that produces sufficient levels of IDO1 in the cells thereof, such that an IDO1 inhibitor has a therapeutic effect, mediated by the binding of the said IDO1 inhibitor to IDO1.

“Immunoglobulin” (Ig) is used interchangeably with “antibody” herein. The basic 4-chain antibody unit is a heterotetrameric glycoprotein composed of two identical light (L) chains and two identical heavy (H) chains. An IgM antibody consists of 5 of the basic heterotetramer units along with an additional polypeptide called a J chain, and contains 10 antigen binding sites, while IgA antibodies comprise from 2-5 of the basic 4-chain units which can polymerize to form polyvalent assemblages in combination with the J chain. In the case of IgGs, the 4-chain unit is generally about 150,000 Daltons. Each L chain is linked to an H chain by one covalent disulfide bond, while the two H chains are linked to each other by one or more disulfide bonds depending on the H chain isotype. Each H and L chain also has regularly spaced intra-chain disulfide bridges. Each H chain has, at the N-terminus, a variable domain (V_{H}) followed by three constant domains (C_{H}) for each of the α and γ chains and four C_{H} domains for μ and ε isotypes. Each L chain has at the N-terminus, a variable domain (V_{L}) followed by a constant domain at its other end. The V_{L} is aligned with the V_{H} and the C_{L} is aligned with the first constant domain of the heavy chain (C_{H}1). Particular amino acid residues are believed to form an interface between the light chain and heavy chain variable domains. The pairing of a V_{H} and V_{L} together forms a single antigen-binding site. For the structure and properties of the different classes of antibodies, see e.g., Basic and Clinical Immunology, 8^{th} Edition, Sties et al. (eds.), Appleton & Lange, Norwalk, Conn., 1994, page 71 and Chapter 6. The L chain from any vertebrate species can be assigned to one of two clearly distinct types, called kappa and lambda, based on the amino acid sequences of their constant domains. Depending on the amino acid sequence of the constant domain of their heavy chains (C_{H}), immunoglobulins can be assigned to different classes or isotypes. There are five classes of immunoglobulins: IgA, IgD, IgE, IgG and IgM, having heavy chains designated α, δ, ε, γ and μ, respectively. The γ and α classes are further divided into subclasses on the basis of relatively minor differences in the C_{H} sequence and function, e.g., humans express the following subclasses: IgG1, IgG2A, IgG2B, IgG3, IgG4, IgA1, and IgK1.

“Immunotherapy” refers to the treatment of a subject by a method comprising inducing, enhancing, suppressing, or otherwise modifying an immune response, including checkpoint inhibitor treatment.

“Infusion” or “infusing” refers to the introduction of a drug-containing solution into the body through a vein for therapeutic purposes. Generally, this is achieved via an intravenous bag.

“Isolated” refers to molecules or biological or cellular materials being substantially free from other materials. In one aspect, the term “isolated” refers to nucleic acid, such as DNA or RNA, or protein or polypeptide, or cell or cellular organelle, or tissue or organ, separated from other DNAs or RNAs, or proteins or polypeptides, or cells or cellular organelles, or tissues or organs, respectively, that are present in the natural source. The term “isolated” also refers to a nucleic acid or peptide that is substantially free of cellular material, viral material, or culture medium when produced by recombinant DNA techniques, or chemical precursors or other chemicals when chemically synthesized. Moreover, an “isolated nucleic acid” is meant to include nucleic acid fragments which are not naturally occurring as fragments and would not be found in the natural state. The term “isolated” is also used herein to refer to polypeptides which are isolated from other cellular proteins and is meant to encompass both purified and recombinant polypeptides. The term “isolated” is also used herein to refer to cells or tissues that are isolated from other cells or tissues and is meant to encompass both cultured and engineered cells or tissues. For example, an “isolated antibody” is one that has been identified, separated and/or recovered from a component of its production environment (e.g., natural or recombinant). Preferably, the isolated polypeptide is free of association with all other components from its production environment. Contaminant components of its production environment, such as that resulting from recombinant transfected cells, are materials that would typically interfere with research, diagnostic or therapeutic uses for the antibody, and may include enzymes, hormones, and other proteinaceous or non-proteinaceous solutes. In preferred embodiments, the polypeptide will be purified: (1) to greater than 95% by weight of antibody as determined by, for example, the Lowry method, and in some embodiments, to greater than 99% by weight; (1) to a degree sufficient to obtain at least 15 residues of N-terminal or internal amino acid sequence by use of a spinning cup sequenator, or (3) to homogeneity by SDS-PAGE under non-reducing or reducing conditions using Coomassie blue or, preferably, silver stain. The “isolated antibody” includes the antibody in-situ within recombinant cells since at least one component of the antibody's natural environment will not be present. Ordinarily, however, an isolated polypeptide or antibody will be prepared by at least one purification step.

“Metastatic” cancer refers to cancer which has spread from one part of the body (e.g., the lung) to another part of the body.

“Monoclonal antibody”, as used herein, refers to an antibody obtained from a population of substantially homogeneous antibodies, i.e., the individual antibodies comprising the population are identical except for possible naturally occurring mutations and/or post-translation modifications (e.g., isomerizations and amidations) that may be present in minor amounts. Monoclonal antibodies are highly specific, being directed against a single antigenic site. In contrast to polyclonal antibody preparations, which typically include different antibodies directed against different determinants (epitopes), each monoclonal antibody is directed against a single determinant on the antigen. In addition to their specificity, the monoclonal antibodies are advantageous in that they are synthesized by the hybridoma culture and uncontaminated by other immunoglobulins. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by a variety of techniques, including, for example, the hybridoma method (e.g., Kohler and Milstein (1975) Nature 256: 495; Hongo et al. (1995) Hybridoma 14 (3): 253; Harlow et al. (1988) Antibodies: A Laboratory Manual (Cold Spring Harbor Laboratory Press, 2^{nd} ed.; Hammerling et al. (1981) In: Monoclonal Antibodies and T-Cell Hybridomas 563 (Elsevier, N.Y.), recombinant DNA methods (see e.g., U.S. Pat. No. 4,816,567), phage-display technologies (see e.g., Clackson et al. (1991) Nature 352: 624; Marks et al. (1992) JMB 222: 581; Sidhu et al. (2004) JMB 338(2): 299; Lee et al. (2004) JMB 340(5): 1073; Fellouse (2004) PNAS USA 101(34): 12467; and Lee et al. (2004) J. Immunol. Methods 284(1-2): 119), and technologies for producing human or human-like antibodies in animals that have parts or all of the human immunoglobulin loci or genes encoding human immunoglobulin sequences (see e.g., WO 1998/24893; WO 1996/34096; WO 1996/33735; WO 1991/10741; Jakobovits et al. (1993) PNAS USA 90: 2551; Jakobovits et al. (1993) Nature 362: 255; Bruggemann et al. (1993) Year in Immunol. 7: 33; U.S. Pat. Nos. 5,545,807; 5,545,806; 5,569,825; 5,625,126; 5,633,425; and U.S. Pat. No. 5,661,016; Marks et al. (1992) Bio/Technology 10: 779; Lonberg et al. (1994) Nature 368: 856; Morrison (1994) Nature 368: 812; Fishwild et al. (1996) Nature Biotechnol. 14: 845; Neuberger (1996), Nature Biotechnol. 14: 826; and Lonberg and Huszar (1995), Intern. Rev. Immunol. 13: 65-93). The monoclonal antibodies herein specifically include chimeric antibodies (immunoglobulins) in which a portion of the heavy and/or light chain is identical with or homologous to corresponding sequences in antibodies derived from a particular species or belonging to a particular antibody class or subclass, while the remainder of the chain(s) is (are) identical with or homologous to corresponding sequences in antibodies derived from another species or belonging to another antibody class or subclass, as well as fragments of such antibodies, so long as they exhibit the desired biological activity (see e.g., U.S. Pat. No. 4,816,567; Morrison et al. (1984) PNAS USA, 81: 6851).

“Objective response” refers to a measurable response, including complete response (CR) or partial response (PR).

“Overall Survival” (OS) refers to the time from patient enrollment to death or censored at the date last known alive. OS includes a prolongation in life expectancy as compared to naive or untreated individuals or patients. Overall survival refers to the situation wherein a patient remains alive for a defined period of time, such as one year, five years, etc., e.g., from the time of diagnosis or treatment.

“Partial response” refers to a decrease in the size of one or more tumors or lesions, or in the extent of cancer in the body, in response to treatment, as defined in the RECIST v1.1 guideline.

“Patient” and “subject” are used interchangeably herein to refer to a mammal in need of treatment for a cancer. Generally, the patient is a human diagnosed or at risk for suffering from one or more symptoms of a cancer. In certain embodiments a “patient” or “subject” may refer to a non-human mammal, such as a non-human primate species, a dog, cat, rabbit, pig, cow; rodents, including mouse, rat or hamster, or animals used in screening, characterizing, and evaluating drugs and therapies.

“PD-1 axis binding antagonist” as used herein refers to a molecule that inhibits the interaction of a PD-1 axis binding partner with one or more of its binding partners, so as to remove T-cell dysfunction resulting from signaling on the PD-1 signaling axis, with a result being to restore or enhance T-cell function. As used herein, a PD-1 axis binding antagonist includes a PD-1 binding antagonist, a PD-L1 binding antagonist and a PD-L2 binding antagonist. In one embodiment, the PD-1 axis binding antagonist is a PD-L1 binding antagonist. In one embodiment, the PD-L1 binding antagonist is avelumab.

“PD-L1 expression” as used herein means any detectable level of expression of PD-L1 protein on the cell surface or of PD-L1 mRNA within a cell or tissue. PD-L1 protein expression may be detected with a diagnostic PD-L1 antibody in an IHC assay of a tumor tissue section or by flow cytometry. Alternatively, PD-L1 protein expression by tumor cells may be detected by PET imaging, using a binding agent (e.g., antibody fragment, affibody and the like) that specifically binds to PD-L1. Techniques for detecting and measuring PD-L1 mRNA expression include RT-PCR and real-time quantitative RT-PCR.

“PD-L1-positive” cancer, including a “PD-L1-positive” cancerous disease, is one comprising cells, which have PD-L1 present at their cell surface. The term “PD-L1-positive” also refers to a cancer that produces sufficient levels of PD-L1 at the surface of cells thereof, such that an anti-PD-L1 antibody has a therapeutic effect, mediated by the binding of the said anti-PD-L1 antibody to PD-L1.

“Pharmaceutically acceptable” indicates that the substance or composition must be compatible chemically and/or toxicologically, with the other ingredients comprised in a formulation, and/or the mammal being treated therewith. In other words, the substance or composition must be chemically and/or toxicologically suitable for the treatment of mammals.

“Pharmaceutically acceptable adjuvant” refers to any and all substances which enhance the body's immune response to an antigen. Non-limiting examples of pharmaceutically acceptable adjuvants are: Alum, Freund's Incomplete Adjuvant, MF59, synthetic analogs of dsRNA such as poly(I:C), bacterial LPS, bacterial flagellin, imidazolquinolines, oligodeoxynucleotides containing specific CpG motifs, fragments of bacterial cell walls such as muramyl dipeptide and Quil-A©.

“Pharmaceutically acceptable carrier” or “pharmaceutically acceptable diluent” means any and all solvents, dispersion media, coatings, antibacterial and antifungal agents, isotonic and absorption delaying agents, compatible with pharmaceutical administration. Examples of pharmaceutically acceptable carriers include one or more of water, saline, phosphate buffered saline, dextrose, glycerol, ethanol and the like, as well as combinations thereof. The use of such media and agents for pharmaceutically active substances is well known in the art. Acceptable carriers, excipients, or stabilizers are nontoxic to recipients at the dosages and concentrations employed and, without limiting the scope of the present invention, include: additional buffering agents; preservatives; co-solvents; antioxidants, including ascorbic acid and methionine; chelating agents such as EDTA; metal complexes (e.g., Zn-protein complexes); biodegradable polymers, such as polyesters; salt-forming counterions, such as sodium, polyhydric sugar alcohols; amino acids, such as alanine, glycine, glutamine, asparagine, histidine, arginine, lysine, ornithine, leucine, 2-phenylalanine, glutamic acid, and threonine; organic sugars or sugar alcohols, such as lactitol, stachyose, mannose, sorbose, xylose, ribose, ribitol, myoinisitose, myoinisitol, galactose, galactitol, glycerol, cyclitols (e.g., inositol), polyethylene glycol; sulfur containing reducing agents, such as urea, glutathione, thioctic acid, sodium thioglycolate, thioglycerol, [alpha]-monothioglycerol, and sodium thio sulfate; low molecular weight proteins, such as human serum albumin, bovine serum albumin, gelatin, or other immunoglobulins; and hydrophilic polymers, such as polyvinylpyrrolidone. Other pharmaceutically acceptable carriers, excipients, or stabilizers, such as those described in Remington's Pharmaceutical Sciences 16th edition, Osol, A. Ed. (1980) may also be included in a pharmaceutical composition described herein, provided that they do not adversely affect the desired characteristics of the pharmaceutical composition.

“Predetermined value” or “predetermined level” for IDO1 as used herein, is so chosen that a patient with a level of IDO1 higher than the predetermined value is likely to experience a more desirable clinical outcome than patients with levels of IDO1 lower than the predetermined value, or vice-versa. Levels of proteins and/or RNA, such as those disclosed in the present invention, are associated with clinical outcomes. One of skill in the art can determine such predetermined values by measuring levels of IDO1 in a patient population to provide a predetermined value. Optionally, a predetermined value for IDO1 level in one patient population can be compared to that from another to optimize the predetermined value to provide higher predictive value. In various embodiments, a predetermined value refers to value(s) that best separate patients into a group with more desirable clinical outcomes and a group with less desirable clinical outcomes. Such predetermined value(s) can be mathematically or statistically determined with methods well known in the art in view of this disclosure.

“Progressive disease” or “disease that has progressed” refers to the appearance of one more new lesions or tumors and/or the unequivocal progression of existing non-target lesions as defined in the RECIST v1.1 guideline. Progressive disease or disease that has progressed can also refer to a tumor growth of more than 20 percent since treatment began, either due to an increase in mass or in spread of the tumor.

“Progression free survival” (PFS) refers to the time from enrollment to disease progression or death. PFS is generally measured using the Kaplan-Meier method and Response Evaluation Criteria in Solid Tumors (RECIST) 1.1 standards. Generally, progression free survival refers to the situation, wherein a patient remains alive, without the cancer getting worse.

“RECIST” means Response Evaluation Criteria in Solid Tumors. RECIST guideline, criteria, or standard, describes a standard approach to solid tumor measurement and definitions for objective assessment of change in tumor size for use in adult and pediatric cancer clinical trials. RECIST v1.1 means version 1.1 of the revised RECIST guideline and it is published in European Journal of Cancers 45 (2009) 228-247.

“Recurrent” cancer is one which has regrown, either at the initial site or at a distant site, after a response to initial therapy, such as surgery. A locally “recurrent” cancer is cancer that returns after treatment in the same place as a previously treated cancer.

“Reduction” of a symptom or symptoms (and grammatical equivalents of this phrase) refers to decreasing the severity or frequency of the symptom(s), or elimination of the symptom(s).

“Resistant” tumors or cancers are tumors or cancers, which cannot, or can no longer, be treated with anti-cancer agents, such as immunotherapeutic agents, checkpoint inhibitors or IDO1 inhibitors. Resistance may already be present before the first treatment attempt with an anti-cancer agent. Resistance may also be acquired after the initial treatment with an anti-cancer agent, in some instances as a result of the treatment.

“Respond favorably” generally refers to causing a beneficial state in a subject. With respect to cancer treatment, the term refers to providing a therapeutic effect on the subject. Positive therapeutic effects in cancer can be measured in a number of ways (See, Weber, 2009. J Nucl Med. 50 Suppl 1:1S-10S). For example, tumor growth inhibition, molecular marker expression, serum marker expression, and molecular imaging techniques can all be used to assess therapeutic efficacy of an anti-cancer therapeutic. With respect to tumor growth inhibition, according to NCI standards, a T/C s 42% is the minimum level of anti-tumor activity. A T/C<10% is considered a high anti-tumor activity level, with T/C (%)=Median tumor volume of the treated/Median tumor volume of the control×100. A favorable response can be assessed, for example, by increased progression-free survival (PFS), disease-free survival (DFS), or overall survival (OS), complete response (CR), partial response (PR), or, in some cases, stable disease (SD), a decrease in progressive disease (PD), a reduced time to progression (TTP) or any combination thereof.

“Serum” refers to the clear liquid that can be separated from clotted blood. Serum differs from plasma, the liquid portion of normal unclotted blood containing the red and white cells and platelets. Serum is the component that is neither a blood cell (serum does not contain white or red blood cells) nor a clotting factor. It is the blood plasma not including the fibrinogens that help in the formation of blood clots. It is the clot that makes the difference between serum and plasma.

“Single-chain Fv”, also abbreviated as “sFv” or “scFv”, are antibody fragments that comprise the V_{H} and V_{L} antibody domains connected into a single polypeptide chain. Preferably, the sFv polypeptide further comprises a polypeptide linker between the V_{H} and V_{L} domains which enables the sFv to form the desired structure for antigen binding. For a review of the sFv, see e.g., Pluckthun (1994), In: The Pharmacology of Monoclonal Antibodies, vol. 113, Rosenburg and Moore (eds.), Springer-Verlag, New York, pp. 269.

“Suitable for therapy” or “suitable for treatment” shall mean that the patient is likely to exhibit one or more desirable clinical outcomes as compared to patients having the same cancer and receiving the same therapy but possessing a different characteristic that is under consideration for the purpose of the comparison. In one aspect, the characteristic under consideration is a genetic polymorphism or a somatic mutation (see e.g., Samsami et al. (2009) J Reproductive Med 54(1): 25). In another aspect, the characteristic under consideration is the expression level of a gene or a polypeptide. In one aspect, a more desirable clinical outcome is relatively higher likelihood of or relatively better tumor response such as tumor load reduction. In another aspect, a more desirable clinical outcome is relatively longer overall survival. In yet another aspect, a more desirable clinical outcome is relatively longer progression free survival or time to tumor progression. In yet another aspect, a more desirable clinical outcome is relatively longer disease free survival. In another aspect, a more desirable clinical outcome is relative reduction or delay in tumor recurrence. In another aspect, a more desirable clinical outcome is relatively decreased metastasis. In another aspect, a more desirable clinical outcome is relatively lower relative risk. In yet another aspect, a more desirable clinical outcome is relatively reduced toxicity or side effects. In some embodiments, more than one clinical outcomes are considered simultaneously. In one such aspect, a patient possessing a characteristic, such as a genotype of a genetic polymorphism, may exhibit more than one more desirable clinical outcomes as compared to patients having the same cancer and receiving the same therapy but not possessing the characteristic. As defined herein, the patient is considered suitable for the therapy. In another such aspect, a patient possessing a characteristic may exhibit one or more desirable clinical outcomes but simultaneously exhibit one or more less desirable clinical outcomes. The clinical outcomes will then be considered collectively, and a decision as to whether the patient is suitable for the therapy will be made accordingly, taking into account the patient's specific situation and the relevance of the clinical outcomes. In some embodiments, progression free survival or overall survival is weighted more heavily than tumor response in a collective decision making.

“Sustained response” means a sustained therapeutic effect after cessation of treatment with a therapeutic agent, or a combination therapy described herein. In some embodiments, the sustained response has a duration that is at least the same as the treatment duration, or at least 1.5, 2.0, 2.5 or 3 times longer than the treatment duration.

“Systemic” treatment is a treatment, in which the drug substance travels through the bloodstream, reaching and affecting cells all over the body.

“Therapeutically effective amount” of an anti-PD-L1 antibody or antigen-binding fragment thereof, or an IDO1 inhibitor, in each case of the invention, refers to an amount effective, at dosages and for periods of time necessary, whereby, when administered to a patient with a cancer according to a method, combination or combination therapy, the method, combination or combination therapy, will have the intended therapeutic effect, e.g., alleviation, amelioration, palliation, or elimination of one or more manifestations of the cancer in the patient, or any other clinical result in the course of treating a cancer patient. A therapeutic effect does not necessarily occur by administration of one dose, and may occur only after administration of a series of doses. Thus, a therapeutically effective amount may be administered in one or more administrations. Such therapeutically effective amount may vary according to factors such as the disease state, age, sex, and weight of the individual, and the ability of an anti-PD-L1 antibody or antigen-binding fragment thereof, or an IDO1 inhibitor, to elicit a desired response in the individual. A therapeutically effective amount is also one in which any toxic or detrimental effects of an anti-PD-L1 antibody or antigen-binding fragment thereof, or an IDO1 inhibitor, are outweighed by the therapeutically beneficial effects.

“Treating” or “treatment of” a condition or patient refers to taking steps to obtain beneficial or desired results, including clinical results. For purposes of this invention, beneficial or desired clinical results include, but are not limited to, alleviation, amelioration of one or more symptoms of a cancer; diminishment of extent of disease; delay or slowing of disease progression; amelioration, palliation, or stabilization of the disease state; or other beneficial results. It is to be appreciated that references to “treating” or “treatment” include prophylaxis as well as the alleviation of established symptoms of a condition. “Treating” or “treatment” of a state, disorder or condition therefore includes: (1) preventing or delaying the appearance of clinical symptoms of the state, disorder or condition developing in a subject that may be afflicted with or predisposed to the state, disorder or condition but does not yet experience or display clinical or subclinical symptoms of the state, disorder or condition, (2) inhibiting the state, disorder or condition, i.e., arresting, reducing or delaying the development of the disease or a relapse thereof (in case of maintenance treatment) or at least one clinical or subclinical symptom thereof, or (3) relieving or attenuating the disease, i.e., causing regression of the state, disorder or condition or at least one of its clinical or subclinical symptoms.

“Tumor” as it applies to a subject diagnosed with, or suspected of having, a cancer refers to a malignant or potentially malignant neoplasm or tissue mass of any size, and includes primary tumors and secondary neoplasms. A solid tumor is an abnormal growth or mass of tissue that usually does not contain cysts or liquid areas. Different types of solid tumors are named for the type of cells that form them. Examples of solid tumors are sarcomas, carcinomas, and lymphomas. Leukemias (cancers of the blood) generally do not form solid tumors.

“Unit dosage form” as used herein refers to a physically discrete unit of therapeutic formulation appropriate for the subject to be treated. It will be understood, however, that the total daily usage of the compositions of the present invention will be decided by the attending physician within the scope of sound medical judgment. The specific effective dose level for any particular subject or organism will depend upon a variety of factors including the disorder being treated and the severity of the disorder; activity of specific active agent employed; specific composition employed; age, body weight, general health, sex and diet of the subject; time of administration, and rate of excretion of the specific active agent employed; duration of the treatment; drugs and/or additional therapies used in combination or coincidental with specific compound(s) employed, and like factors well known in the medical arts.

“Variable” refers to the fact that certain segments of the variable domains differ extensively in sequence among antibodies. The V domain mediates antigen binding and defines the specificity of a particular antibody for its particular antigen. However, the variability is not evenly distributed across the entire span of the variable domains. Instead, it is concentrated in three segments called hypervariable regions (HVRs) both in the light-chain and the heavy chain variable domains. The more highly conserved portions of variable domains are called the framework regions (FR). The variable domains of native heavy and light chains each comprise four FR regions, largely adopting a beta-sheet configuration, connected by three HVRs, which form loops connecting, and in some cases forming part of, the beta-sheet structure. The HVRs in each chain are held together in close proximity by the FR regions and, with the HVRs from the other chain, contribute to the formation of the antigen-binding site of antibodies (see Kabat et al. (1991) Sequences of Immunological Interest, 5^{th} edition, National Institute of Health, Bethesda, Md.). The constant domains are not involved directly in the binding of antibody to an antigen, but exhibit various effector functions, such as participation of the antibody in antibody-dependent cellular toxicity.

“Variable region” or “variable domain” of an antibody refers to the amino-terminal domains of the heavy or light chain of the antibody. The variable domains of the heavy chain and light chain may be referred to as “V_{H}” and “V_{L}”, respectively. These domains are generally the most variable parts of the antibody (relative to other antibodies of the same class) and contain the antigen binding sites.

As used herein, a plurality of items, structural elements, compositional elements, and/or materials may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary.

Concentrations, amounts, and other numerical data may be expressed or presented herein in a range format. It is to be understood that such a range format is used merely for convenience and brevity and thus should be interpreted flexibly to include not only the numerical values explicitly recited as the limits of the range, but also to include all the individual numerical values or sub-ranges encompassed within that range as if each numerical value and sub-range is explicitly recited. As an illustration, a numerical range of “about 1 to about 5” should be interpreted to include not only the explicitly recited values of about 1 to about 5, but also include individual values and sub-ranges within the indicated range. Thus, included in this numerical range are individual values such as 2, 3, and 4 and sub-ranges such as from 1-3, from 2-4, and from 3-5, etc., as well as 1, 2, 3, 4, and 5, individually. This same principle applies to ranges reciting only one numerical value as a minimum or a maximum. Furthermore, such an interpretation should apply regardless of the breadth of the range or the characteristics being described.

### ABBREVIATIONS

Some abbreviations used in the description include:
ADCC: Antibody-dependent cell-mediated cytotoxicity
AHR: Aryl hydrocarbon receptor
ANOVA: Analysis of variance
BID: Twice daily
CDR: Complementarity determining region
CR: Complete response
CRC: Colorectal cancer
CRT: Chemoradiotherapy
CT: Chemotherapy
DC: Dendritic cell
DMEM: Dulbecco's Modified Eagle Medium
DMSO: Dimethyl sulfoxide
DNA: Deoxyribonucleic acid
ELISA: Enzyme-linked immunosorbent assay
FACS: Fluorescence-activated cell sorting
FBS: Fetal bovine serum
GCN2: General control nonderepressible 2
HPB: Hydroxypropyl beta cyclodextrin
IDO1: Indoleamine 2,3-dioxygenase
IFNγ: Interferon-gamma
Ig: Immunoglobulin
IHC: Immunohistochemistry
imDC: Immature dendritic cell
i.v. or IV: Intravenous
LPS: Lipopolysaccharide
M4112: Investigational small molecule IDO1/TDO2 inhibitor (“Compound 2”)
MLR: Mixed lymphocyte reaction
mCRC: Metastatic colorectal cancer
mDC: Mature dendritic cell
NAD: Nicotinamide adenine dinucleotide
NEAA: Non-essential amino acids
NK: Natural killers
OS: Overall survival
PBMC: Peripheral blood mononuclear cell
PBS: Phosphate buffered saline
PD-1: Programmed death 1
PD-L1: Programmed death ligand 1
PES: Polyester sulfone
PFS: Progression free survival
p.o.: Per os (by mouth)
PR: Partial response
QD: Once daily
QID: Four times a day
Q2W: Every two weeks
Q3W: Every three weeks
RM: Repeated measures
RNA: Ribonucleic acid
RO: Reverse osmosis
RP2D: Recommended phase 2 dose
RR: Relative risk
RT: Radiotherapy
s.c.: Subcutaneous
SEM: Standard error of the mean
SoC: Standard of care
SR: Sustained response
T/C: Treatment/control (the average tumor volume after treatment/average tumor volume after control treatment on last day of study)
TDLN: Tumor draining lymph nodes
TDO2: Tryptophan 2,3-dioxygenase
TME: Tumor microenvironment
TR: Tumor response
Treg: Regulatory T cell
TTP: Time to tumor progression
TTR: Time to tumor recurrence

### Descriptive Embodiments

### Therapeutic Combination and Method of Use Thereof

Tumors can evade immune surveillance by exploiting signaling pathways associated with immunosuppression. The immune checkpoint protein PD-L1 is commonly upregulated in tumors and signals through its receptor PD-1 to limit anti-tumor T cells responses by promoting T cell anergy and exhaustion. Similarly, high levels of the tryptophan catabolism enzymes IDO1 and TDO2 in the tumor are associated with suppression of anti-tumor T cell responses and poor prognosis in cancer. Without being bound by any theory, the inventors assume that, given the immunosuppressive actions of the PD-1/PD-L1 and IDO1/TDO2 pathways in cancer, the dual blockade of these pathways enhances anti-tumor immune responses over blockade of either pathway alone. Inhibition of IDO1 and TDO2 by an IDO inhibitor of the invention enhances T cell activity in the TME and promotes the immune response to the tumor.

Importantly, dual inhibition of both IDO1 and TDO2 is complementary and non-redundant, as some tumors were found to express only one of the enzymes. More importantly, mechanisms of action of IDO1/TDO2 and PD-L1/PD-1-targeting agents according to the invention are found to be complementary on T cells in combination therapy, which enhances anti-tumor activity over either therapy alone. Potentiation may be additive, or it may be synergistic. The potentiating effect of the combination therapy is at least additive. The present inventors have surprisingly found that the combination of an anti-PD-L1 antibody with an IDO1 inhibitor, wherein the IDO1 inhibitor 3-(5-fluoro-1H-indol-3-yl)pyrrolididine-2,5-dione is excluded, results in an improved treatment of an IDO1-positive cancer that induces an escape pathway to checkpoint inhibitor treatment.

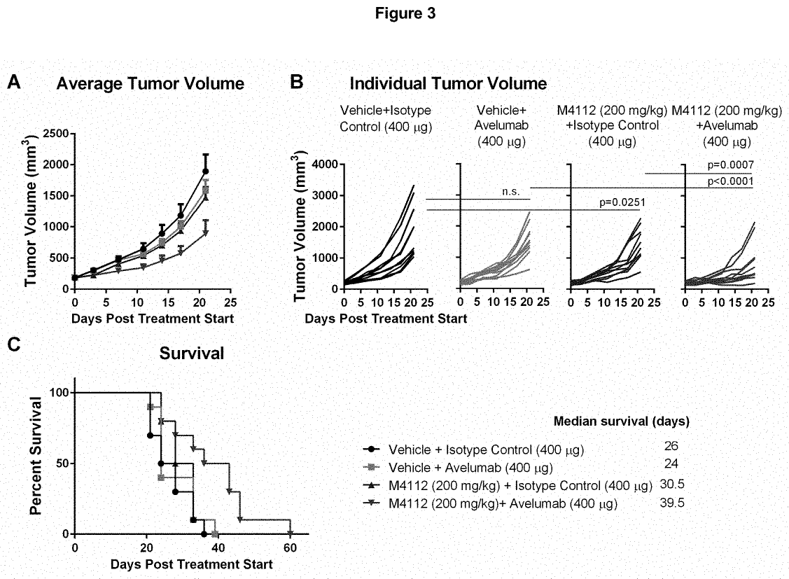

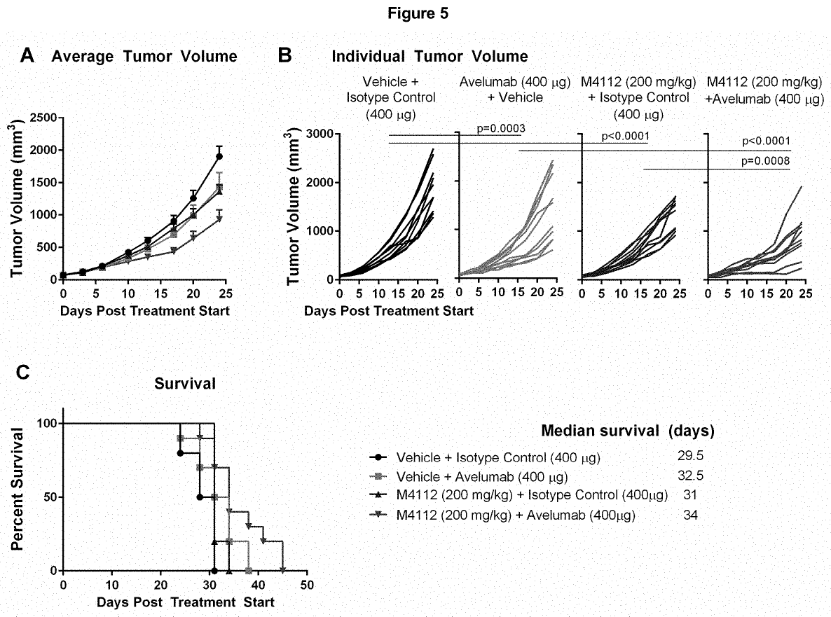

The present invention arose in part from the surprising discovery of a combination benefit for an IDO1 inhibitor and an PD-1 axis binding antagonist, preferably an anti-PD-L1 antibody, or an antigen-binding fragment thereof, each as defined herein, optionally in combination with radiotherapy, chemotherapy or chemoradiotherapy. Treatment schedule and doses were designed to reveal potential potentiating effect. Preclinical data demonstrated an additive until synergistic effect of the IDO1 inhibitor, particularly Compound 1 or 2, in combination with the PD-L1 antibody, particularly avelumab, versus the IDO1 inhibitor or avelumab (see e.g., FIG. 3 or 5). Further, initial results indicate that the combination therapy is effective at treating cancers such as colon cancer (see Example 2).

Thus, in one aspect, the present invention provides a method for treating a cancer in a subject in need thereof, comprising administering to the subject an anti-PD-L1 antibody, or an antigen-binding fragment or functional fragment thereof, and an IDO1 inhibitor. Particularly, the present invention provides a method for treating an IDO1-positive cancer that induces an escape pathway to checkpoint inhibitor treatment in a subject in need thereof, comprising administering to the subject an anti-PD-L1 antibody, or an antigen-binding fragment thereof, and an IDO1 inhibitor, wherein the IDO1 inhibitor 3-(5-fluoro-1H-indol-3-yl)pyrrolididine-2,5-dione is excluded. It shall be understood that a therapeutically effective amount of an anti-PD-L1 antibody and a therapeutically effective amount of an IDO1 inhibitor is to be applied in the method of the invention. In one embodiment, a therapeutically effective amount of the anti-PD-L1 antibody and a therapeutically effective amount of the IDO1 inhibitor are administered. The therapeutically effective amount is sufficient for treating one or more symptoms of a disease or disorder associated with PD-L1 and IDO1, respectively.

In one embodiment, the anti-PD-L1 antibody comprises a heavy chain, which comprises three complementarity determining regions having amino acid sequences of SEQ ID NOs: 1, 2 and 3, and alight chain, which comprises three complementarity determining regions having amino acid sequences of SEQ ID NOs: 4, 5 and 6.

In one embodiment, the anti-PD-L1 antibody is a monoclonal antibody. In one embodiment, the anti-PD-L1 antibody mediates antibody-dependent cell-mediated cytotoxicity (ADCC). In one embodiment, the anti-PD-L1 antibody is a human or humanized antibody. In one embodiment, the anti-PD-L1 antibody is an isolated antibody. In various embodiments, the anti-PD-L1 antibody is characterized by a combination of one or more of the foregoing features, as defined above.

In another aspect of this embodiment and in combination with any other aspects not inconsistent, the PD-1 axis binding antagonist is an anti PD-1 antibody selected from nivolumab and pembrolizumab. Nivolumab is disclosed in International Patent Publication No. WO 2006/121168, the disclosure of which is hereby incorporated by reference in its entirety. Pembrolizumab is disclosed in International Patent Publication No. WO 2009/114335, the disclosure of which is hereby incorporated by reference in its entirety.

In some embodiments, the PD-1 axis binding antagonist is an anti PD-L1 antibody selected from avelumab, durvalumab and atezolizumab. Avelumab is disclosed in International Patent Publication No. WO 2013/079174, the disclosure of which is hereby incorporated by reference in its entirety. Durvalumab is disclosed in International Patent Publication No. WO 2011/066389, the disclosure of which is hereby incorporated by reference in its entirety. Atezolizumab is disclosed in International Patent Publication No. WO 2010/077634, the disclosure of which is hereby incorporated by reference in its entirety.

Further exemplary PD-1 axis binding antagonists for use in the treatment method, medicaments and uses of the present invention are mAb7 (aka RN888), mAb15, AMP224 and YW243.55.S70. mAb7 (aka RN888) and mAb15 are disclosed in International Patent Publication No. WO 2016/092419, the disclosure of which is hereby incorporated by reference in its entirety. AMP224 is disclosed in International Patent Publication No. WO 2010/027827 and WO 2011/066342, the disclosure of which is hereby incorporated by reference in its entirety. YW243.55.S70 is disclosed in International Patent Publication No. WO 2010/077634, the disclosure of which is hereby incorporated by reference in its entirety.

Further antibodies or agents that target PD-1 or PD-L1 are, e.g., CT-011 (Curetech), BMS-936559 (Bristol-Myers Squibb), MGA-271 (Macrogenics), dacarbazine and Lambrolizumab (MK-3475).

In various embodiments, the anti-PD-L1 antibody is avelumab. Avelumab (formerly designated MSB0010718C) is a fully human monoclonal antibody of the immunoglobulin (Ig) G1 isotype (see e.g., WO 2013/079174). Avelumab selectively binds to PD-L1 and competitively blocks its interaction with PD-1. The mechanisms of action rely on the inhibition of PD-1/PD-L1 interaction and on natural killer (NK)-based ADCC (see e.g., Boyerinas et al. (2015) Cancer Immunol Res 3: 1148). Compared with anti-PD-1 antibodies that target T cells, avelumab targets tumor cells and therefore, it is expected to have fewer side effects, including a lower risk of autoimmune-related safety issues, as the blockade of PD-L1 leaves the PD-L2/PD-1 pathway intact to promote peripheral self-tolerance (see e.g., Latchman et al. (2001) Nat Immunol 2(3): 261).

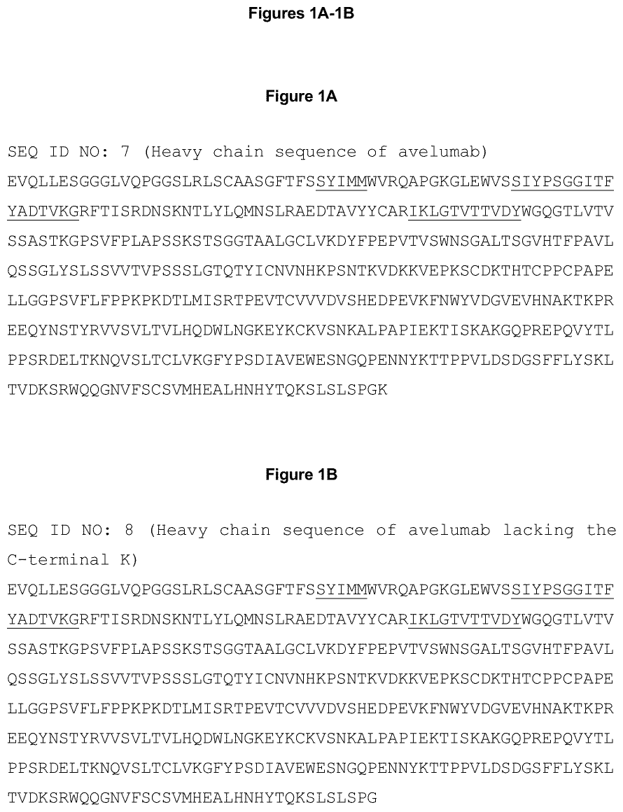

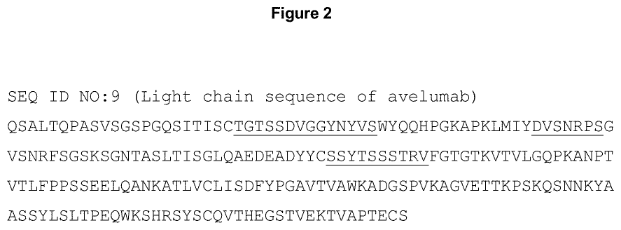

Avelumab, its sequence, and many of its properties have been described in WO 2013/079174, where it is designated A09-246-2 having the heavy and light chain sequences according to SEQ ID NOs: 32 and 33, as shown in FIG. 1 (SEQ ID NO: 7) and FIG. 2 (SEQ ID NO: 9), of this patent application. It is frequently observed, however, that in the course of antibody production the C-terminal lysine (K) of the heavy chain is cleaved off. This modification has no influence on the antibody-antigen binding. Therefore, in some embodiments the C-terminal lysine (K) of the heavy chain sequence of avelumab is absent. The heavy chain sequence of avelumab without the C-terminal lysine is shown in FIG. 1B (SEQ ID NO: 8), whereas FIG. 1A (SEQ ID NO: 7) shows the full length heavy chain sequence of avelumab. Further, as shown in WO 2013/079174, one of avelumab's properties is its ability to exert antibody-dependent cell-mediated cytotoxicity (ADCC), thereby directly acting on PD-L1 bearing tumor cells by inducing their lysis without showing any significant toxicity. In a preferred embodiment, the anti-PD-L1 antibody is avelumab, having the heavy and light chain sequences shown in FIG. 1A or 1B (SEQ ID NOs: 7 or 8), and FIG. 2 (SEQ ID NO: 9), or an antigen-binding fragment thereof.

In some aspects, the IDO1 inhibitor is a dual IDO1/TDO2 inhibitor.

In some embodiments, the IDO1 inhibitor 3-(5-fluoro-1H-indol-3-yl)pyrrolididine-2,5-dione is excluded.

In certain embodiments, the present invention provides antagonists of IDO. In some embodiments, such compounds include those of the formulae described herein, or a pharmaceutically acceptable salt thereof, wherein each variable is as defined and described herein.

Compounds of this invention include those described generally above, and are further illustrated by the classes, subclasses, and species disclosed herein. As used herein, the following definitions shall apply unless otherwise indicated. For purposes of this invention, the chemical elements are identified in accordance with the Periodic Table of the Elements, CAS version, Handbook of Chemistry and Physics, 75^{th} Ed. Additionally, general principles of organic chemistry are described in “Organic Chemistry”, Thomas Sorrell, University Science Books, Sausalito: 1999, and “March's Advanced Organic Chemistry”, 5^{th} Ed., Ed.: Smith, M. B. and March, J., John Wiley & Sons, New York: 2001, the entire contents of which are hereby incorporated by reference.

The term “aliphatic” or “aliphatic group”, as used herein, means a straight-chain (i.e., unbranched) or branched, substituted or unsubstituted hydrocarbon chain that is completely saturated or that contains one or more units of unsaturation, or a monocyclic hydrocarbon or bicyclic hydrocarbon that is completely saturated or that contains one or more units of unsaturation, but which is not aromatic (also referred to herein as “carbocycle” “cycloaliphatic” or “cycloalkyl”), that has a single point of attachment to the rest of the molecule. Unless otherwise specified, aliphatic groups contain 1-6 aliphatic carbon atoms. In some embodiments, aliphatic groups contain 1-5 aliphatic carbon atoms. In other embodiments, aliphatic groups contain 1-4 aliphatic carbon atoms. In still other embodiments, aliphatic groups contain 1-3 aliphatic carbon atoms, and in yet other embodiments, aliphatic groups contain 1-2 aliphatic carbon atoms. In some embodiments, “cycloaliphatic” (or “carbocycle” or “cycloalkyl”) refers to a monocyclic C₃-C₇ hydrocarbon that is completely saturated or that contains one or more units of unsaturation, but which is not aromatic, that has a single point of attachment to the rest of the molecule. Exemplary aliphatic groups are linear or branched, substituted or unsubstituted C₁-C₈ alkyl, C₂-C₈ alkenyl, C₂-C₈ alkynyl groups and hybrids thereof such as (cycloalkyl)alkyl, (cycloalkenyl)alkyl or (cycloalkyl)alkenyl.

The term “lower alkyl” refers to a C₁₋₄ straight or branched alkyl group. Exemplary lower alkyl groups are methyl, ethyl, propyl, isopropyl, butyl, isobutyl, and tert-butyl.

The term “lower haloalkyl” refers to a C₁₋₄ straight or branched alkyl group that is substituted with one or more halogen atoms.

The term “heteroatom” means one or more of oxygen, sulfur, nitrogen, or phosphorus (including, any oxidized form of nitrogen, sulfur, or phosphorus; the quaternized form of any basic nitrogen or; a substitutable nitrogen of a heterocyclic ring, for example N (as in 3,4-dihydro-2H-pyrrolyl), NH (as in pyrrolidinyl) or NR⁺ (as in N-substituted pyrrolidinyl)).

The term “unsaturated”, as used herein, means that a moiety has one or more units of unsaturation.

As used herein, the term “bivalent C₁₋₈ (or C₁₋₆) saturated or unsaturated, straight or branched, hydrocarbon chain”, refers to bivalent alkylene, alkenylene, and alkynylene chains that are straight or branched as defined herein.

The term “alkylene” refers to a bivalent alkyl group. An “alkylene chain” is a polymethylene group, i.e., —(CH₂)ₙ—, wherein n is a positive integer, preferably from 1 to 6, from 1 to 4, from 1 to 3, from 1 to 2, or from 2 to 3. A substituted alkylene chain is a polymethylene group in which one or more methylene hydrogen atoms are replaced with a substituent. Suitable substituents include those described below for a substituted aliphatic group.

The term “alkenylene” refers to a bivalent alkenyl group. A substituted alkenylene chain is a polymethylene group containing at least one double bond in which one or more hydrogen atoms are replaced with a substituent. Suitable substituents include those described below for a substituted aliphatic group. The term “alkynylene” refers to a bivalent alkynyl group. A substituted alkynylene chain is a group containing at least one triple bond in which one or more hydrogen atoms are replaced with a substituent. Suitable substituents include those described below for a substituted aliphatic group.

The term “halogen” means F, Cl, Br, or I.

The term “aryl” used alone or as part of a larger moiety as in “aralkyl”, “aralkoxy”, or “aryloxyalkyl”, refers to monocyclic and bicyclic ring systems having a total of five to fourteen ring members, wherein at least one ring in the system is aromatic and wherein each ring in the system contains three to seven ring members. The term “aryl” is used interchangeably with the term “aryl ring”. In certain embodiments of the present invention, “aryl” refers to an aromatic ring system. Exemplary aryl groups are phenyl, biphenyl, naphthyl, anthracyl and the like, which optionally includes one or more substituents. Also included within the scope of the term “aryl”, as it is used herein, is a group in which an aromatic ring is fused to one or more non-aromatic rings, such as indanyl, phthalimidyl, naphthimidyl, phenanthridinyl, or tetrahydronaphthyl, and the like.

The terms “heteroaryl” and “heteroar-”, used alone or as part of a larger moiety, e.g., “heteroaralkyl”, or “heteroaralkoxy”, refer to groups having 5 to 10 ring atoms, preferably 5, 6, or 9 ring atoms; having 6, 10, or 14 n electrons shared in a cyclic array; and having, in addition to carbon atoms, from one to five heteroatoms. The term “heteroatom” refers to nitrogen, oxygen, or sulfur, and includes any oxidized form of nitrogen or sulfur, and any quaternized form of a basic nitrogen. Heteroaryl groups include, without limitation, thienyl, furanyl, pyrrolyl, imidazolyl, pyrazolyl, triazolyl, tetrazolyl, oxazolyl, isoxazolyl, oxadiazolyl, thiazolyl, isothiazolyl, thiadiazolyl, pyridyl, pyridazinyl, pyrimidinyl, pyrazinyl, indolizinyl, purinyl, naphthyridinyl, and pteridinyl. The terms “heteroaryl” and “heteroaryl-”, as used herein, also include groups in which a heteroaromatic ring is fused to one or more aryl, cycloaliphatic, or heterocyclyl rings, where the radical or point of attachment is on the heteroaromatic ring. Non-limiting examples include indolyl, isoindolyl, benzothienyl, benzofuranyl, dibenzofuranyl, indazolyl, benzimidazolyl, benzthiazolyl, quinolyl, isoquinolyl, cinnolinyl, phthalazinyl, quinazolinyl, quinoxalinyl, 4H-quinolizinyl, carbazolyl, acridinyl, phenazinyl, phenothiazinyl, phenoxazinyl, tetrahydroquinolinyl, tetrahydroisoquinolinyl, and pyrido[2,3-b]-1,4-oxazin-3(4H)-one. A heteroaryl group is optionally mono- or bicyclic. The term “heteroaryl” is used interchangeably with the terms “heteroaryl ring”, “heteroaryl group”, or “heteroaromatic”, any of which terms include rings that are optionally substituted. The term “heteroaralkyl” refers to an alkyl group substituted by a heteroaryl, wherein the alkyl and heteroaryl portions independently are optionally substituted.

As used herein, the terms “heterocycle”, “heterocyclyl”, “heterocyclic radical”, and “heterocyclic ring” are used interchangeably and refer to a stable 5- to 7-membered monocyclic or 7-10-membered bicyclic heterocyclic moiety that is either saturated or partially unsaturated, and having, in addition to carbon atoms, one or more, preferably one to four, heteroatoms, as defined above. When used in reference to a ring atom of a heterocycle, the term “nitrogen” includes a substituted nitrogen. As an example, in a saturated or partially unsaturated ring having 0-3 heteroatoms selected from oxygen, sulfur or nitrogen, the nitrogen is N (as in 3,4-dihydro-2H-pyrrolyl), NH (as in pyrrolidinyl), or ⁺NR (as in N-substituted pyrrolidinyl).

A heterocyclic ring can be attached to its pendant group at any heteroatom or carbon atom that results in a stable structure and any of the ring atoms can be optionally substituted. Examples of such saturated or partially unsaturated heterocyclic radicals include, without limitation, tetrahydrofuranyl, tetrahydropyranyl, tetrahydrothiophenyl pyrrolidinyl, piperidinyl, pyrrolinyl, morpholinyl, tetrahydroquinolinyl, tetrahydroisoquinolinyl, decahydroquinolinyl, oxazolidinyl, piperazinyl, dioxanyl, dioxolanyl, diazepinyl, oxazepinyl, thiazepinyl, morpholinyl, and quinuclidinyl. The terms “heterocycle”, “heterocyclyl”, “heterocyclyl ring”, “heterocyclic group”, “heterocyclic moiety”, and “heterocyclic radical”, are used interchangeably herein, and also include groups in which a heterocyclyl ring is fused to one or more aryl, heteroaryl, or cycloaliphatic rings, such as indolinyl, 3H-indolyl, chromanyl, phenanthridinyl, or tetrahydroquinolinyl, where the radical or point of attachment is on the heterocyclyl ring. A heterocyclyl group is optionally mono- or bicyclic. The term “heterocyclylalkyl” refers to an alkyl group substituted by a heterocyclyl, wherein the alkyl and heterocyclyl portions independently are optionally substituted.

As used herein, the term “partially unsaturated” refers to a ring moiety that includes at least one double or triple bond. The term “partially unsaturated” is intended to encompass rings having multiple sites of unsaturation, but is not intended to include aryl or heteroaryl moieties, as herein defined.

As described herein, certain compounds of the invention contain “optionally substituted” moieties. In general, the term “substituted”, whether preceded by the term “optionally” or not, means that one or more hydrogens of the designated moiety are replaced with a suitable substituent. “Substituted” applies to one or more hydrogens that are either explicit or implicit from the structure (e.g.,

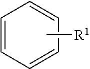

refers to at least

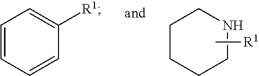

refers to at least

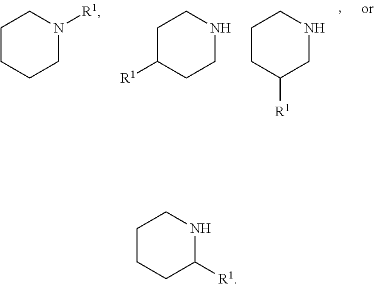

Unless otherwise indicated, an “optionally substituted” group has a suitable substituent at each substitutable position of the group, and when more than one position in any given structure is substituted with more than one substituent selected from a specified group, the substituent is either the same or different at every position. Combinations of substituents envisioned by this invention are preferably those that result in the formation of stable or chemically feasible compounds. The term “stable”, as used herein, refers to compounds that are not substantially altered when subjected to conditions to allow for their production, detection, and, in certain embodiments, their recovery, purification, and use for one or more of the purposes disclosed herein. Suitable monovalent substituents on a substitutable carbon atom of an “optionally substituted” group are independently deuterium; halogen; —(CH₂)₀₋₄R^{◯}; —(CH₂)₀₋₄OR^{◯}; —O(CH₂)₀₋₄R^{◯}, —O—(CH₂)₀₋₄C(O)OR^{◯}, —(CH₂)₀₋₄CH(OR^{◯})₂; —(CH₂)₀₋₄SR^{◯}; —(CH₂)₀₋₄Ph, which are optionally substituted with R^{◯}—(CH₂)₀₋₄O(CH₂)₀₋₁Ph, which is optionally substituted with R^{◯}—CH═CH Ph, which is optionally substituted with R^{◯}—(CH₂)₀₋₄O(CH₂)₀₋₁-pyridyl which is optionally substituted with R^{◯}—NO₂; —CN; —N₃; —(CH₂)₀₋₄N(R^{◯})₂; —(CH₂)₀₋₄N(R^{◯})C(O)R^{◯}; —N(R^{◯})C(S)R^{◯}; —(CH₂)₀₋₄N(R^{◯})C(O)NR^{◯}₂; —N(R^{◯})C(S)NR^{◯}₂; —(CH₂)₀₋₄N(R^{◯})C(O)OR^{◯}; —N(R^{◯})N(R^{◯})C(O)R^{◯}; —N(R^{◯})N(R^{◯})C(O)NR^{◯}₂; —N(R^{◯})N(R^{◯})C(O)OR^{◯}; —(CH₂)₀₋₄C(O)R^{◯}; —C(S)R^{◯}; —(CH₂)₀₋₄C(O)OR^{◯}; —(CH₂)₀₋₄C(O)SR^{◯}; —(CH₂)₀₋₄C(O)OSiR^{◯}₃; —(CH₂)₀₋₄OC(O)R^{◯}; —OC(O)(CH₂)₀₋₄SR^{◯}, SC(S)SR^{◯}, —(CH₂)₀₋₄SC(O)R^{◯}; —(CH₂)₀₋₄C(O)NR^{◯}₂; —C(S)NR^{◯}₂; —C(S)SR^{◯}; —SC(S)SR^{◯}, —(CH₂)₀₋₄OC(O)NR^{◯}₂; —C(O)N(OR^{◯})R^{◯}; —C(O)C(O)R^{◯}; —C(O)CH₂C(O)R^{◯}; —C(NOR^{◯})R^{◯}; —(CH₂)₀₋₄SSR^{◯}; —(CH₂)₀₋₄S(O)₂R^{◯}; —(CH₂)₀₋₄S(O)₂OR^{◯}; —(CH₂)₀₋₄OS(O)₂R^{◯}; —S(O)₂NR^{◯}₂; —(CH₂)₀₋₄S(O)R^{◯}; —N(R^{◯})S(O)₂NR^{◯}₂; —N(R^{◯})S(O)₂R; —N(OR^{◯})R^{◯}; —C(NH)NR^{◯}₂; —P(O)₂R^{◯}; —P(O)R^{◯}₂; —OP(O)R^{◯}₂; —OP(O)(OR^{◯})₂; SiR^{◯}₃; —(C₁₋₄ straight or branched alkylene)O—N(R^{◯})₂; or —(C₁₋₄ straight or branched alkylene)C(O)O—N(R^{◯})₂, wherein each R^{◯} is optionally substituted as defined below and is independently hydrogen, C₁₋₆ aliphatic, —CH₂Ph, —O(CH₂)₀₋₁Ph, —NH(CH₂)₀₋₁Ph, —CH₂-(5-6 membered heteroaryl ring), or a 5-6-membered saturated, partially unsaturated, or aryl ring having 0-4 heteroatoms independently selected from nitrogen, oxygen, or sulfur, or, notwithstanding the definition above, two independent occurrences of R^{◯}, taken together with their intervening atom(s), form a 3-12-membered saturated, partially unsaturated, or aryl mono- or bicyclic ring having 0-4 heteroatoms independently selected from nitrogen, oxygen, or sulfur, which is optionally substituted as defined below.

Suitable monovalent substituents on R^{◯} (or the ring formed by taking two independent occurrences of R^{◯} together with their intervening atoms), are independently deuterium, halogen, —(CH₂)₀₋₂R^{●}, -(haloR^{●}), —(CH₂)₀₋₂OH, —(CH₂)₀₋₂OR^{●}, —(CH₂)₀₋₂CH(OR^{●})₂; —O(haloR^{●}), —CN, —N₃, —(CH₂)₀₋₂C(O)R^{●}, —(CH₂)₀₋₂C(O)OH, —(CH₂)₀₋₂C(O)OR^{●}, —(CH₂)₀₋₂SR^{●}, —(CH₂)₀₋₂SH, —(CH₂)₀₋₂NH₂, —(CH₂)₀₋₂NHR^{●}, —(CH₂)₀₋₂NR^{●}₂, —NO₂, —SiR^{●}₃, —OSiR^{●}₃, —C(O)SR^{●}, —(C₁₋₄ straight or branched alkylene)C(O)OR^{●}, or —SSR^{●}, wherein each R^{●} is unsubstituted or where preceded by “halo” is substituted only with one or more halogens, and is independently selected from C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur. Suitable divalent substituents on a saturated carbon atom of R^{◯} include ═O and ═S.

Suitable divalent substituents on a saturated carbon atom of an “optionally substituted” group include the following: ═O, ═S, ═NNR*₂, ═NNHC(O)R*, ═NNHC(O)OR*, ═NNHS(O)₂R*, ═NR*, ═NOR*, —O(C(R*₂))₂₋₃O—, or —S(C(R*₂))₂₋₃S—, wherein each independent occurrence of R* is selected from hydrogen, C₁₋₆ aliphatic which is substituted as defined below, or an unsubstituted 5-6-membered saturated, partially unsaturated, or aryl ring having 0-4 heteroatoms independently selected from nitrogen, oxygen, or sulfur. Suitable divalent substituents that are bound to vicinal substitutable carbons of an “optionally substituted” group include: —O(CR*₂)₂₋₃O—, wherein each independent occurrence of R* is selected from hydrogen, C₁₋₆ aliphatic which is optionally substituted as defined below, or an unsubstituted 5-6-membered saturated, partially unsaturated, or aryl ring having 0-4 heteroatoms independently selected from nitrogen, oxygen, or sulfur.

Suitable substituents on the aliphatic group of R* include halogen, —R^{●}, -(haloR^{●}), —OH, —OR^{●}, —O(haloR^{●}), —CN, —C(O)OH, —C(O)OR^{●}, —NH₂, —NHR^{●}, —NR^{●}₂, or —NO₂, wherein each R^{●} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

Suitable substituents on a substitutable nitrogen of an “optionally substituted” group include —R^{†}, —NR^{†}₂, —C(O)R^{†}, —C(O)OR^{†}, —C(O)C(O)R^{†}, —C(O)CH₂C(O)R^{†}, —S(O)₂R^{†}, —S(O)₂NR^{†}₂, —C(S)NR^{†}₂, —C(NH)NR^{†}₂, or —N(R^{†})S(O)₂R^{†}; wherein each R^{†} is independently hydrogen, C₁₋₆ aliphatic which is optionally substituted as defined below, unsubstituted —OPh, or an unsubstituted 5-6-membered saturated, partially unsaturated, or aryl ring having 0-4 heteroatoms independently selected from nitrogen, oxygen, or sulfur, or, notwithstanding the definition above, two independent occurrences of R^{†}, taken together with their intervening atom(s) form an unsubstituted 3-12-membered saturated, partially unsaturated, or aryl mono- or bicyclic ring having 0-4 heteroatoms independently selected from nitrogen, oxygen, or sulfur.

Suitable substituents on the aliphatic group of R^{†} are independently halogen, —R^{●}, -(haloR^{●}), —OH, —OR^{●}, —O(haloR^{●}), —CN, —C(O)OH, —C(O)OR^{●}, —NH₂, —NHR^{●}, —NR^{●}₂, or —NO₂, wherein each R^{●} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

In certain embodiments, the terms “optionally substituted”, “optionally substituted alkyl,” “optionally substituted “optionally substituted alkenyl,” “optionally substituted alkynyl”, “optionally substituted carbocyclic,” “optionally substituted aryl”, “optionally substituted heteroaryl,” “optionally substituted heterocyclic,” and any other optionally substituted group as used herein, refer to groups that are substituted or unsubstituted by independent replacement of one, two, or three or more of the hydrogen atoms thereon with typical substituents including, but not limited to:
—F, —Cl, —Br, —I, deuterium,
—OH, protected hydroxy, alkoxy, oxo, thiooxo,
—NO₂, —CN, CF₃, N₃,
—NH₂, protected amino, —NH alkyl, —NH alkenyl, —NH alkynyl, —NH cycloalkyl, —NH-aryl, —NH-heteroaryl, —NH-heterocyclic, -dialkylamino, -diarylamino, -diheteroarylamino,
—O-alkyl, —O-alkenyl, —O-alkynyl, —O-cycloalkyl, —O-aryl, —O-heteroaryl, —O-heterocyclic,
—C(O)-alkyl, —C(O)-alkenyl, —C(O)-alkynyl, —C(O)-carbocyclyl, —C(O)-aryl, —C(O)-heteroaryl, —C(O)-heterocyclyl,
—CONH₂, —CONH-alkyl, —CONH-alkenyl, —CONH-alkynyl, —CONH-carbocyclyl, —CONH-aryl, —CONH-heteroaryl, —CONH-heterocyclyl,
—OCO₂-alkyl, —OCO₂-alkenyl, —OCO₂-alkynyl, —OCO₂-carbocyclyl, —OCO₂-aryl, —OCO₂-heteroaryl, —OCO₂-heterocyclyl, —OCONH₂, —OCONH-alkyl, —OCONH-alkenyl, —OONH-alkynyl, —OCONH-carbocyclyl, —OCONH-aryl, —OCONH-heteroaryl, —OCONH-heterocyclyl,
—NHC(O)-alkyl, —NHC(O)-alkenyl, —NHC(O)-alkynyl, —NHC(O)-carbocyclyl, —NHC(O)-aryl, —NHC(O)-heteroaryl, —NHC(O)-heterocyclyl, —NHCO₂-alkyl, —NHCO₂-alkenyl, —NHCO₂-alkynyl, —NHCO₂-carbocyclyl, —NHCO₂-aryl, —NHCO₂-heteroaryl, —NHCO₂-heterocyclyl, —NHC(O)NH₂, —NHC(O)NH-alkyl, —NHC(O)NH-alkenyl, —NHC(O)NH-alkenyl, —NHC(O)NH-carbocyclyl, —NHC(O)NH-aryl, —NHC(O)NH-heteroaryl, —NHC(O)NH-heterocyclyl, NHC(S)NH₂, —NHC(S)NH-alkyl, —NHC(S)NH-alkenyl, —NHC(S)NH-alkynyl, —NHC(S)NH-carbocyclyl, —NHC(S)NH-aryl, —NHC(S)NH-heteroaryl, —NHC(S)NH-heterocyclyl, —NHC(NH)NH₂, —NHC(NH)NH-alkyl, —NHC(NH)NH-alkenyl, —NHC(NH)NH-alkenyl, —NHC(NH)NH-carbocyclyl, —NHC(NH)NH-aryl, —NHC(NH)NH-heteroaryl, —NHC(NH)NH-heterocyclyl, —NHC(NH)-alkyl, —NHC(NH)-alkenyl, —NHC(NH)-alkenyl, —NHC(NH)-carbocyclyl, —NHC(NH)-aryl, —NHC(NH)-heteroaryl, —NHC(NH)-heterocyclyl,
—C(NH)NH-alkyl, —C(NH)NH-alkenyl, —C(NH)NH-alkynyl, —C(NH)NH-carbocyclyl, —C(NH)NH-aryl, —C(NH)NH-heteroaryl, —C(NH)NH-heterocyclyl,
—S(O)-alkyl, —S(O)-alkenyl, —S(O)-alkynyl, —S(O)-carbocyclyl, —S(O)-aryl, —S(O)-heteroaryl, —S(O)-heterocyclyl, —SO₂NH₂, —SO₂NH-alkyl, —SO₂NH-alkenyl, —SO₂NH-alkynyl, —SO₂NH-carbocyclyl, —SO₂NH-aryl, —SO₂NH-heteroaryl, —SO₂NH-heterocyclyl,
—NHSO₂-alkyl, —NHSO₂-alkenyl, —NHSO₂-alkynyl, —NHSO₂-carbocyclyl, —NHSO₂-aryl, —NHSO₂-heteroaryl, —NHSO₂-heterocyclyl,
—CH₂NH₂, —CH₂SO₂CH₃,
-mono-, di-, or tri-alkyl silyl,
-alkyl, -alkenyl, -alkynyl, -aryl, -arylalkyl, -heteroaryl, -heteroarylalkyl, -heterocycloalkyl, -cycloalkyl, -carbocyclic, -heterocyclic, polyalkoxyalkyl, polyalkoxy, -methoxymethoxy, -methoxyethoxy, —SH, —S-alkyl, —S-alkenyl, —S-alkynyl, —S-carbocyclyl, —S-aryl, —S-heteroaryl, —S-heterocyclyl, or methylthiomethyl.

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge et al., describe pharmaceutically acceptable salts in detail in J. Pharmaceutical Sciences, 1977, 66, 1-19, incorporated herein by reference. Pharmaceutically acceptable salts of the compounds of this invention include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, pivalate, propionate, stearate, succinate, sulfate, tartrate, thiocyanate, p-toluenesulfonate, undecanoate, valerate salts, and the like.

Salts derived from appropriate bases include alkali metal, alkaline earth metal, ammonium and N⁺(C₁₋₄alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, loweralkyl sulfonate and aryl sulfonate.

Unless otherwise stated, structures depicted herein are also meant to include all isomeric (e.g., enantiomeric, diastereomeric, tautomers, and geometric (or conformational)) forms of the structure; for example, the R and S configurations for each asymmetric center, Z and E double bond isomers, and Z and E conformational isomers. Therefore, single stereochemical isomers as well as enantiomeric, diastereomeric, and geometric (or conformational) mixtures of the present compounds are within the scope of the invention. Unless otherwise stated, all tautomeric forms of the compounds of the invention are within the scope of the invention.

Additionally, unless otherwise stated, structures depicted herein are also meant to include compounds that differ only in the presence of one or more isotopically enriched atoms. For example, compounds having the present structures including the replacement of hydrogen by deuterium or tritium, or the replacement of a carbon by a ¹³C- or ¹⁴C-enriched carbon are within the scope of this invention. In some embodiments, the group comprises one or more deuterium atoms.

There is furthermore intended that a compound of the formula I includes isotope-labeled forms thereof. An isotope-labeled form of a compound of the formula I is identical to this compound apart from the fact that one or more atoms of the compound have been replaced by an atom or atoms having an atomic mass or mass number which differs from the atomic mass or mass number of the atom which usually occurs naturally. Examples of isotopes which are readily commercially available and which can be incorporated into a compound of the formula I by well-known methods include isotopes of hydrogen, carbon, nitrogen, oxygen, phosphorus, fluorine and chlorine, for example ²H, ³H, ¹³C, ¹⁴C, ¹⁵N, ¹⁸O, ¹⁷O, ³¹P, ³²P, ³⁵S, ¹⁸F and ³⁶Cl, respectively.

A compound of the formula I, a prodrug, thereof or a pharmaceutically acceptable salt of either which contains one or more of the above-mentioned isotopes and/or other isotopes of other atoms is intended to be part of the present invention. An isotope-labeled compound of the formula I can be used in a number of beneficial ways. For example, an isotope-labeled compound of the formula I into which, for example, a radioisotope, such as ³H or ¹⁴C, has been incorporated, is suitable for medicament and/or substrate tissue distribution assays. These radioisotopes, i.e. tritium (³H) and carbon-14 (¹⁴C), are particularly preferred owing to simple preparation and excellent detectability. Incorporation of heavier isotopes, for example deuterium (²H), into a compound of the formula I has therapeutic advantages owing to the higher metabolic stability of this isotope-labeled compound. Higher metabolic stability translates directly into an increased in vivo half-life or lower dosages, which under most circumstances would represent a preferred embodiment of the present invention. An isotope-labeled compound of the formula I can usually be prepared by carrying out the procedures disclosed in the synthesis schemes and the related description, in the example part and in the preparation part in the present text, replacing a non-isotope-labeled reactant by a readily available isotope-labeled reactant. Compounds of the invention may be substituted by ¹⁸F, for use as PET imaging agents.

Deuterium (²H) can also be incorporated into a compound of the formula I for the purpose in order to manipulate the oxidative metabolism of the compound by way of the primary kinetic isotope effect. The primary kinetic isotope effect is a change of the rate for a chemical reaction that results from exchange of isotopic nuclei, which in turn is caused by the change in ground state energies necessary for covalent bond formation after this isotopic exchange. Exchange of a heavier isotope usually results in a lowering of the ground state energy for a chemical bond and thus causes a reduction in the rate in rate-limiting bond breakage. If the bond breakage occurs in or in the vicinity of a saddle-point region along the coordinate of a multi-product reaction, the product distribution ratios can be altered substantially. For explanation: if deuterium is bonded to a carbon atom at a non-exchangeable position, rate differences of k_{M}/k_{D}=2-7 are typical. If this rate difference is successfully applied to a com-pound of the formula I that is susceptible to oxidation, the profile of this compound in vivo can be drastically modified and result in improved pharmacokinetic properties.

When discovering and developing therapeutic agents, the person skilled in the art is able to optimize pharmacokinetic parameters while retaining desirable in vitro properties. It is reasonable to assume that many compounds with poor pharmacokinetic profiles are susceptible to oxidative metabolism. In vitro liver microsomal assays currently available provide valuable information on the course of oxidative metabolism of this type, which in turn permits the rational design of deuterated compounds of the formula I with improved stability through resistance to such oxidative metabolism. Significant improvements in the pharmacokinetic profiles of compounds of the formula I are thereby obtained, and can be expressed quantitatively in terms of increases in the in vivo half-life (t/2), concentration at maximum therapeutic effect (Cₘₐₓ), area under the dose response curve (AUC), and F; and in terms of reduced clearance, dose and materials costs.

The following is intended to illustrate the above: a compound of the formula I which has multiple potential sites of attack for oxidative metabolism, for example benzylic hydrogen atoms and hydrogen atoms bonded to a nitrogen atom, is prepared as a series of analogues in which various combinations of hydrogen atoms are replaced by deuterium atoms, so that some, most or all of these hydrogen atoms have been replaced by deuterium atoms. Half-life determinations enable favorable and accurate determination of the extent of the extent to which the improvement in resistance to oxidative metabolism has improved. In this way, it is determined that the half-life of the parent compound can be extended by up to 100% as the result of deuterium-hydrogen exchange of this type.

Deuterium-hydrogen exchange in a compound of the formula I can also be used to achieve a favorable modification of the metabolite spectrum of the starting compound in order to diminish or eliminate undesired toxic metabolites. For example, if a toxic metabolite arises through oxidative carbon-hydrogen (C—H) bond cleavage, it can reasonably be assumed that the deuterated analogue will greatly diminish or eliminate production of the unwanted metabolite, even if the particular oxidation is not a rate-determining step. Further information on the state of the art with respect to deuterium-hydrogen exchange may be found, for example in Hanzlik et al. (1990) J. Org. Chem. 55: 3992; Reider et al. (1987) J. Org. Chem. 52: 3326; Foster (1985) Adv. Drug Res. 14: 1; Gillette et al. (1994) Biochemistry 33(10): 2927; and Jarman et al. (1993) Carcinogenesis 16(4): 683.

As used herein, the term “modulator” is defined as a compound that binds to and/or inhibits the target with measurable affinity. In certain embodiments, a modulator has an IC₅₀ and/or binding constant of less about 50 M. In certain embodiments, a modulator has an IC₅₀ and/or binding constant of less than about 5 M. In certain embodiments, a modulator has an IC₅₀ and/or binding constant of between about 1 to about 5 M. In certain embodiments, a modulator has an IC₅₀ and/or binding constant of less than about 1 M. In certain embodiments, a modulator has an IC₅₀ and/or binding constant of between about 500 to about 1000 nM. In certain embodiments, a modulator has an IC₅₀ and/or binding constant of less than about 500 nM. In certain embodiments, a modulator has an IC₅₀ and/or binding constant of between about 100 to about 500 nM. In certain embodiments, a modulator has an IC₅₀ and/or binding constant of less than about 100 nM. In certain embodiments, a modulator has an IC₅₀ and/or binding constant of between about 10 to about 100 nM. In certain embodiments, a modulator has an IC₅₀ and/or binding constant of less than about 10 nM.

The terms “measurable affinity” and “measurably inhibit,” as used herein, means a measurable change in IDO activity between a sample comprising a compound of the present invention, or composition thereof, and IDO, and an equivalent sample comprising IDO, in the absence of said compound, or composition thereof.

Combinations of substituents and variables envisioned by this invention are only those that result in the formation of stable compounds. The term “stable”, as used herein, refers to compounds which possess stability sufficient to allow manufacture and which maintains the integrity of the compound for a sufficient period of time to be useful for the purposes detailed herein (e.g., therapeutic or prophylactic administration to a subject).

The recitation of a listing of chemical groups in any definition of a variable herein includes definitions of that variable as any single group or combination of listed groups. The recitation of an embodiment for a variable herein includes that embodiment as any single embodiment or in combination with any other embodiments or portions thereof.

According to one aspect, the present invention provides a compound of formula I,

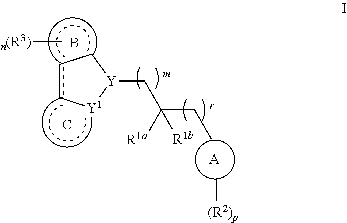

or a pharmaceutically acceptable salt thereof, wherein:
Y is CR or N;
Y¹ is C, CR, or N; wherein one of Y or Y¹ is N;
R^{1a} is —R, halogen, -haloalkyl, -hydroxyalkyl, —OR, —SR, —CN, —NO₂, —SO₂R, —SOR, —C(O)R, —CO₂R, —C(O)N(R)₂, —NRC(O)R, —NRC(O)N(R)₂, —NRSO₂R, or —N(R)₂;
R^{1b} is —R, halogen, -haloalkyl, -hydroxyalkyl, —OR, —SR, —CN, —NO₂, —SO₂R, —SOR, —C(O)R, —CO₂R, —C(O)N(R)₂, —NRC(O)R, —NRC(O)N(R)₂, —NRSO₂R, or —N(R)₂; or
R^{1a} and R^{1b}, together with the atom to which each is attached, may form a fused or spiro ring selected from C₅₋₁₀ aryl, a 3-8 membered saturated or partially unsaturated carbocyclic ring, a 3-7 membered heterocylic ring having 1-4 heteroatoms independently selected from nitrogen, oxygen, or sulfur, or a 5-6 membered monocyclic heteroaryl ring having 1-4 heteroatoms independently selected from nitrogen, oxygen, or sulfur; each of which is optionally substituted;
Ring A is C₅₋₁₀ aryl, a 3-8 membered saturated or partially unsaturated carbocyclic ring, a 3-7 membered heterocylic ring having 1-4 heteroatoms independently selected from nitrogen, oxygen, or sulfur, or a 5-6 membered monocyclic heteroaryl ring having 1-4 heteroatoms independently selected from nitrogen, oxygen, or sulfur;
each R² is independently —R, halogen, -haloalkyl, -hydroxyalkyl, —OR, —SR, —CN, —NO₂, —SO₂R, —SOR, —C(O)R, —CO₂R, —C(O)N(R)₂, —NRC(O)R, —NRC(O)N(R)₂, —NRSO₂R, or —N(R)₂;
Ring B is C₅₋₁₀ aryl, a 3-8 membered saturated or partially unsaturated carbocyclic ring, a 3-7 membered heterocylic ring having 1-3 heteroatoms independently selected from X¹, X², or X³, selected from nitrogen, oxygen, or sulfur, or a 5-6 membered monocyclic heteroaryl ring having 1-3 heteroatoms independently selected from X¹, X², or X³, each of which is selected from nitrogen, oxygen, or sulfur;
each R³ is independently —R, halogen, -haloalkyl, -hydroxyalkyl, —OR, —SR, —CN, —NO₂, —SO₂R, —SOR, —C(O)R, —CO₂R, —C(O)N(R)₂, —NRC(O)R, —NRC(O)N(R)₂, —NRSO₂R, or —N(R)₂;
Ring C is C₅₋₁₀ aryl, a 3-8 membered saturated or partially unsaturated carbocyclic ring, a 3-7 membered heterocylic ring having 1-4 heteroatoms independently selected from Z, Z¹, Z², Z³, or Z⁴, selected from nitrogen, oxygen, or sulfur, or a 5-6 membered monocyclic heteroaryl ring having 1-4 heteroatoms independently selected from Z, Z¹, Z², Z³, or Z⁴, each of which is selected from nitrogen, oxygen, or sulfur;
   each R is independently hydrogen, C₁₋₆ aliphatic, C₃₋₁₀ aryl, a 3-8 membered saturated or partially unsaturated carbocyclic ring, a 3-7 membered heterocylic ring having 1-4 heteroatoms independently selected from nitrogen, oxygen, or sulfur, or a 5-6 membered monocyclic heteroaryl ring having 1-4 heteroatoms independently selected from nitrogen, oxygen, or sulfur; each of which is optionally substituted; or
two R groups on the same atom are taken together with the atom to which they are attached to form a C₃₋₁₀ aryl, a 3-8 membered saturated or partially unsaturated carbocyclic ring, a 3-7 membered heterocylic ring having 1-4 heteroatoms independently selected from nitrogen, oxygen, or sulfur, or a 5-6 membered monocyclic heteroaryl ring having 1-4 heteroatoms independently selected from nitrogen, oxygen, or sulfur; each of which is optionally substituted;
m is 1 or 2;
n is 0, 1, 2, or 3;
p is 0, 1, 2, or 3; and
r is 0 or 1;
   wherein when Ring A is non-fluoro substituted cyclohexyl, Ring B is benzo, and Ring C is

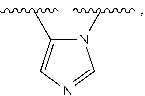 [SMILES: *c1cncn1*]

and R^{1a} is H, then R^{1b} cannot be OH.

In certain embodiments, Y is CR. In certain embodiments, Y is CH. In certain embodiments, Y is N.

In certain embodiments, Y¹ is CR. In certain embodiments, Y¹ is CH. In certain embodiments, Y¹ is C. In certain embodiments, Y¹ is N.

In certain embodiments, R^{1a} is —R.

In certain embodiments, R^{1a} is —H.

In certain embodiments, R^{1a} is halogen, -haloalkyl, -hydroxyalkyl, —OR, —SR, —CN, —NO₂, —SO₂R, —SOR, —C(O)R, —CO₂R, —C(O)N(R)₂, —NRC(O)R, —NRC(O)N(R)₂, —NRSO₂R, or —N(R)₂.

In certain embodiments, R^{1a} is halogen, -haloalkyl, -hydroxyalkyl, —OR, —NRC(O)R, —NRC(O)N(R)₂, —NRSO₂R, or —N(R)₂.

In certain embodiments, R^{1a} is halogen, —OR, —NRSO₂R, or —N(R)₂.

In certain embodiments, R^{1a} is

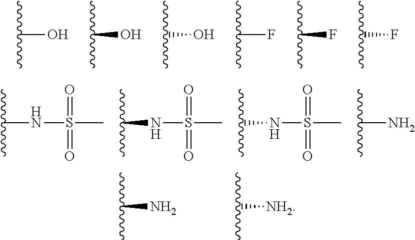

In certain embodiments, R^{1b} is —R.

In certain embodiments, R^{1b} is —H.

In certain embodiments, R^{1b} is halogen, -haloalkyl, -hydroxyalkyl, —OR, —SR, —CN, —NO₂, —SO₂R, —SOR, —C(O)R, —CO₂R, —C(O)N(R)₂, —NRC(O)R, —NRC(O)N(R)₂, —NRSO₂R, or —N(R)₂.

In certain embodiments, R^{1b} is halogen, -haloalkyl, -hydroxyalkyl, —OR, —NRC(O)R, —NRC(O)N(R)₂, —NRSO₂R, or —N(R)₂.

In certain embodiments, R^{1b} is halogen, —OR, —NRSO₂R, or —N(R)₂.

In certain embodiments, R^{1b} is

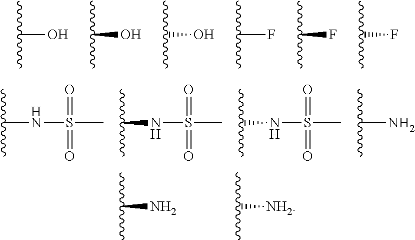

In certain embodiments, each R² is independently —R.

In certain embodiments, each R² is independently —H.

In certain embodiments, each R² is independently alkyl.

In certain embodiments, each R² is independently methyl, ethyl, propyl, i-propyl, n-butyl, s-butyl, t-butyl, straight chain or branched pentyl, or straight chain or branched hexyl.

In certain embodiments, each R² is independently halogen, -haloalkyl, -hydroxyalkyl, —OR, —SR, —CN, —NO₂, —SO₂R, —SOR, —C(O)R, —CO₂R, —C(O)N(R)₂, —NRC(O)R, —NRC(O)N(R)₂, —NRSO₂R, or —N(R)₂.

In certain embodiments, each R² is independently halogen or —OR.

In certain embodiments, each R² is independently —F or —OH.

In certain embodiments, two R² groups are R, and each R on the same atom are taken together with the atom to which they are attached to form a C₃₋₁₀ aryl, a 3-8 membered saturated or partially unsaturated carbocyclic ring, a 3-7 membered heterocylic ring having 1-4 heteroatoms independently selected from nitrogen, oxygen, or sulfur, or a 5-6 membered monocyclic heteroaryl ring having 1-4 heteroatoms independently selected from nitrogen, oxygen, or sulfur; each of which is optionally substituted. In certain embodiments, the ring is cyclopropyl, cyclobutyl, cyclopentyl, or cyclohexyl. In certain embodiments, the ring is cyclopropyl.

In certain embodiments, Ring A is C₅₋₁₀ aryl. In certain embodiments, Ring A is a 3-8 membered saturated or partially unsaturated carbocyclic ring. In certain embodiments, Ring A is a 3-7 membered heterocylic ring having 1-4 heteroatoms independently selected from nitrogen, oxygen, or sulfur. In certain embodiments, Ring A is a 5-6 membered monocyclic heteroaryl ring having 1-4 heteroatoms independently selected from nitrogen, oxygen, or sulfur.

In certain embodiments, Ring A is phenyl, cyclopropyl, cyclobutyl, cyclopentyl, cyclohexyl, cycloheptyl, adamantyl, cyclooctyl, [3.3.0]bicyclooctanyl, [4.3.0]bicyclononanyl, [4.4.0]bicyclodecanyl, [2.2.2]bicyclooctanyl, fluorenyl, indanyl, tetrahydronaphthyl, acridinyl, azocinyl, benzimidazolyl, benzofuranyl, benzothiofuranyl, benzothiophenyl, benzoxazolyl, benzthiazolyl, benztriazolyl, benztetrazolyl, benzisoxazolyl, benzisothiazolyl, benzimidazolinyl, carbazolyl, NH-carbazolyl, carbolinyl, chromanyl, chromenyl, cinnolinyl, decahydroquinolinyl, 2H,6H-1,5,2-dithiazinyl, dihydrofuro [2,3-b] tetrahydrofuran, furanyl, furazanyl, imidazolidinyl, imidazolinyl, imidazolyl, 1H-indazolyl, indolenyl, indolinyl, indolizinyl, indolyl, 3H-indolyl, isoindolinyl, isoindolenyl, isobenzofuranyl, isochromanyl, isoindazolyl, isoindolinyl, isoindolyl, isoquinolinyl, isothiazolyl, isoxazolyl, morpholinyl, naphthyridinyl, octahydroisoquinolinyl, oxadiazolyl, 1,2,3-oxadiazolyl, 1,2,4-oxadiazolyl; 1,2,5oxadiazolyl, 1,3,4-oxadiazolyl, oxazolidinyl, oxazolyl, oxazolidinyl, pyrimidinyl, phenanthridinyl, phenanthrolinyl, phenazinyl, phenothiazinyl, phenoxathiinyl, phenoxazinyl, phthalazinyl, piperazinyl, piperidinyl, pteridinyl, purinyl, pyranyl, pyrazinyl, pyrazolidinyl, pyrazolinyl, pyrazolyl, pyridazinyl, pyridooxazole, pyridoimidazole, pyridothiazole, pyridinyl, pyridyl, pyrimidinyl, pyrrolidinyl, pyrrolinyl, 2H-pyrrolyl, pyrrolyl, quinazolinyl, quinolinyl, 4H-quinolizinyl, quinoxalinyl, quinuclidinyl, tetrahydrofuranyl, tetrahydroisoquinolinyl, tetrahydroquinolinyl, 6H-1,2,5-thiadiazinyl, 1,2,3-thiadiazolyl, 1,2,4-thiadiazolyl, 1,2,5-thiadiazolyl, 1,3,4thiadiazolyl, thianthrenyl, thiazolyl, thienyl, thienothiazolyl, thienooxazolyl, thienoimidazolyl, thiophenyl, triazinyl, 1,2,3-triazolyl, 1,2,4-triazolyl, 1,2,5-triazolyl, 1,3,4-triazolyl, oxetanyl, azetidinyl, or xanthenyl.

In certain embodiments, Ring A is C-o aryl. In certain embodiments, Ring A is a 3-8 membered saturated or partially unsaturated carbocyclic ring.

In certain embodiments, Ring A is phenyl, piperidinyl, cycloheptyl, cyclohexyl, cyclopentyl, cyclobutyl, or cyclopropyl. In certain embodiments, Ring A is phenyl, piperidinyl, tetrahydropyran, or cyclohexyl.

In certain embodiments, Ring A is

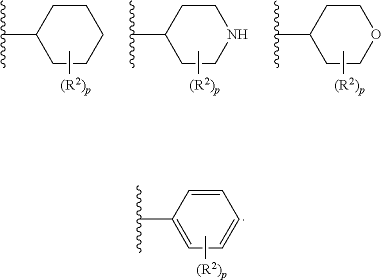

In certain embodiments, Ring A is

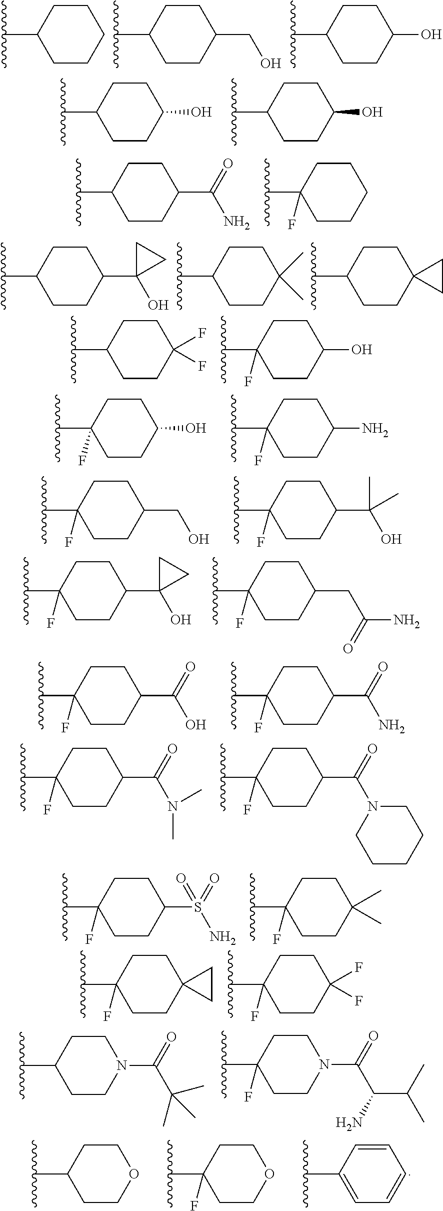

In certain embodiments, Ring B is C₅₋₁₀ aryl. In certain embodiments, Ring B is a 3-8 membered saturated or partially unsaturated carbocyclic ring. In certain embodiments, Ring B is a 3-7 membered heterocylic ring having 1-4 heteroatoms independently selected from nitrogen, oxygen, or sulfur. In certain embodiments, Ring B is a 5-6 membered monocyclic heteroaryl ring having 1-4 heteroatoms independently selected from nitrogen, oxygen, or sulfur.

In certain embodiments, Ring B is phenyl, cyclopropyl, cyclobutyl, cyclopentyl, cyclohexyl, cycloheptyl, adamantyl, cyclooctyl, [3.3.0]bicyclooctanyl, [4.3.0]bicyclononanyl, [4.4.0]bicyclodecanyl, [2.2.2]bicyclooctanyl, fluorenyl, indanyl, tetrahydronaphthyl, acridinyl, azocinyl, benzimidazolyl, benzofuranyl, benzothiofuranyl, benzothiophenyl, benzoxazolyl, benzthiazolyl, benztriazolyl, benztetrazolyl, benzisoxazolyl, benzisothiazolyl, benzimidazolinyl, carbazolyl, NH-carbazolyl, carbolinyl, chromanyl, chromenyl, cinnolinyl, decahydroquinolinyl, 2H,6H-1,5,2-dithiazinyl, dihydrofuro [2,3-b] tetrahydrofuran, furanyl, furazanyl, imidazolidinyl, imidazolinyl, imidazolyl, 1H-indazolyl, indolenyl, indolinyl, indolizinyl, indolyl, 3H-indolyl, isoindolinyl, isoindolenyl, isobenzofuranyl, isochromanyl, isoindazolyl, isoindolinyl, isoindolyl, isoquinolinyl, isothiazolyl, isoxazolyl, morpholinyl, naphthyridinyl, octahydroisoquinolinyl, oxadiazolyl, 1,2,3-oxadiazolyl, 1,2,4-oxadiazolyl; 1,2,5oxadiazolyl, 1,3,4-oxadiazolyl, oxazolidinyl, oxazolyl, oxazolidinyl, pyrimidinyl, phenanthridinyl, phenanthrolinyl, phenazinyl, phenothiazinyl, phenoxathiinyl, phenoxazinyl, phthalazinyl, piperazinyl, piperidinyl, pteridinyl, purinyl, pyranyl, pyrazinyl, pyrazolidinyl, pyrazolinyl, pyrazolyl, pyridazinyl, pyridooxazole, pyridoimidazole, pyridothiazole, pyridinyl, pyridyl, pyrimidinyl, pyrrolidinyl, pyrrolinyl, 2H-pyrrolyl, pyrrolyl, quinazolinyl, quinolinyl, 4H-quinolizinyl, quinoxalinyl, quinuclidinyl, tetrahydrofuranyl, tetrahydroisoquinolinyl, tetrahydroquinolinyl, 6H-1,2,5-thiadiazinyl, 1,2,3-thiadiazolyl, 1,2,4-thiadiazolyl, 1,2,5-thiadiazolyl, 1,3,4thiadiazolyl, thianthrenyl, thiazolyl, thienyl, thienothiazolyl, thienooxazolyl, thienoimidazolyl, thiophenyl, triazinyl, 1,2,3-triazolyl, 1,2,4-triazolyl, 1,2,5-triazolyl, 1,3,4-triazolyl, oxetanyl, azetidinyl, or xanthenyl.

In certain embodiments, Ring B is

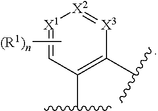

In certain embodiments, Ring B is phenyl, cycloheptyl, cyclohexyl, cyclopentyl, cyclobutyl, cyclopropyl, cyclohexadiene, pyridinyl, pyrimidinyl.

In certain embodiments, Ring B is

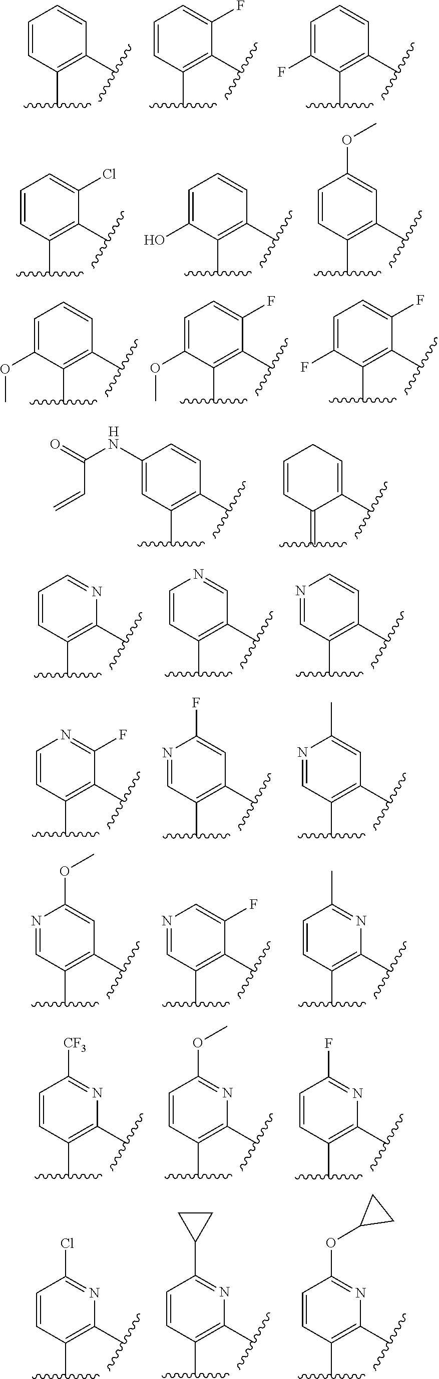

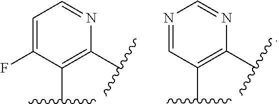 [SMILES: *c1nccc(F)c1* ; *c1cncnc1*]

In certain embodiments, Ring C is C₅₋₁₀ aryl. In certain embodiments, Ring C is a 3-8 membered saturated or partially unsaturated carbocyclic ring. In certain embodiments, Ring C is a 3-7 membered heterocylic ring having 1-4 heteroatoms independently selected from nitrogen, oxygen, or sulfur. In certain embodiments, Ring C is a 5-6 membered monocyclic heteroaryl ring having 1-4 heteroatoms independently selected from nitrogen, oxygen, or sulfur.

In certain embodiments, Ring C is phenyl, cyclopropyl, cyclobutyl, cyclopentyl, cyclohexyl, cycloheptyl, adamantyl, cyclooctyl, [3.3.0]bicyclooctanyl, [4.3.0]bicyclononanyl, [4.4.0]bicyclodecanyl, [2.2.2]bicyclooctanyl, fluorenyl, indanyl, tetrahydronaphthyl, acridinyl, azocinyl, benzimidazolyl, benzofuranyl, benzothiofuranyl, benzothiophenyl, benzoxazolyl, benzthiazolyl, benztriazolyl, benztetrazolyl, benzisoxazolyl, benzisothiazolyl, benzimidazolinyl, carbazolyl, NH-carbazolyl, carbolinyl, chromanyl, chromenyl, cinnolinyl, decahydroquinolinyl, 2H,6H-1,5,2-dithiazinyl, dihydrofuro [2,3-b] tetrahydrofuran, furanyl, furazanyl, imidazolidinyl, imidazolinyl, imidazolyl, 1H-indazolyl, indolenyl, indolinyl, indolizinyl, indolyl, 3H-indolyl, isoindolinyl, isoindolenyl, isobenzofuranyl, isochromanyl, isoindazolyl, isoindolinyl, isoindolyl, isoquinolinyl, isothiazolyl, isoxazolyl, morpholinyl, naphthyridinyl, octahydroisoquinolinyl, oxadiazolyl, 1,2,3-oxadiazolyl, 1,2,4-oxadiazolyl; 1,2,5oxadiazolyl, 1,3,4-oxadiazolyl, oxazolidinyl, oxazolyl, oxazolidinyl, pyrimidinyl, phenanthridinyl, phenanthrolinyl, phenazinyl, phenothiazinyl, phenoxathiinyl, phenoxazinyl, phthalazinyl, piperazinyl, piperidinyl, pteridinyl, purinyl, pyranyl, pyrazinyl, pyrazolidinyl, pyrazolinyl, pyrazolyl, pyridazinyl, pyridooxazole, pyridoimidazole, pyridothiazole, pyridinyl, pyridyl, pyrimidinyl, pyrrolidinyl, pyrrolinyl, 2H-pyrrolyl, pyrrolyl, quinazolinyl, quinolinyl, 4H-quinolizinyl, quinoxalinyl, quinuclidinyl, tetrahydrofuranyl, tetrahydroisoquinolinyl, tetrahydroquinolinyl, 6H-1,2,5-thiadiazinyl, 1,2,3-thiadiazolyl, 1,2,4-thiadiazolyl, 1,2,5-thiadiazolyl, 1,3,4thiadiazolyl, thianthrenyl, thiazolyl, thienyl, thienothiazolyl, thienooxazolyl, thienoimidazolyl, thiophenyl, triazinyl, 1,2,3-triazolyl, 1,2,4-triazolyl, 1,2,5-triazolyl, 1,3,4-triazolyl, oxetanyl, azetidinyl, or xanthenyl.

In certain embodiments, Ring C is

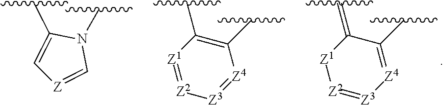

In certain embodiments, Ring C is phenyl, imidazole, pyrrole, pyridine, pyrimidine, pyrazine, pyridazine, dihydropyridine, dihydropyrimidine, dihydropyrazine, or dihydropyridazine.

In certain embodiments, Ring C is

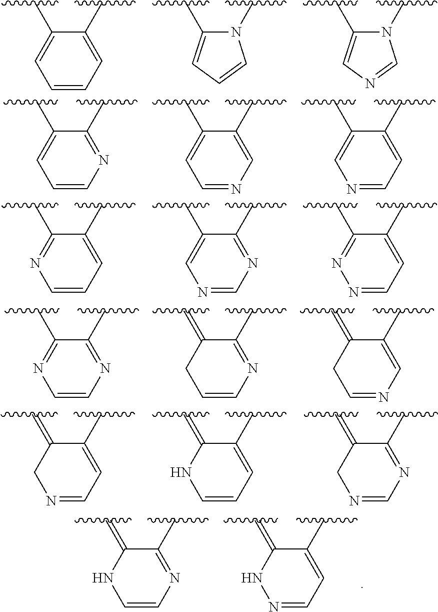

In certain embodiments, the present invention provides a compound of formula II,

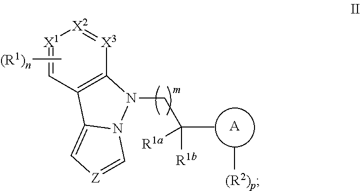

or a pharmaceutically acceptable salt thereof, wherein each of Ring A, X¹, X², X³, Z, R, R^{1a}, R^{1b}R², R³, m, n, and p, is as defined above and described in embodiments, classes and subclasses above and herein, singly or in combination.

In certain embodiments, the present invention provides a compound of formula Ill:

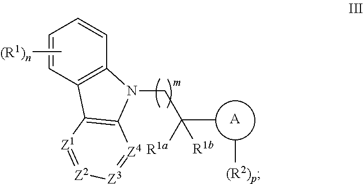

or a pharmaceutically acceptable salt thereof, wherein each of Ring A, Z¹, Z², Z³, Z⁴, R, R^{1a}, R^{1b}, R², R³, m, n, and p, is as defined above and described in embodiments, classes and subclasses above and herein, singly or in combination.

In certain embodiments, the present invention provides a compound of formula IV:

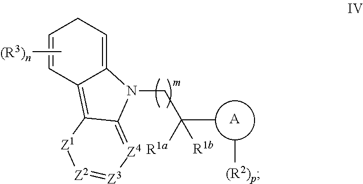

or a pharmaceutically acceptable salt thereof, wherein each of Ring A, Z¹, Z², Z³, Z⁴, R, R^{1a}, R^{1b}, R², R³, m, n, and p, is as defined above and described in embodiments, classes and subclasses above and herein, singly or in combination.

In certain embodiments, the present invention provides a compound of any of the formulae presented below, or a pharmaceutically acceptable salt thereof, wherein each of Ring A, X¹, X², X³, Z, Z¹, Z², Z³, Z⁴, R, R^{1a}, R^{1b}, R², R³, m, n, and p, is as defined above and described in embodiments, classes and subclasses above and herein, singly or in combination.

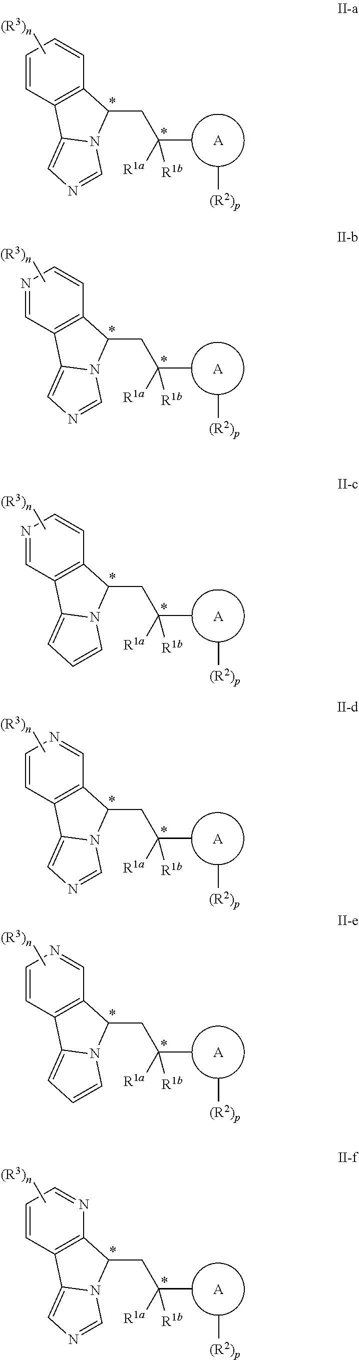

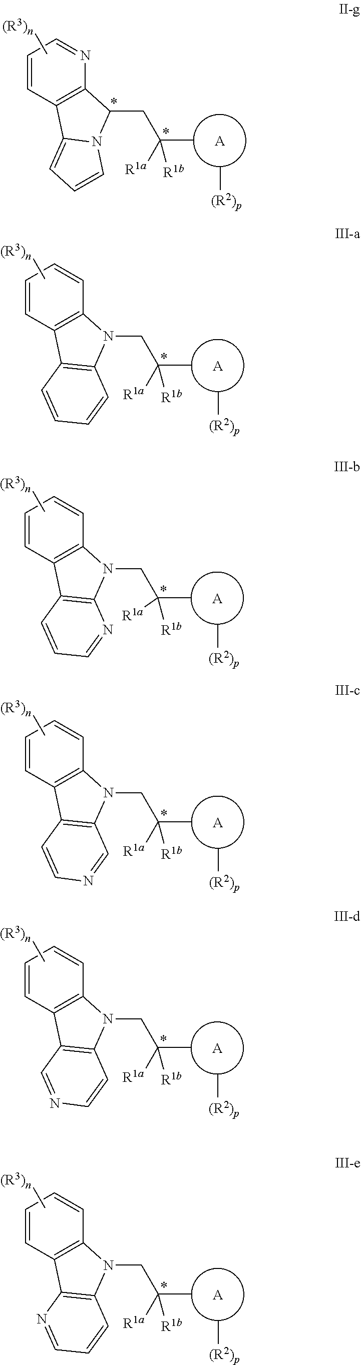

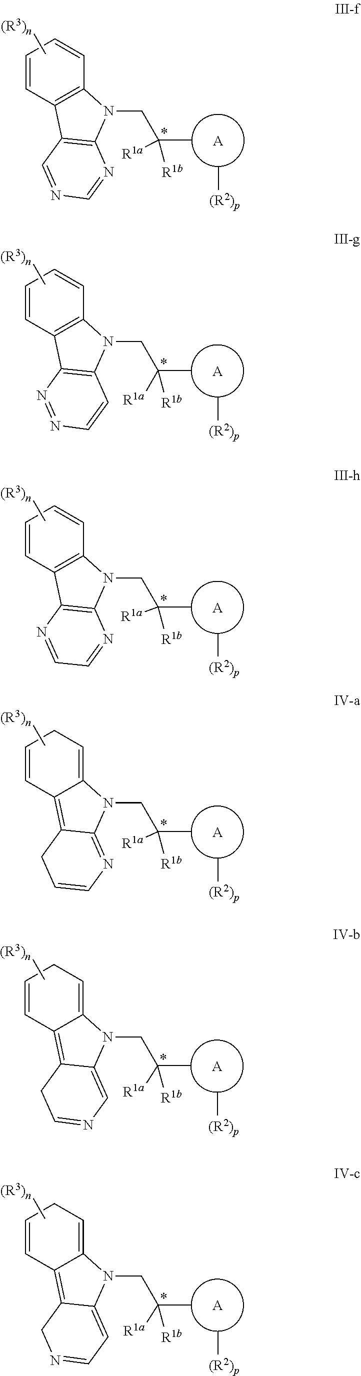

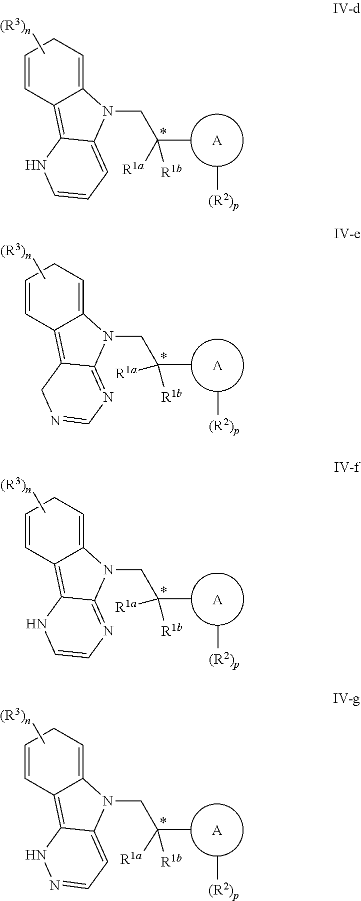

In certain embodiments, the present invention provides a compound of formula V:

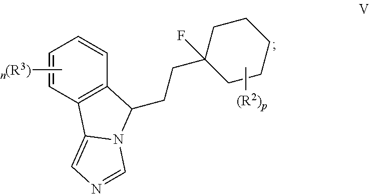

or a pharmaceutically acceptable salt thereof, wherein each of R², R³, n, and p, is as defined above and described in embodiments, classes and subclasses above and herein, singly or in combination.

In certain embodiments, the present invention provides a compound of formula VI:

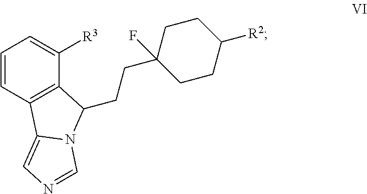

or a pharmaceutically acceptable salt thereof, wherein each of R² and R³ is as defined above and described in embodiments, classes and subclasses above and herein, singly or in combination.

In certain embodiments, the compounds embodied by the invention include the racemate of *. In certain embodiments, the compounds embodied by the invention include the (R) enantiomer of *. In certain embodiments, the compounds embodied by the invention include the (S) enantiomer of *. In certain embodiments, each enantiomer is over 50% enantiopure. In certain embodiments, each enantiomer is over 75% enantiopure. In certain embodiments, each enantiomer is over 90% enantiopure. In certain embodiments, each enantiomer is over 50% enantiopure. In certain embodiments, each enantiomer is over 95% enantiopure. In certain embodiments, each enantiomer is over 97% enantiopure. In certain embodiments, each enantiomer is over 99% enantiopure.

In certain embodiments, when two stereocenters in a compound exist, the invention includes each diastereomer, and each enantiomer of each diastereomer (e.g., (R)(R), (R)(S), (S)(R), and (S)(S)).

In certain embodiments, when three stereocenters in a compound exist, the invention includes each diastereomer, and each enantiomer of each diastereomer (e.g., (R)(R)(R), (R)(S)(R), (R)(R)(S), (S)(R)(R), (S)(R)(S), (R)(S)(S), (S)(S)(R), and (S)(S)(S)).

In certain embodiments, the invention provides a compound of any of the formulae presented herein, wherein each of Ring A, Ring B, Ring C, Y, Y¹, R^{1a}, R^{1b}, R², R³, X¹, X², X³, Z, Z¹, Z², Z³, Z⁴, R, m, n, and p, is as defined above and described in embodiments, classes and subclasses above and herein, singly or in combination.

In certain embodiments, the invention provides a compound selected from Table 1:

TABLE 1
Selected IDO1 inhibitors

| | |
|---|---|
| 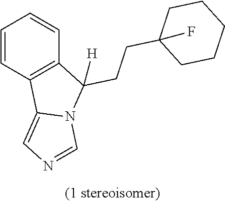 | 1a |
| 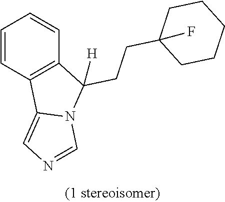 | 1b |
| 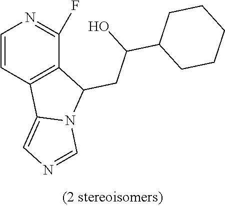 [SMILES: OC(CC1c2c(ccnc2F)-c2cncn21)C1CCCCC1] | 2a |
| 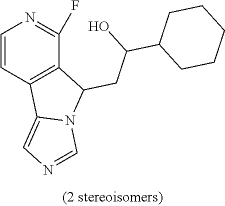 [SMILES: OC(CC1c2c(ccnc2F)-c2cncn21)C1CCCCC1] | 2b |
| 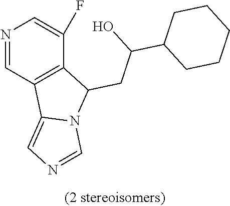 [SMILES: OC(CC1c2c(F)cncc2-c2cncn21)C1CCCCC1] | 3a |
| 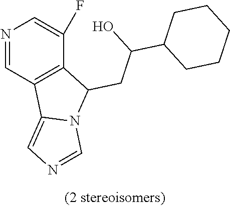 [SMILES: OC(CC1c2c(F)cncc2-c2cncn21)C1CCCCC1] | 3b |
| 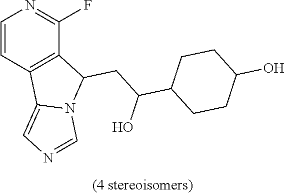 [SMILES: OC1CCC(C(O)CC2c3c(ccnc3F)-c3cncn32)CC1] | 4a |
| 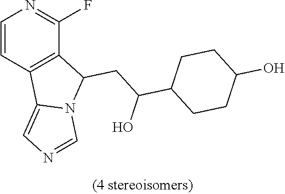 [SMILES: OC1CCC(C(O)CC2c3c(ccnc3F)-c3cncn32)CC1] | 4b |
| 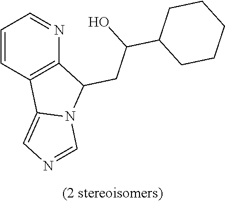 [SMILES: OC(CC1c2ncccc2-c2cncn21)C1CCCCC1] | 5a |
| 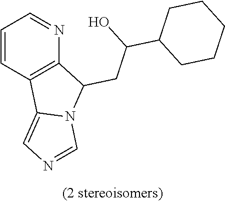 [SMILES: OC(CC1c2ncccc2-c2cncn21)C1CCCCC1] | 5b |
| 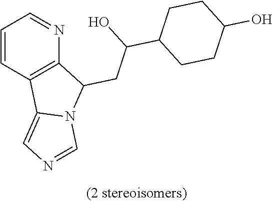 [SMILES: OC1CCC(C(O)CC2c3ncccc3-c3cncn32)CC1] | 6a |
| 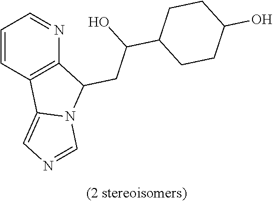 [SMILES: OC1CCC(C(O)CC2c3ncccc3-c3cncn32)CC1] | 6b |
| 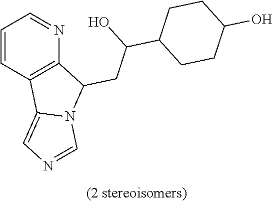 [SMILES: OC1CCC(C(O)CC2c3ncccc3-c3cncn32)CC1] | 6c |
| 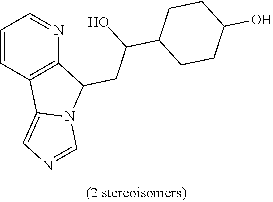 [SMILES: OC1CCC(C(O)CC2c3ncccc3-c3cncn32)CC1] | 6d |
| 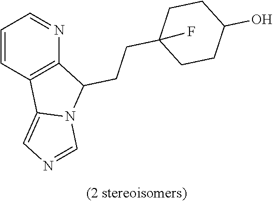 | 7a |
| 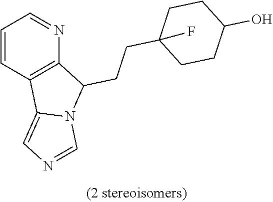 | 7b |
| 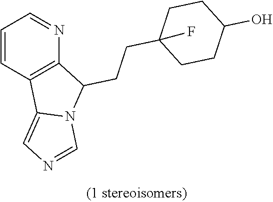 | 8a |
| 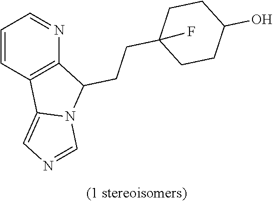 | 8b |
| 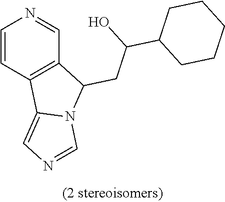 [SMILES: OC(CC1c2cnccc2-c2cncn21)C1CCCCC1] | 9a |
| 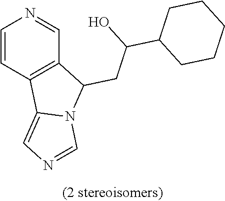 [SMILES: OC(CC1c2cnccc2-c2cncn21)C1CCCCC1] | 9b |
| 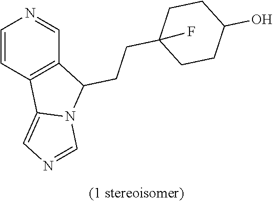 | 10a |
| 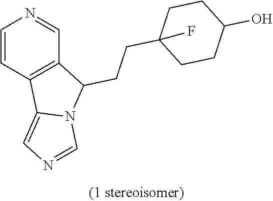 | 10b |
| 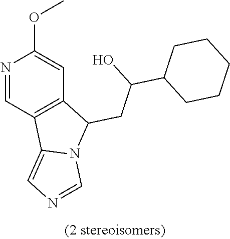 [SMILES: COc1cc2c(cn1)-c1cncn1C2CC(O)C1CCCCC1] | 11a |
| 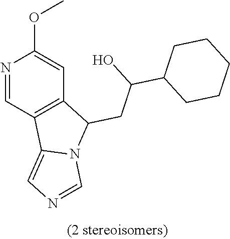 [SMILES: COc1cc2c(cn1)-c1cncn1C2CC(O)C1CCCCC1] | 11b |
| 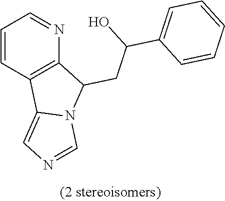 [SMILES: OC(CC1c2ncccc2-c2cncn21)c1ccccc1] | 12a |
| 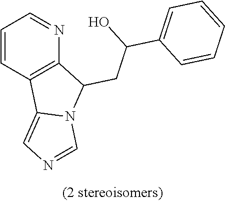 [SMILES: OC(CC1c2ncccc2-c2cncn21)c1ccccc1] | 12b |
| 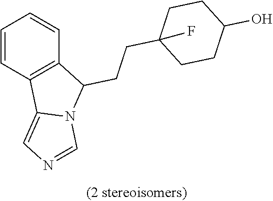 | 13a |
| 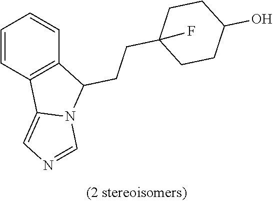 | 13b |
| 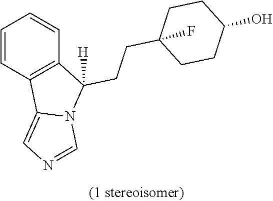 | 14a |
| 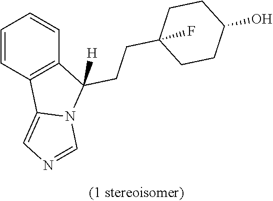 | 14b |
| 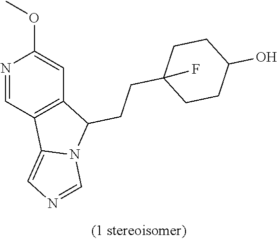 | 15a |
| 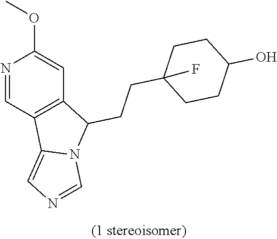 | 15b |
| 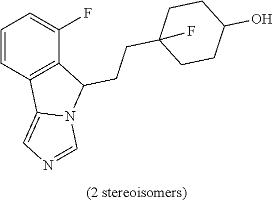 | 16a |
| 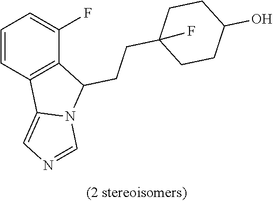 | 16b |
| 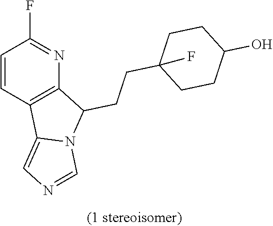 | 17 |
| 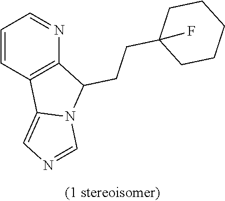 | 18a |
| 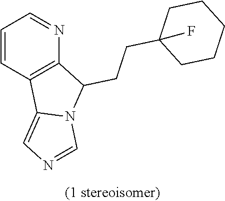 | 18b |
| 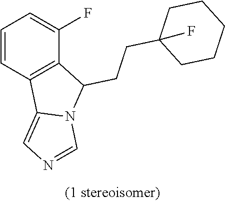 | 19a |
| 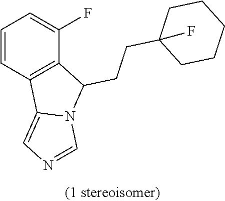 | 19b |
| 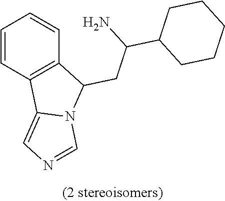 [SMILES: NC(CC1c2ccccc2-c2cncn21)C1CCCCC1] | 20a |
| 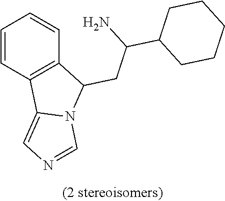 [SMILES: NC(CC1c2ccccc2-c2cncn21)C1CCCCC1] | 20b |
| 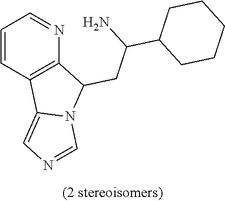 [SMILES: NC(CC1c2ncccc2-c2cncn21)C1CCCCC1] | 21a |
| 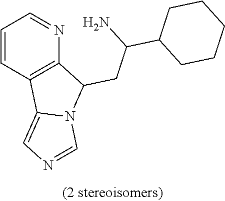 [SMILES: NC(CC1c2ncccc2-c2cncn21)C1CCCCC1] | 21b |
| 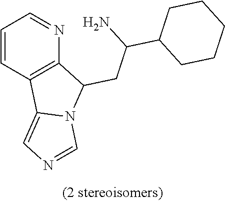 [SMILES: NC(CC1c2ncccc2-c2cncn21)C1CCCCC1] | 21c |
| 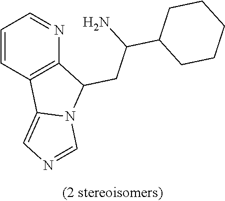 [SMILES: NC(CC1c2ncccc2-c2cncn21)C1CCCCC1] | 21d |
| 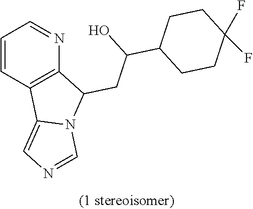 [SMILES: OC(CC1c2ncccc2-c2cncn21)C1CCC(F)(F)CC1] | 22a |
| 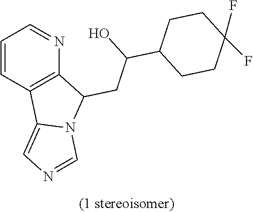 [SMILES: OC(CC1c2ncccc2-c2cncn21)C1CCC(F)(F)CC1] | 22b |
| 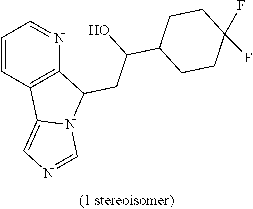 [SMILES: OC(CC1c2ncccc2-c2cncn21)C1CCC(F)(F)CC1] | 22c |
| 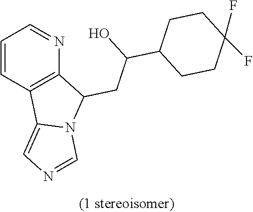 [SMILES: OC(CC1c2ncccc2-c2cncn21)C1CCC(F)(F)CC1] | 22d |
| 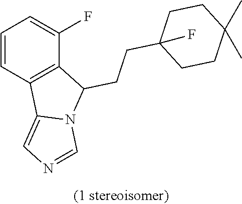 | 23a |
| 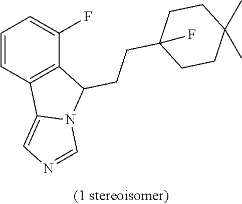 | 23b |
| 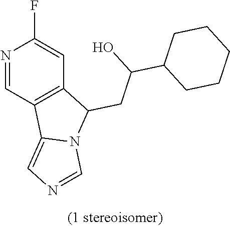 [SMILES: OC(CC1c2cc(F)ncc2-c2cncn21)C1CCCCC1] | 24a |
| 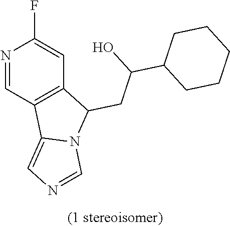 [SMILES: OC(CC1c2cc(F)ncc2-c2cncn21)C1CCCCC1] | 24b |
| 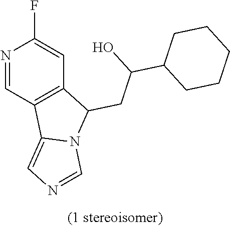 [SMILES: OC(CC1c2cc(F)ncc2-c2cncn21)C1CCCCC1] | 24c |
| 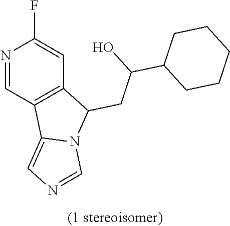 [SMILES: OC(CC1c2cc(F)ncc2-c2cncn21)C1CCCCC1] | 24d |
| 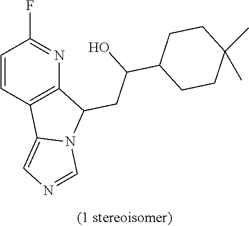 [SMILES: CC1(C)CCC(C(O)CC2c3nc(F)ccc3-c3cncn32)CC1] | 25a |
| 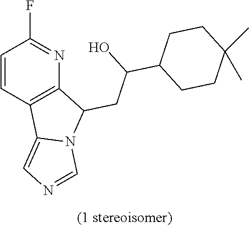 [SMILES: CC1(C)CCC(C(O)CC2c3nc(F)ccc3-c3cncn32)CC1] | 25b |
| 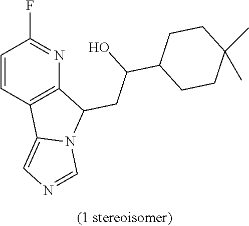 [SMILES: CC1(C)CCC(C(O)CC2c3nc(F)ccc3-c3cncn32)CC1] | 25c |
| 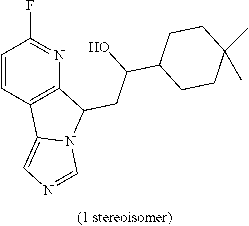 [SMILES: CC1(C)CCC(C(O)CC2c3nc(F)ccc3-c3cncn32)CC1] | 25d |
| 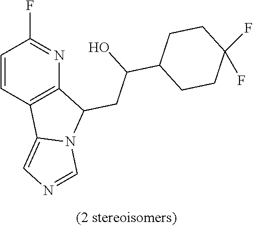 [SMILES: OC(CC1c2nc(F)ccc2-c2cncn21)C1CCC(F)(F)CC1] | 26a |
| 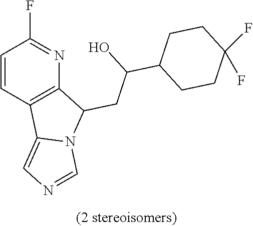 [SMILES: OC(CC1c2nc(F)ccc2-c2cncn21)C1CCC(F)(F)CC1] | 26b |
| 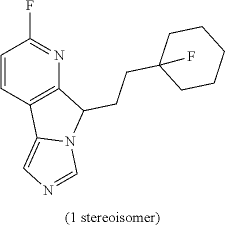 | 27a |
| 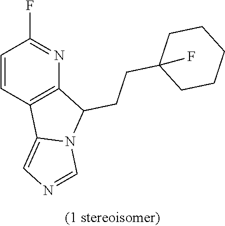 | 27b |
| 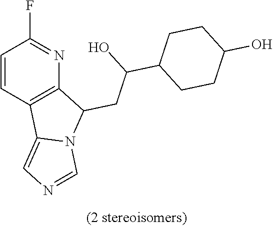 [SMILES: OC1CCC(C(O)CC2c3nc(F)ccc3-c3cncn32)CC1] | 28a |
| 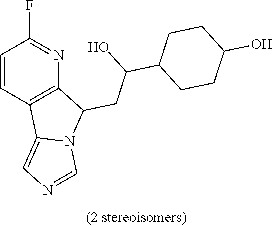 [SMILES: OC1CCC(C(O)CC2c3nc(F)ccc3-c3cncn32)CC1] | 28b |
| 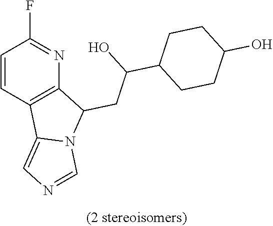 [SMILES: OC1CCC(C(O)CC2c3nc(F)ccc3-c3cncn32)CC1] | 28c |
| 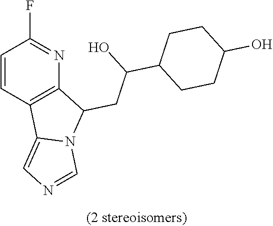 [SMILES: OC1CCC(C(O)CC2c3nc(F)ccc3-c3cncn32)CC1] | 28d |
| 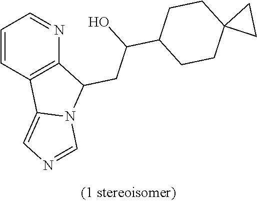 [SMILES: OC(CC1c2ncccc2-c2cncn21)C1CCC2(CC1)CC2] | 29a |
| 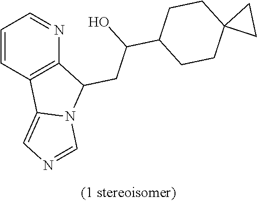 [SMILES: OC(CC1c2ncccc2-c2cncn21)C1CCC2(CC1)CC2] | 29b |
| 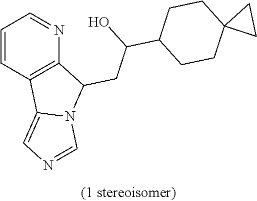 [SMILES: OC(CC1c2ncccc2-c2cncn21)C1CCC2(CC1)CC2] | 29c |
| 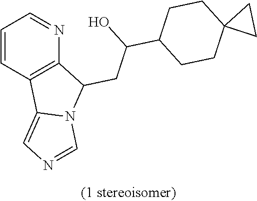 [SMILES: OC(CC1c2ncccc2-c2cncn21)C1CCC2(CC1)CC2] | 29d |
| 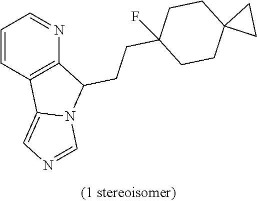 [SMILES: FC1(CCC2c3ncccc3-c3cncn32)CCC2(CC1)CC2] | 30a |
| 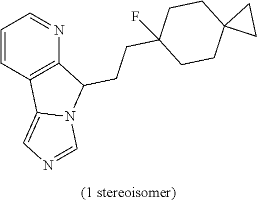 [SMILES: FC1(CCC2c3ncccc3-c3cncn32)CCC2(CC1)CC2] | 30b |
| 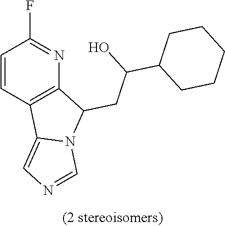 [SMILES: OC(CC1c2nc(F)ccc2-c2cncn21)C1CCCCC1] | 31a |
| 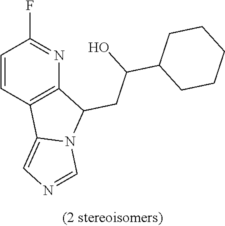 [SMILES: OC(CC1c2nc(F)ccc2-c2cncn21)C1CCCCC1] | 31b |
| 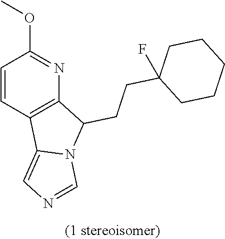 [SMILES: COc1ccc2c(n1)C(CCC1(F)CCCCC1)n1cncc1-2] | 32b |
| 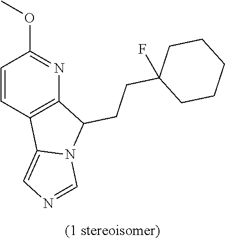 [SMILES: COc1ccc2c(n1)C(CCC1(F)CCCCC1)n1cncc1-2] | 32c |
| 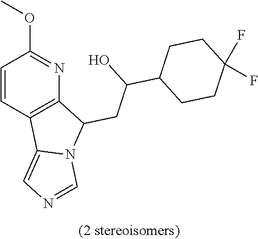 [SMILES: COc1ccc2c(n1)C(CC(O)C1CCC(F)(F)CC1)n1cncc1-2] | 33a |
| 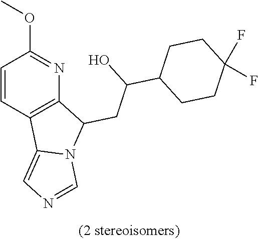 [SMILES: COc1ccc2c(n1)C(CC(O)C1CCC(F)(F)CC1)n1cncc1-2] | 33b |
| 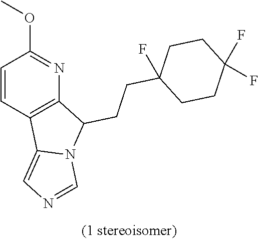 [SMILES: COc1ccc2c(n1)C(CCC1(F)CCC(F)(F)CC1)n1cncc1-2] | 34a |
| 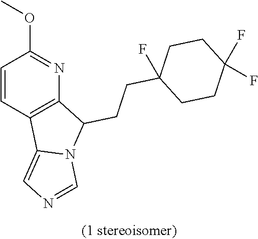 [SMILES: COc1ccc2c(n1)C(CCC1(F)CCC(F)(F)CC1)n1cncc1-2] | 34b |
| 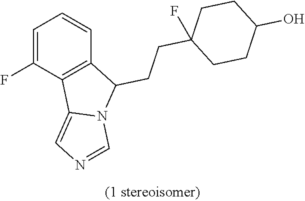 [SMILES: OC1CCC(F)(CCC2c3cccc(F)c3-c3cncn32)CC1] | 35a |
| 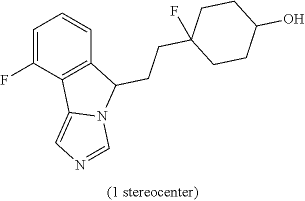 [SMILES: OC1CCC(F)(CCC2c3cccc(F)c3-c3cncn32)CC1] | 35b |
| 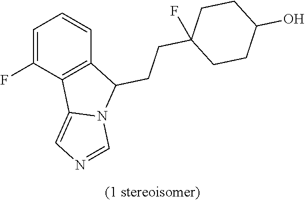 [SMILES: OC1CCC(F)(CCC2c3cccc(F)c3-c3cncn32)CC1] | 35c |
| 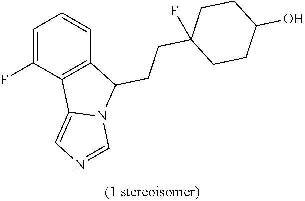 [SMILES: OC1CCC(F)(CCC2c3cccc(F)c3-c3cncn32)CC1] | 35d |
| 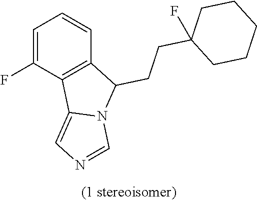 [SMILES: Fc1cccc2c1-c1cncn1C2CCC1(F)CCCCC1] | 36a |
| 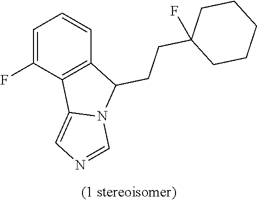 [SMILES: Fc1cccc2c1-c1cncn1C2CCC1(F)CCCCC1] | 36b |
| 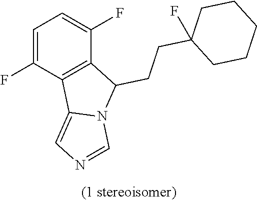 [SMILES: Fc1ccc(F)c2c1-c1cncn1C2CCC1(F)CCCCC1] | 37a |
| 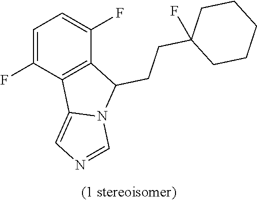 [SMILES: Fc1ccc(F)c2c1-c1cncn1C2CCC1(F)CCCCC1] | 37b |
| 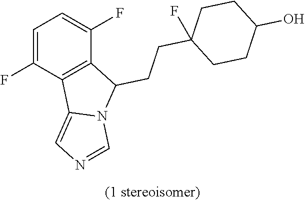 [SMILES: OC1CCC(F)(CCC2c3c(F)ccc(F)c3-c3cncn32)CC1] | 38a |
| 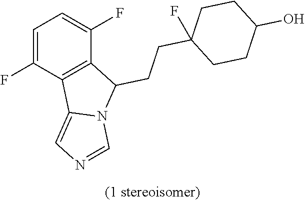 [SMILES: OC1CCC(F)(CCC2c3c(F)ccc(F)c3-c3cncn32)CC1] | 38b |
| 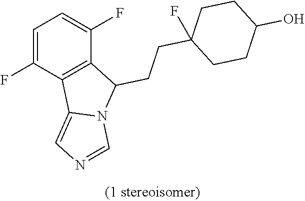 [SMILES: OC1CCC(F)(CCC2c3c(F)ccc(F)c3-c3cncn32)CC1] | 38c |
| 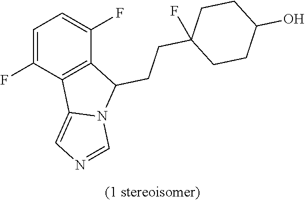 [SMILES: OC1CCC(F)(CCC2c3c(F)ccc(F)c3-c3cncn32)CC1] | 38d |
| 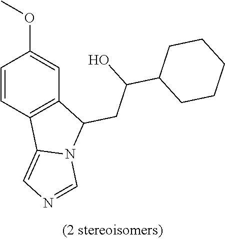 [SMILES: COc1ccc2c(c1)C(CC(O)C1CCCCC1)n1cncc1-2] | 39a |
| 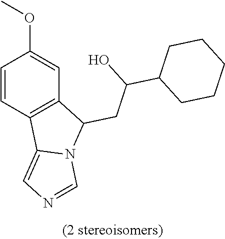 [SMILES: COc1ccc2c(c1)C(CC(O)C1CCCCC1)n1cncc1-2] | 39b |
| 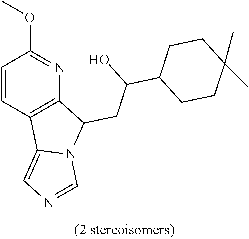 [SMILES: COc1ccc2c(n1)C(CC(O)C1CCC(C)(C)CC1)n1cncc1-2] | 40a |
| 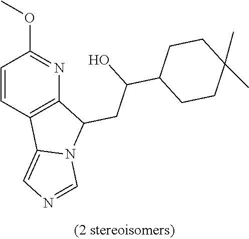 [SMILES: COc1ccc2c(n1)C(CC(O)C1CCC(C)(C)CC1)n1cncc1-2] | 40b |
| 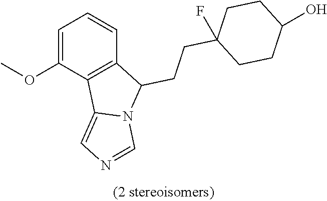 [SMILES: COc1cccc2c1-c1cncn1C2CCC1(F)CCC(O)CC1] | 41a |
| 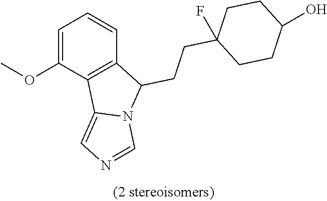 [SMILES: COc1cccc2c1-c1cncn1C2CCC1(F)CCC(O)CC1] | 41b |
| 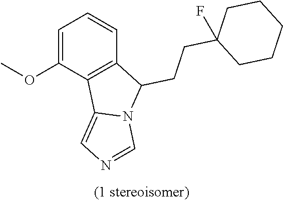 [SMILES: COc1cccc2c1-c1cncn1C2CCC1(F)CCCCC1] | 42a |
| 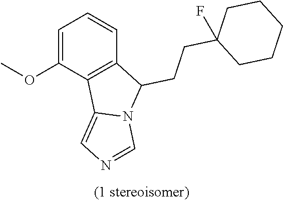 [SMILES: COc1cccc2c1-c1cncn1C2CCC1(F)CCCCC1] | 42b |
| 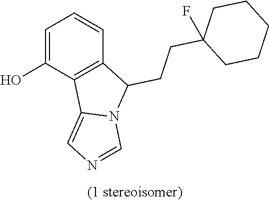 [SMILES: Oc1cccc2c1-c1cncn1C2CCC1(F)CCCCC1] | 43a |
| 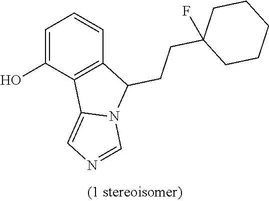 [SMILES: Oc1cccc2c1-c1cncn1C2CCC1(F)CCCCC1] | 43b |
| 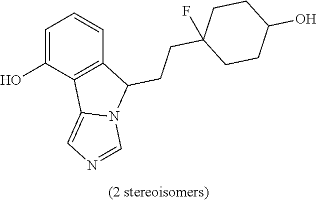 [SMILES: Oc1cccc2c1-c1cncn1C2CCC1(F)CCC(O)CC1] | 44a |
| 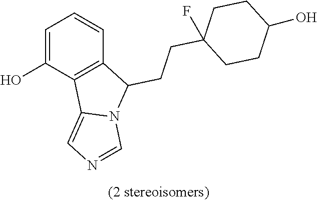 [SMILES: Oc1cccc2c1-c1cncn1C2CCC1(F)CCC(O)CC1] | 44b |
| 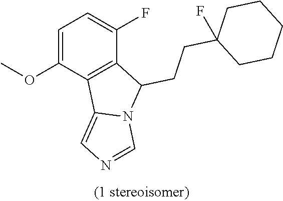 [SMILES: COc1ccc(F)c2c1-c1cncn1C2CCC1(F)CCCCC1] | 45a |
| 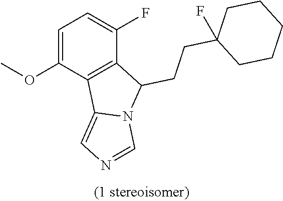 [SMILES: COc1ccc(F)c2c1-c1cncn1C2CCC1(F)CCCCC1] | 45b |
| 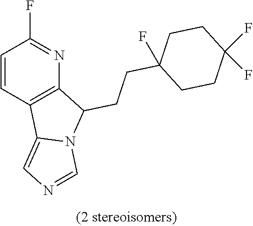 [SMILES: Fc1ccc2c(n1)C(CCC1(F)CCC(F)(F)CC1)n1cncc1-2] | 46 |
| 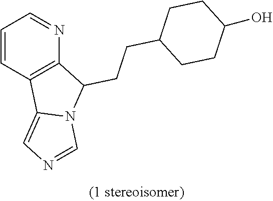 [SMILES: OC1CCC(CCC2c3ncccc3-c3cncn32)CC1] | 47 |
| 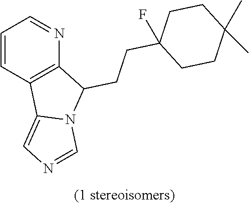 [SMILES: CC1(C)CCC(F)(CCC2c3ncccc3-c3cncn32)CC1] | 48a |
| 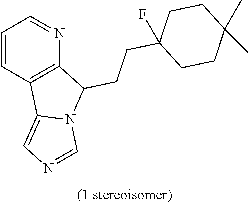 [SMILES: CC1(C)CCC(F)(CCC2c3ncccc3-c3cncn32)CC1] | 48b |
| 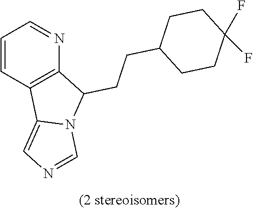 [SMILES: FC1(F)CCC(CCC2c3ncccc3-c3cncn32)CC1] | 49 |
| 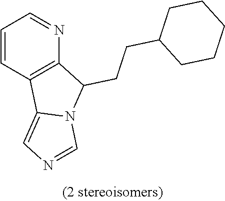 [SMILES: c1cnc2c(c1)-c1cncn1C2CCC1CCCCC1] | 50 |
| 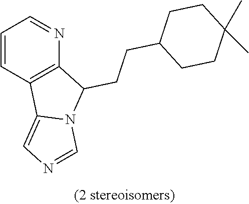 [SMILES: CC1(C)CCC(CCC2c3ncccc3-c3cncn32)CC1] | 51 |
| 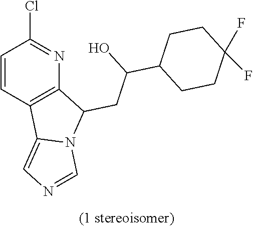 [SMILES: OC(CC1c2nc(Cl)ccc2-c2cncn21)C1CCC(F)(F)CC1] | 52a |
| 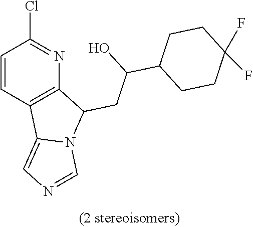 [SMILES: OC(CC1c2nc(Cl)ccc2-c2cncn21)C1CCC(F)(F)CC1] | 52b |
| 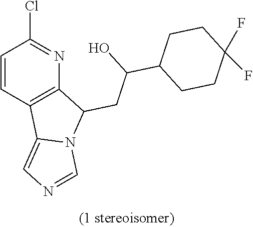 [SMILES: OC(CC1c2nc(Cl)ccc2-c2cncn21)C1CCC(F)(F)CC1] | 52c |
| 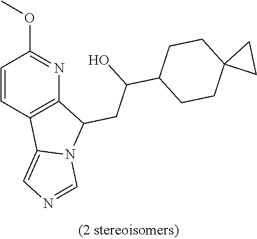 [SMILES: COc1ccc2c(n1)C(CC(O)C1CCC3(CC1)CC3)n1cncc1-2] | 53a |
| 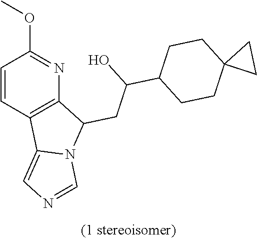 [SMILES: COc1ccc2c(n1)C(CC(O)C1CCC3(CC1)CC3)n1cncc1-2] | 53b |
| 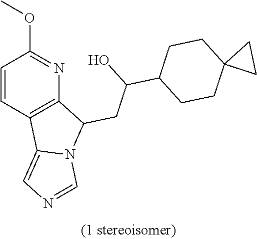 [SMILES: COc1ccc2c(n1)C(CC(O)C1CCC3(CC1)CC3)n1cncc1-2] | 53c |
| 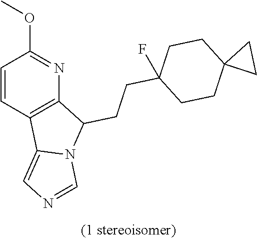 [SMILES: COc1ccc2c(n1)C(CCC1(F)CCC3(CC1)CC3)n1cncc1-2] | 54a |
| 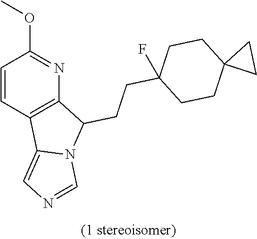 [SMILES: COc1ccc2c(n1)C(CCC1(F)CCC3(CC1)CC3)n1cncc1-2] | 54b |
| 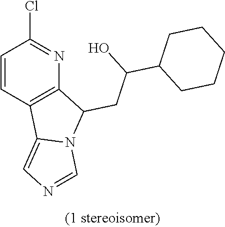 [SMILES: OC(CC1c2nc(Cl)ccc2-c2cncn21)C1CCCCC1] | 55a |
| 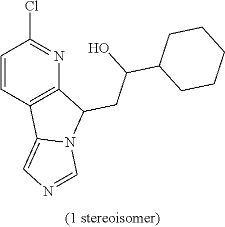 [SMILES: OC(CC1c2nc(Cl)ccc2-c2cncn21)C1CCCCC1] | 55b |
| 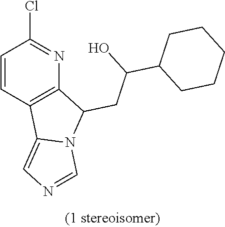 [SMILES: OC(CC1c2nc(Cl)ccc2-c2cncn21)C1CCCCC1] | 55c |
| 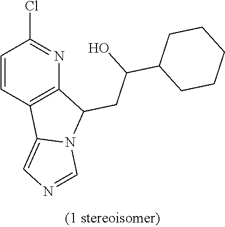 [SMILES: OC(CC1c2nc(Cl)ccc2-c2cncn21)C1CCCCC1] | 55d |
| 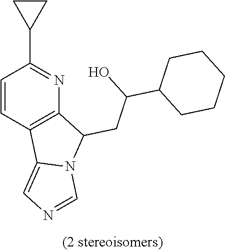 [SMILES: OC(CC1c2nc(C3CC3)ccc2-c2cncn21)C1CCCCC1] | 56a |
| 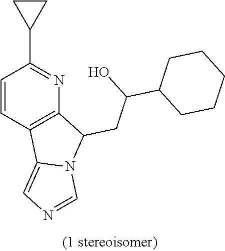 [SMILES: OC(CC1c2nc(C3CC3)ccc2-c2cncn21)C1CCCCC1] | 56b |
| 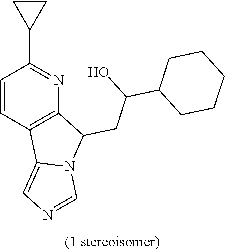 [SMILES: OC(CC1c2nc(C3CC3)ccc2-c2cncn21)C1CCCCC1] | 56c |
| 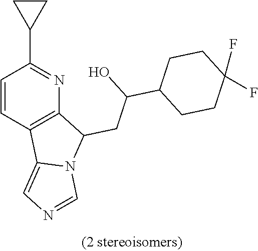 [SMILES: OC(CC1c2nc(C3CC3)ccc2-c2cncn21)C1CCC(F)(F)CC1] | 57a |
| 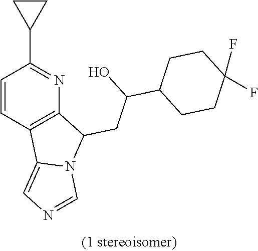 [SMILES: OC(CC1c2nc(C3CC3)ccc2-c2cncn21)C1CCC(F)(F)CC1] | 57b |
| 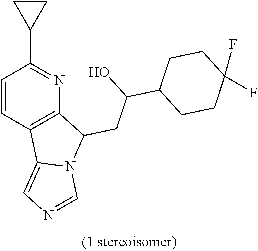 [SMILES: OC(CC1c2nc(C3CC3)ccc2-c2cncn21)C1CCC(F)(F)CC1] | 57c |
| 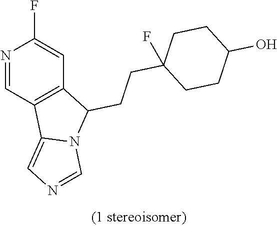 [SMILES: OC1CCC(F)(CCC2c3cc(F)ncc3-c3cncn32)CC1] | 58a |
| 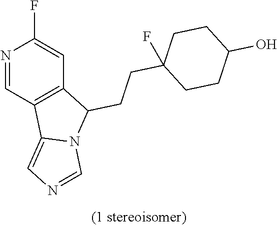 [SMILES: OC1CCC(F)(CCC2c3cc(F)ncc3-c3cncn32)CC1] | 58b |
| 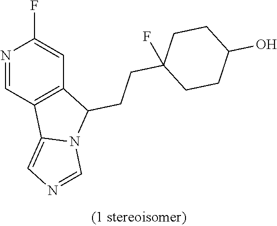 [SMILES: OC1CCC(F)(CCC2c3cc(F)ncc3-c3cncn32)CC1] | 58c |
| 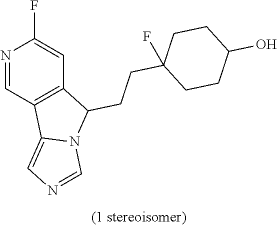 [SMILES: OC1CCC(F)(CCC2c3cc(F)ncc3-c3cncn32)CC1] | 58d |
| 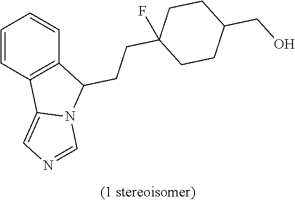 [SMILES: OCC1CCC(F)(CCC2c3ccccc3-c3cncn32)CC1] | 59a |
| 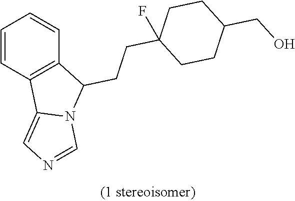 [SMILES: OCC1CCC(F)(CCC2c3ccccc3-c3cncn32)CC1] | 59b |
| 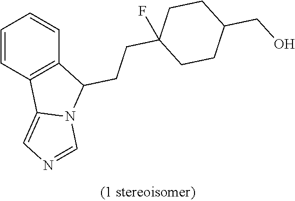 [SMILES: OCC1CCC(F)(CCC2c3ccccc3-c3cncn32)CC1] | 59c |
| 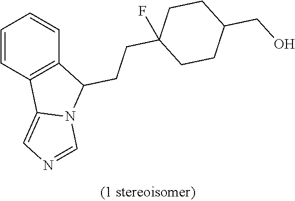 [SMILES: OCC1CCC(F)(CCC2c3ccccc3-c3cncn32)CC1] | 59d |
| 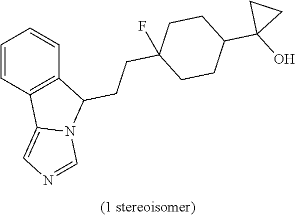 [SMILES: OC1(C2CCC(F)(CCC3c4ccccc4-c4cncn43)CC2)CC1] | 60a |
| 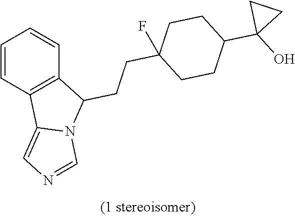 [SMILES: OC1(C2CCC(F)(CCC3c4ccccc4-c4cncn43)CC2)CC1] | 60b |
| 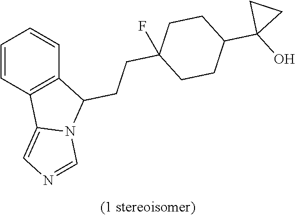 [SMILES: OC1(C2CCC(F)(CCC3c4ccccc4-c4cncn43)CC2)CC1] | 60c |
| 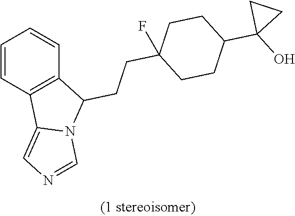 [SMILES: OC1(C2CCC(F)(CCC3c4ccccc4-c4cncn43)CC2)CC1] | 60d |
| 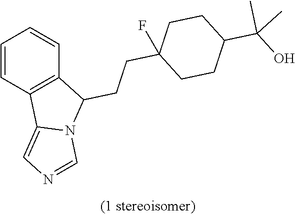 [SMILES: CC(C)(O)C1CCC(F)(CCC2c3ccccc3-c3cncn32)CC1] | 61a |
| 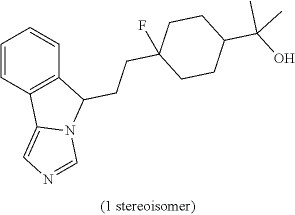 [SMILES: CC(C)(O)C1CCC(F)(CCC2c3ccccc3-c3cncn32)CC1] | 61b |
| 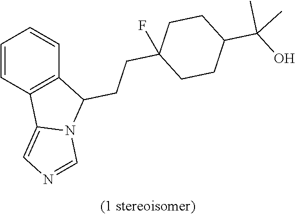 [SMILES: CC(C)(O)C1CCC(F)(CCC2c3ccccc3-c3cncn32)CC1] | 61c |
| 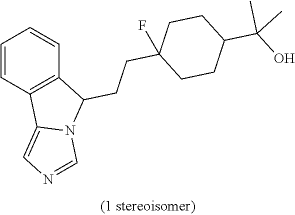 [SMILES: CC(C)(O)C1CCC(F)(CCC2c3ccccc3-c3cncn32)CC1] | 61d |
| 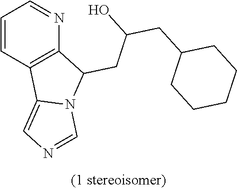 [SMILES: OC(CC1CCCCC1)CC1c2ncccc2-c2cncn21] | 62a |
| 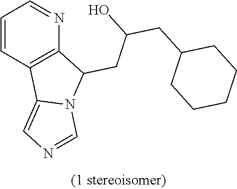 [SMILES: OC(CC1CCCCC1)CC1c2ncccc2-c2cncn21] | 62b |
| 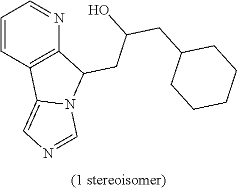 [SMILES: OC(CC1CCCCC1)CC1c2ncccc2-c2cncn21] | 62c |
| 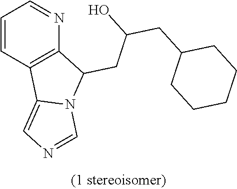 [SMILES: OC(CC1CCCCC1)CC1c2ncccc2-c2cncn21] | 62d |
| 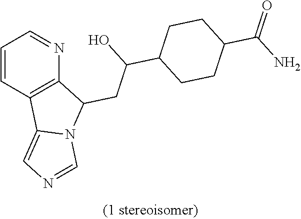 [SMILES: NC(=O)C1CCC(C(O)CC2c3ncccc3-c3cncn32)CC1] | 63a |
| 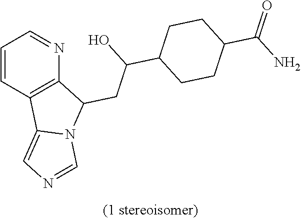 [SMILES: NC(=O)C1CCC(C(O)CC2c3ncccc3-c3cncn32)CC1] | 63b |
| 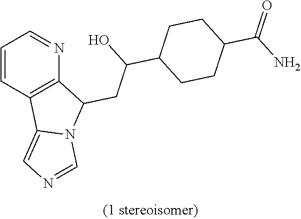 [SMILES: NC(=O)C1CCC(C(O)CC2c3ncccc3-c3cncn32)CC1] | 63c |
| 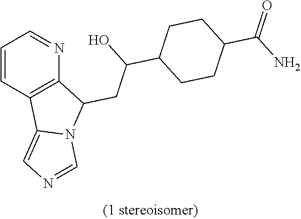 [SMILES: NC(=O)C1CCC(C(O)CC2c3ncccc3-c3cncn32)CC1] | 63d |
| 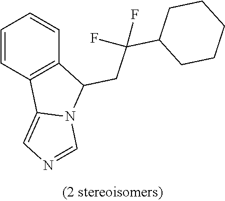 [SMILES: FC(F)(CC1c2ccccc2-c2cncn21)C1CCCCC1] | 64 |
| 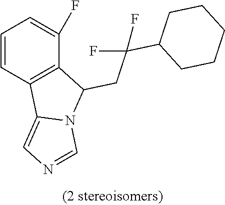 [SMILES: Fc1cccc2c1C(CC(F)(F)C1CCCCC1)n1cncc1-2] | 65 |
| 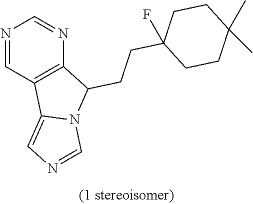 [SMILES: CC1(C)CCC(F)(CCC2c3ncncc3-c3cncn32)CC1] | 66a |
| 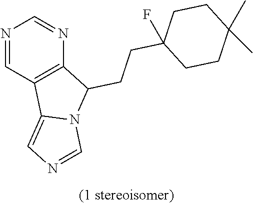 [SMILES: CC1(C)CCC(F)(CCC2c3ncncc3-c3cncn32)CC1] | 66b |
| 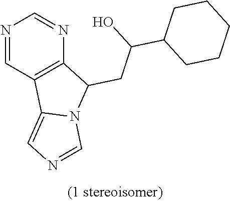 [SMILES: OC(CC1c2ncncc2-c2cncn21)C1CCCCC1] | 67a |
| 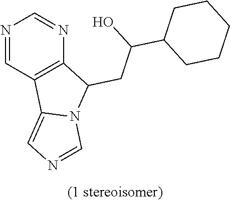 [SMILES: OC(CC1c2ncncc2-c2cncn21)C1CCCCC1] | 67b |
| 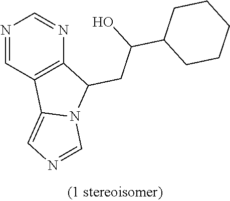 [SMILES: OC(CC1c2ncncc2-c2cncn21)C1CCCCC1] | 67c |
| 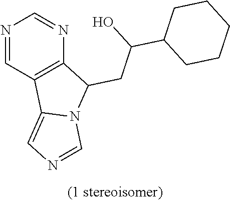 [SMILES: OC(CC1c2ncncc2-c2cncn21)C1CCCCC1] | 67d |
| 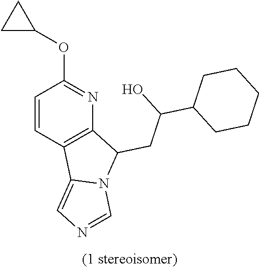 [SMILES: OC(CC1c2nc(OC3CC3)ccc2-c2cncn21)C1CCCCC1] | 68a |
| 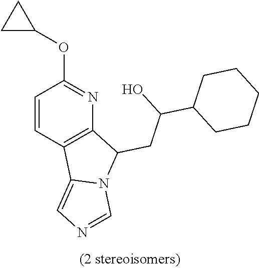 [SMILES: OC(CC1c2nc(OC3CC3)ccc2-c2cncn21)C1CCCCC1] | 68b |
| 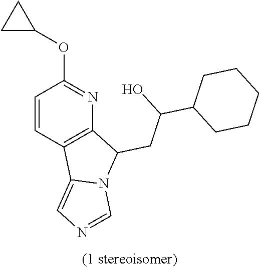 [SMILES: OC(CC1c2nc(OC3CC3)ccc2-c2cncn21)C1CCCCC1] | 68c |
| 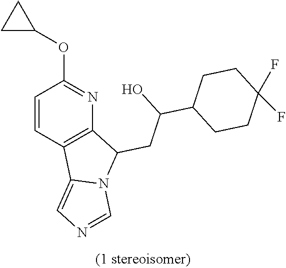 [SMILES: OC(CC1c2nc(OC3CC3)ccc2-c2cncn21)C1CCC(F)(F)CC1] | 69a |
| 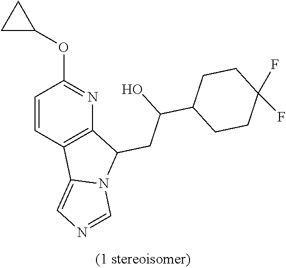 [SMILES: OC(CC1c2nc(OC3CC3)ccc2-c2cncn21)C1CCC(F)(F)CC1] | 69b |
| 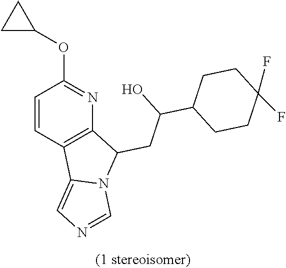 [SMILES: OC(CC1c2nc(OC3CC3)ccc2-c2cncn21)C1CCC(F)(F)CC1] | 69c |
| 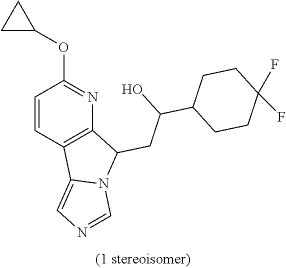 [SMILES: OC(CC1c2nc(OC3CC3)ccc2-c2cncn21)C1CCC(F)(F)CC1] | 69d |
| 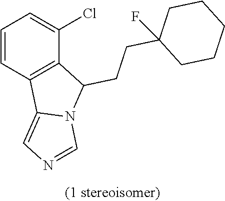 [SMILES: FC1(CCC2c3c(Cl)cccc3-c3cncn32)CCCCC1] | 70a |
| 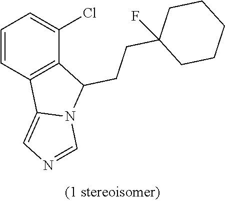 [SMILES: FC1(CCC2c3c(Cl)cccc3-c3cncn32)CCCCC1] | 70b |
| 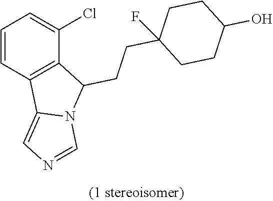 [SMILES: OC1CCC(F)(CCC2c3c(Cl)cccc3-c3cncn32)CC1] | 71a |
| 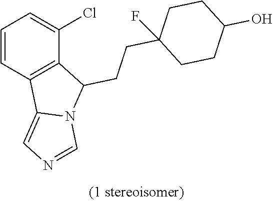 [SMILES: OC1CCC(F)(CCC2c3c(Cl)cccc3-c3cncn32)CC1] | 71b |
| 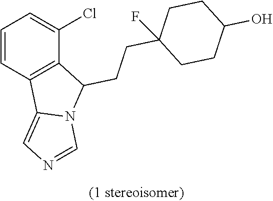 [SMILES: OC1CCC(F)(CCC2c3c(Cl)cccc3-c3cncn32)CC1] | 71c |
| 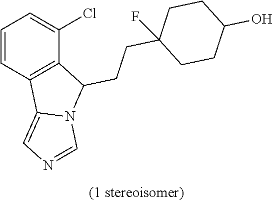 [SMILES: OC1CCC(F)(CCC2c3c(Cl)cccc3-c3cncn32)CC1] | 71d |
| 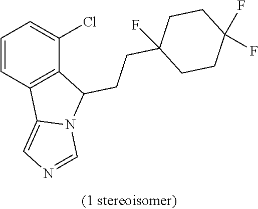 [SMILES: FC1(F)CCC(F)(CCC2c3c(Cl)cccc3-c3cncn32)CC1] | 72a |
| 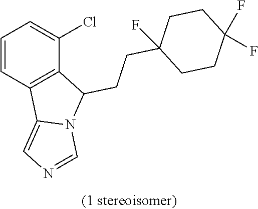 [SMILES: FC1(F)CCC(F)(CCC2c3c(Cl)cccc3-c3cncn32)CC1] | 72b |
| 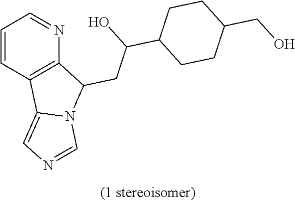 [SMILES: OCC1CCC(C(O)CC2c3ncccc3-c3cncn32)CC1] | 73a |
| 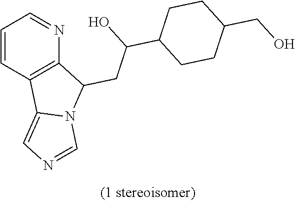 [SMILES: OCC1CCC(C(O)CC2c3ncccc3-c3cncn32)CC1] | 73b |
| 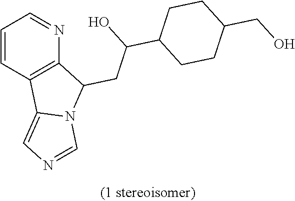 [SMILES: OCC1CCC(C(O)CC2c3ncccc3-c3cncn32)CC1] | 73c |
| 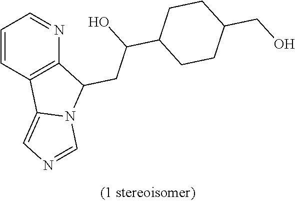 [SMILES: OCC1CCC(C(O)CC2c3ncccc3-c3cncn32)CC1] | 73d |
| 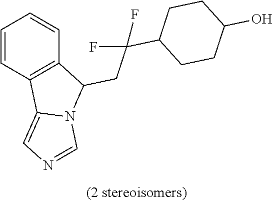 [SMILES: OC1CCC(C(F)(F)CC2c3ccccc3-c3cncn32)CC1] | 74a |
| 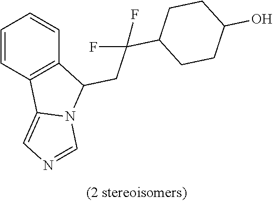 [SMILES: OC1CCC(C(F)(F)CC2c3ccccc3-c3cncn32)CC1] | 74b |
| 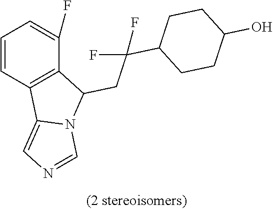 [SMILES: OC1CCC(C(F)(F)CC2c3c(F)cccc3-c3cncn32)CC1] | 75a |
| 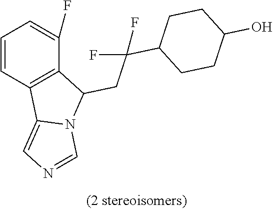 [SMILES: OC1CCC(C(F)(F)CC2c3c(F)cccc3-c3cncn32)CC1] | 75b |
| 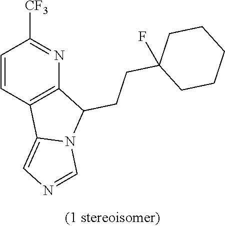 [SMILES: FC1(CCC2c3nc(C(F)(F)F)ccc3-c3cncn32)CCCCC1] | 76a |
| 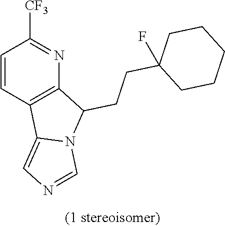 [SMILES: FC1(CCC2c3nc(C(F)(F)F)ccc3-c3cncn32)CCCCC1] | 76b |
| 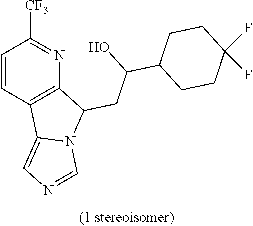 [SMILES: OC(CC1c2nc(C(F)(F)F)ccc2-c2cncn21)C1CCC(F)(F)CC1] | 77a |
| 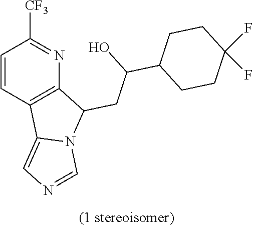 [SMILES: OC(CC1c2nc(C(F)(F)F)ccc2-c2cncn21)C1CCC(F)(F)CC1] | 77b |
| 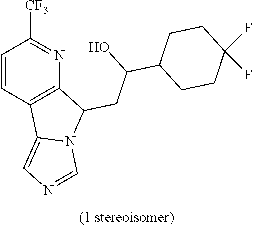 [SMILES: OC(CC1c2nc(C(F)(F)F)ccc2-c2cncn21)C1CCC(F)(F)CC1] | 77c |
| 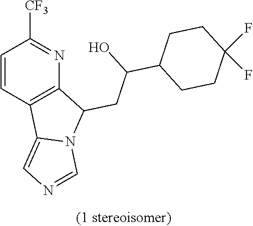 [SMILES: OC(CC1c2nc(C(F)(F)F)ccc2-c2cncn21)C1CCC(F)(F)CC1] | 77d |
| 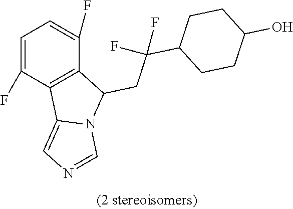 [SMILES: OC1CCC(C(F)(F)CC2c3c(F)ccc(F)c3-c3cncn32)CC1] | 78a |
| 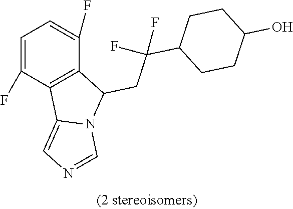 [SMILES: OC1CCC(C(F)(F)CC2c3c(F)ccc(F)c3-c3cncn32)CC1] | 78b |
| 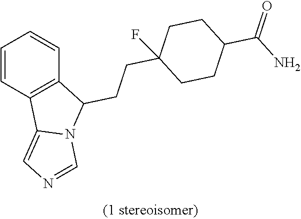 [SMILES: NC(=O)C1CCC(F)(CCC2c3ccccc3-c3cncn32)CC1] | 79a |
| 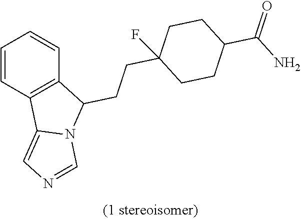 [SMILES: NC(=O)C1CCC(F)(CCC2c3ccccc3-c3cncn32)CC1] | 79b |
| 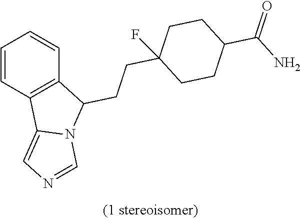 [SMILES: NC(=O)C1CCC(F)(CCC2c3ccccc3-c3cncn32)CC1] | 79c |
| 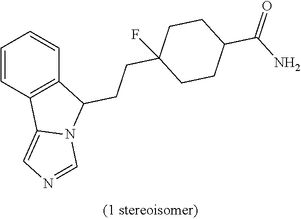 [SMILES: NC(=O)C1CCC(F)(CCC2c3ccccc3-c3cncn32)CC1] | 79d |
| 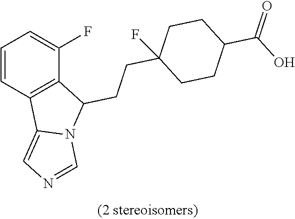 [SMILES: O=C(O)C1CCC(F)(CCC2c3c(F)cccc3-c3cncn32)CC1] | 80a |
| 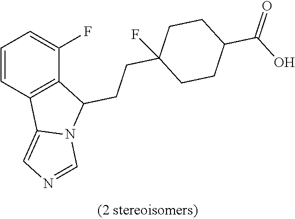 [SMILES: O=C(O)C1CCC(F)(CCC2c3c(F)cccc3-c3cncn32)CC1] | 80b |
| 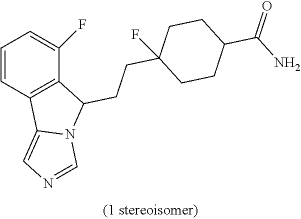 [SMILES: NC(=O)C1CCC(F)(CCC2c3c(F)cccc3-c3cncn32)CC1] | 81a |
| 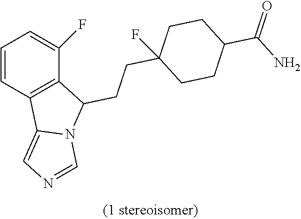 [SMILES: NC(=O)C1CCC(F)(CCC2c3c(F)cccc3-c3cncn32)CC1] | 81b |
| 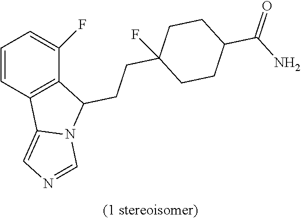 [SMILES: NC(=O)C1CCC(F)(CCC2c3c(F)cccc3-c3cncn32)CC1] | 81c |
| 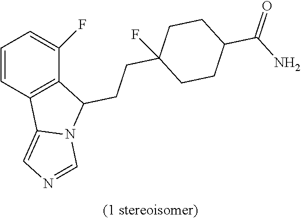 [SMILES: NC(=O)C1CCC(F)(CCC2c3c(F)cccc3-c3cncn32)CC1] | 81d |
| 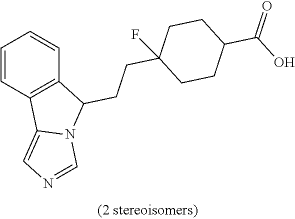 [SMILES: O=C(O)C1CCC(F)(CCC2c3ccccc3-c3cncn32)CC1] | 82a |
| 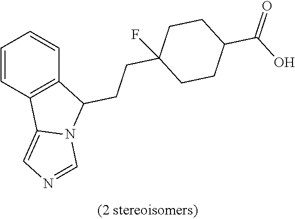 [SMILES: O=C(O)C1CCC(F)(CCC2c3ccccc3-c3cncn32)CC1] | 82b |
| 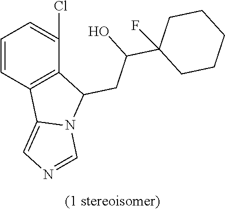 [SMILES: OC(CC1c2c(Cl)cccc2-c2cncn21)C1(F)CCCCC1] | 83a |
| 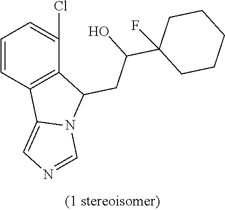 [SMILES: OC(CC1c2c(Cl)cccc2-c2cncn21)C1(F)CCCCC1] | 83b |
| 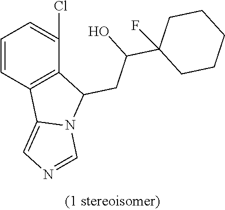 [SMILES: OC(CC1c2c(Cl)cccc2-c2cncn21)C1(F)CCCCC1] | 83c |
| 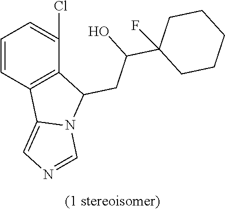 [SMILES: OC(CC1c2c(Cl)cccc2-c2cncn21)C1(F)CCCCC1] | 83d |
| 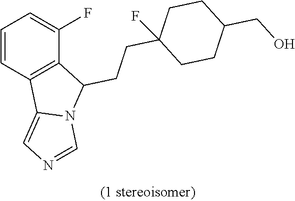 [SMILES: OCC1CCC(F)(CCC2c3c(F)cccc3-c3cncn32)CC1] | 84a |
| 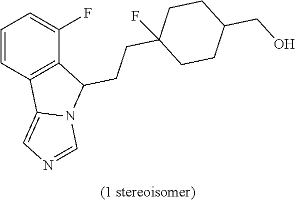 [SMILES: OCC1CCC(F)(CCC2c3c(F)cccc3-c3cncn32)CC1] | 84b |
| 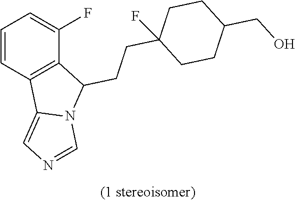 [SMILES: OCC1CCC(F)(CCC2c3c(F)cccc3-c3cncn32)CC1] | 84c |
| 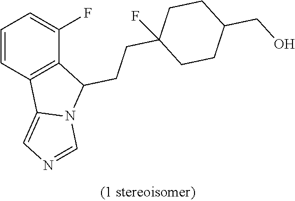 [SMILES: OCC1CCC(F)(CCC2c3c(F)cccc3-c3cncn32)CC1] | 84d |
| 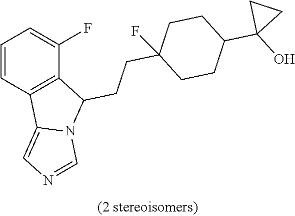 [SMILES: OC1(C2CCC(F)(CCC3c4c(F)cccc4-c4cncn43)CC2)CC1] | 85a |
| 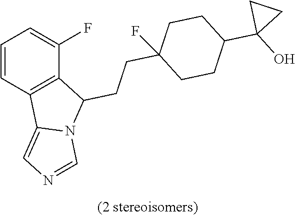 [SMILES: OC1(C2CCC(F)(CCC3c4c(F)cccc4-c4cncn43)CC2)CC1] | 85b |
| 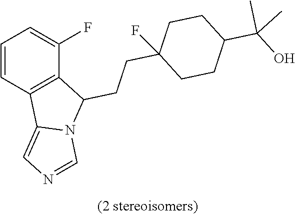 [SMILES: CC(C)(O)C1CCC(F)(CCC2c3c(F)cccc3-c3cncn32)CC1] | 86a |
| 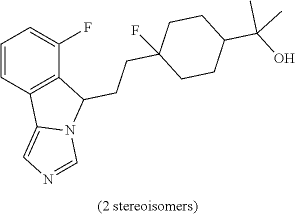 [SMILES: CC(C)(O)C1CCC(F)(CCC2c3c(F)cccc3-c3cncn32)CC1] | 86b |
| 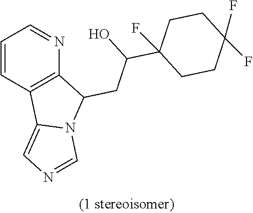 [SMILES: OC(CC1c2ncccc2-c2cncn21)C1(F)CCC(F)(F)CC1] | 87a |
| 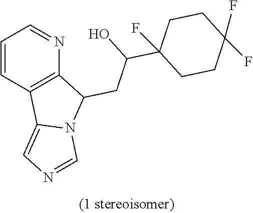 [SMILES: OC(CC1c2ncccc2-c2cncn21)C1(F)CCC(F)(F)CC1] | 87b |
| 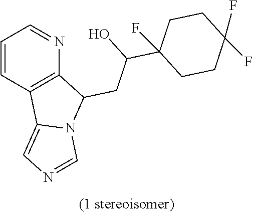 [SMILES: OC(CC1c2ncccc2-c2cncn21)C1(F)CCC(F)(F)CC1] | 87c |
| 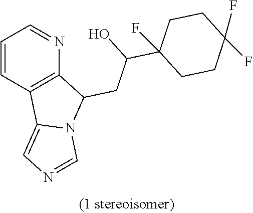 [SMILES: OC(CC1c2ncccc2-c2cncn21)C1(F)CCC(F)(F)CC1] | 87d |
| 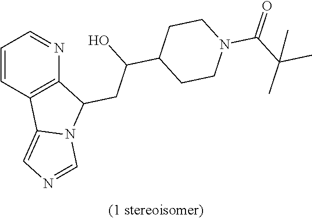 [SMILES: CC(C)(C)C(=O)N1CCC(C(O)CC2c3ncccc3-c3cncn32)CC1] | 88a |
| 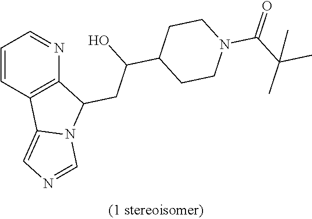 [SMILES: CC(C)(C)C(=O)N1CCC(C(O)CC2c3ncccc3-c3cncn32)CC1] | 88b |
| 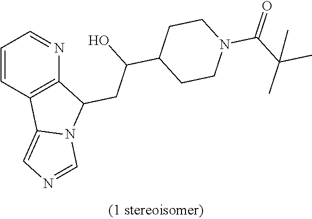 [SMILES: CC(C)(C)C(=O)N1CCC(C(O)CC2c3ncccc3-c3cncn32)CC1] | 88c |
| 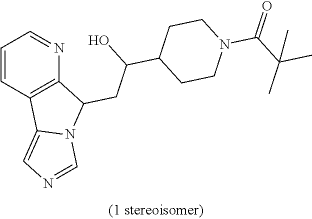 [SMILES: CC(C)(C)C(=O)N1CCC(C(O)CC2c3ncccc3-c3cncn32)CC1] | 88d |
| 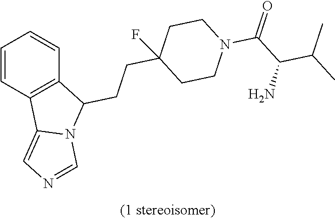 [SMILES: CC(C)[C@H](N)C(=O)N1CCC(F)(CCC2c3ccccc3-c3cncn32)CC1] | 89a |
| 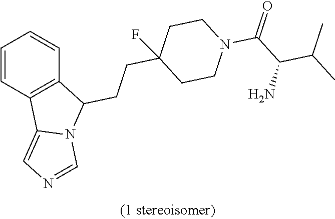 [SMILES: CC(C)[C@H](N)C(=O)N1CCC(F)(CCC2c3ccccc3-c3cncn32)CC1] | 89b |
| 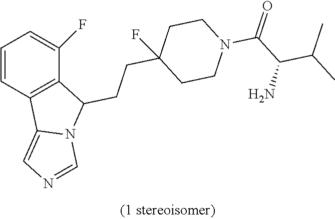 [SMILES: CC(C)[C@H](N)C(=O)N1CCC(F)(CCC2c3c(F)cccc3-c3cncn32)CC1] | 90a |
| 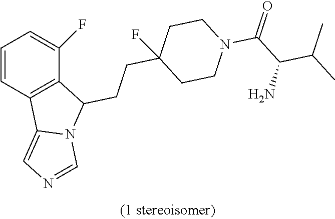 [SMILES: CC(C)[C@H](N)C(=O)N1CCC(F)(CCC2c3c(F)cccc3-c3cncn32)CC1] | 90b |
| 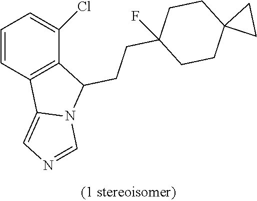 [SMILES: FC1(CCC2c3c(Cl)cccc3-c3cncn32)CCC2(CC1)CC2] | 91a |
| 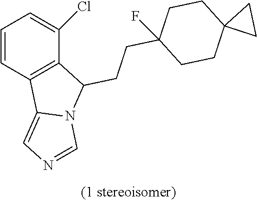 [SMILES: FC1(CCC2c3c(Cl)cccc3-c3cncn32)CCC2(CC1)CC2] | 91b |
| 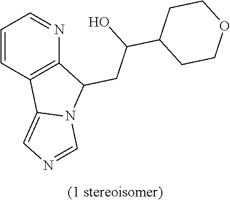 [SMILES: OC(CC1c2ncccc2-c2cncn21)C1CCOCC1] | 92a |
| 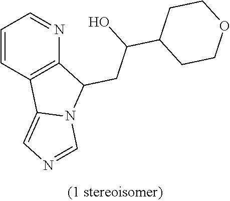 [SMILES: OC(CC1c2ncccc2-c2cncn21)C1CCOCC1] | 92b |
| 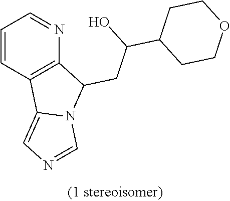 [SMILES: OC(CC1c2ncccc2-c2cncn21)C1CCOCC1] | 92c |
| 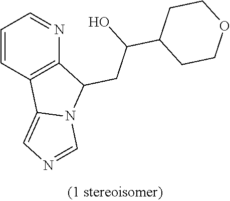 [SMILES: OC(CC1c2ncccc2-c2cncn21)C1CCOCC1] | 92d |
| 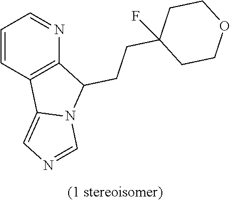 [SMILES: FC1(CCC2c3ncccc3-c3cncn32)CCOCC1] | 93a |
| 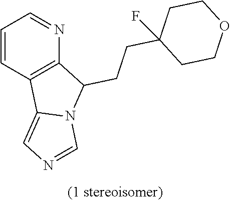 [SMILES: FC1(CCC2c3ncccc3-c3cncn32)CCOCC1] | 93b |
| 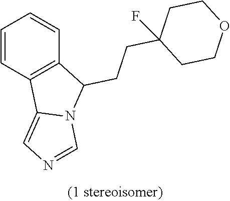 [SMILES: FC1(CCC2c3ccccc3-c3cncn32)CCOCC1] | 94a |
| 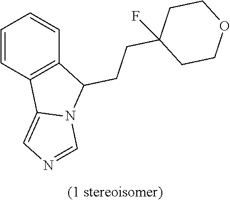 [SMILES: FC1(CCC2c3ccccc3-c3cncn32)CCOCC1] | 94b |
| 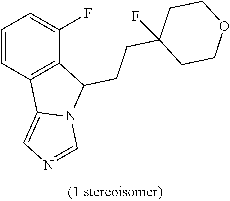 [SMILES: Fc1cccc2c1C(CCC1(F)CCOCC1)n1cncc1-2] | 95a |
| 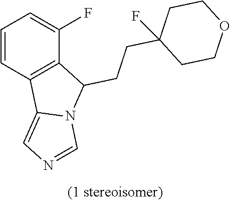 [SMILES: Fc1cccc2c1C(CCC1(F)CCOCC1)n1cncc1-2] | 95b |
| 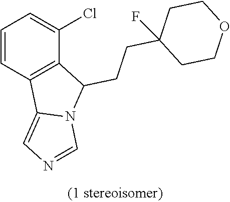 [SMILES: FC1(CCC2c3c(Cl)cccc3-c3cncn32)CCOCC1] | 96a |
| 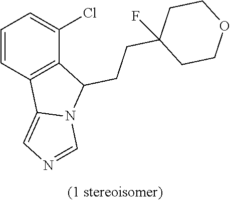 [SMILES: FC1(CCC2c3c(Cl)cccc3-c3cncn32)CCOCC1] | 96b |
| 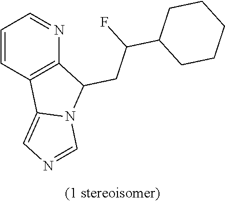 [SMILES: FC(CC1c2ncccc2-c2cncn21)C1CCCCC1] | 97a |
| 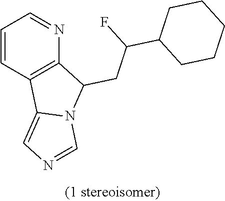 [SMILES: FC(CC1c2ncccc2-c2cncn21)C1CCCCC1] | 97b |
| 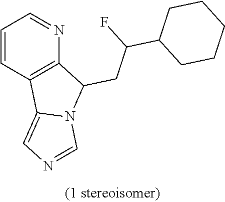 [SMILES: FC(CC1c2ncccc2-c2cncn21)C1CCCCC1] | 97c |
| 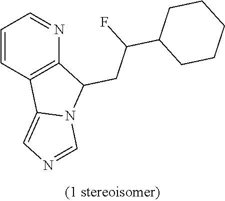 [SMILES: FC(CC1c2ncccc2-c2cncn21)C1CCCCC1] | 97d |
| 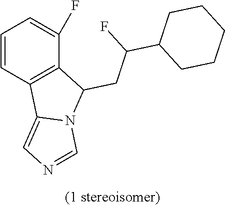 [SMILES: Fc1cccc2c1C(CC(F)C1CCCCC1)n1cncc1-2] | 98a |
| 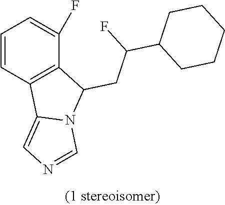 [SMILES: Fc1cccc2c1C(CC(F)C1CCCCC1)n1cncc1-2] | 98b |
| 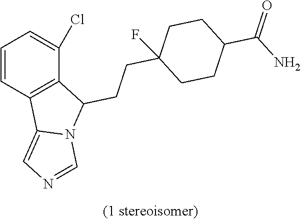 [SMILES: NC(=O)C1CCC(F)(CCC2c3c(Cl)cccc3-c3cncn32)CC1] | 99a |
| 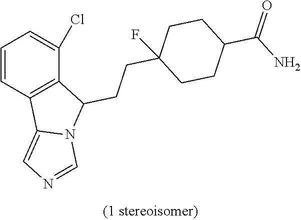 [SMILES: NC(=O)C1CCC(F)(CCC2c3c(Cl)cccc3-c3cncn32)CC1] | 99b |
| 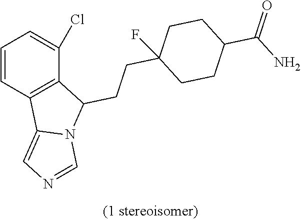 [SMILES: NC(=O)C1CCC(F)(CCC2c3c(Cl)cccc3-c3cncn32)CC1] | 99c |
| 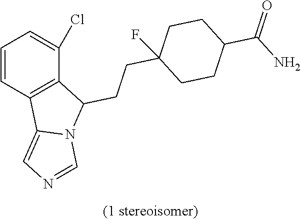 [SMILES: NC(=O)C1CCC(F)(CCC2c3c(Cl)cccc3-c3cncn32)CC1] | 99d |
| 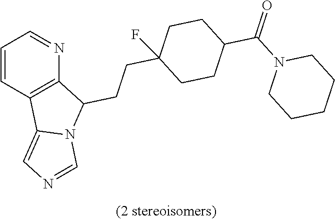 [SMILES: O=C(C1CCC(F)(CCC2c3ncccc3-c3cncn32)CC1)N1CCCCC1] | 100a |
| 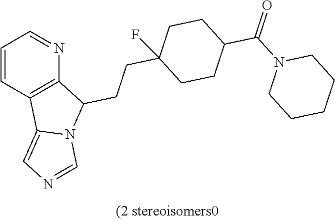 [SMILES: O=C(C1CCC(F)(CCC2c3ncccc3-c3cncn32)CC1)N1CCCCC1] | 100b |
| 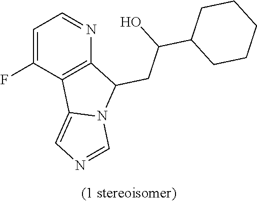 [SMILES: OC(CC1c2nccc(F)c2-c2cncn21)C1CCCCC1] | 101a |
| 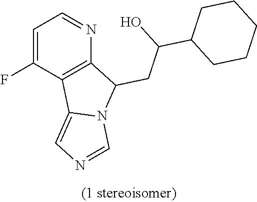 [SMILES: OC(CC1c2nccc(F)c2-c2cncn21)C1CCCCC1] | 101b |
| 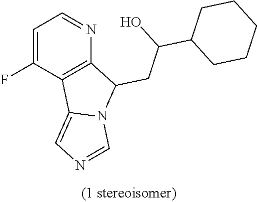 [SMILES: OC(CC1c2nccc(F)c2-c2cncn21)C1CCCCC1] | 101c |
| 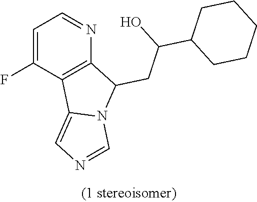 [SMILES: OC(CC1c2nccc(F)c2-c2cncn21)C1CCCCC1] | 101d |
| 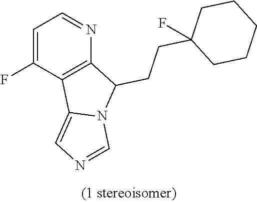 [SMILES: Fc1ccnc2c1-c1cncn1C2CCC1(F)CCCCC1] | 102a |
| 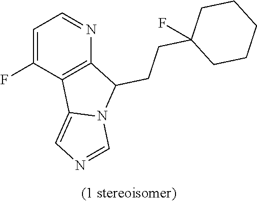 [SMILES: Fc1ccnc2c1-c1cncn1C2CCC1(F)CCCCC1] | 102b |
| 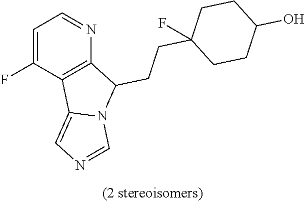 [SMILES: OC1CCC(F)(CCC2c3nccc(F)c3-c3cncn32)CC1] | 103a |
| 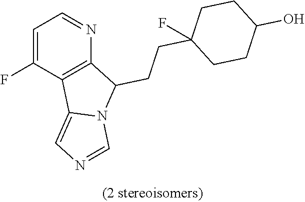 [SMILES: OC1CCC(F)(CCC2c3nccc(F)c3-c3cncn32)CC1] | 103b |
| 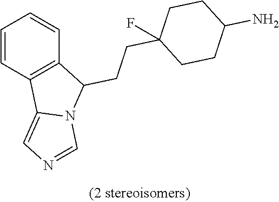 [SMILES: NC1CCC(F)(CCC2c3ccccc3-c3cncn32)CC1] | 104a |
| 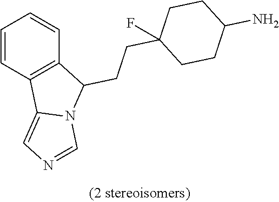 [SMILES: NC1CCC(F)(CCC2c3ccccc3-c3cncn32)CC1] | 104b |
| 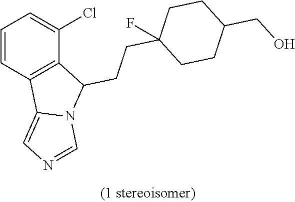 [SMILES: OCC1CCC(F)(CCC2c3c(Cl)cccc3-c3cncn32)CC1] | 105a |
| 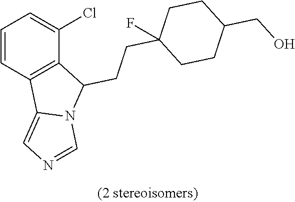 [SMILES: OCC1CCC(F)(CCC2c3c(Cl)cccc3-c3cncn32)CC1] | 105b |
| 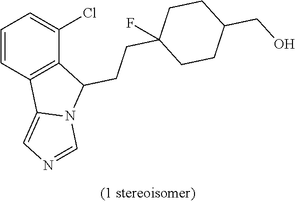 [SMILES: OCC1CCC(F)(CCC2c3c(Cl)cccc3-c3cncn32)CC1] | 105c |
| 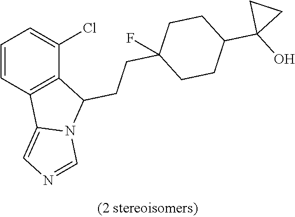 [SMILES: OC1(C2CCC(F)(CCC3c4c(Cl)cccc4-c4cncn43)CC2)CC1] | 106a |
| 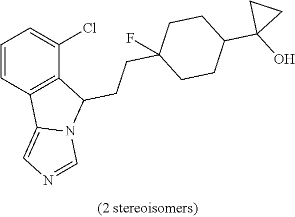 [SMILES: OC1(C2CCC(F)(CCC3c4c(Cl)cccc4-c4cncn43)CC2)CC1] | 106b |
| 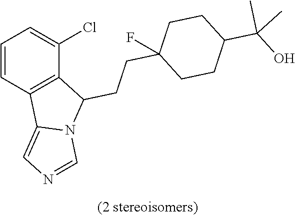 [SMILES: CC(C)(O)C1CCC(F)(CCC2c3c(Cl)cccc3-c3cncn32)CC1] | 107a |
| 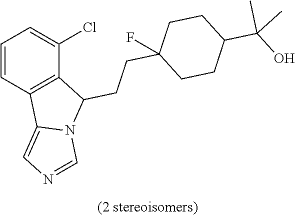 [SMILES: CC(C)(O)C1CCC(F)(CCC2c3c(Cl)cccc3-c3cncn32)CC1] | 107b |
| 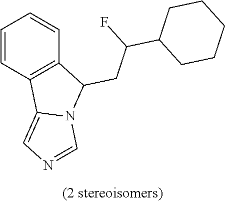 [SMILES: FC(CC1c2ccccc2-c2cncn21)C1CCCCC1] | 108a |
| 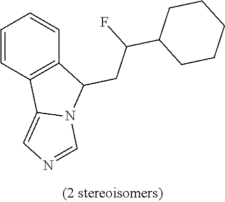 [SMILES: FC(CC1c2ccccc2-c2cncn21)C1CCCCC1] | 108b |
| 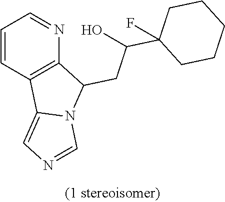 [SMILES: OC(CC1c2ncccc2-c2cncn21)C1(F)CCCCC1] | 109a |
| 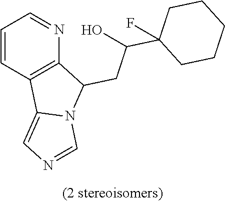 [SMILES: OC(CC1c2ncccc2-c2cncn21)C1(F)CCCCC1] | 109b |
| 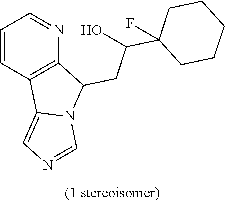 [SMILES: OC(CC1c2ncccc2-c2cncn21)C1(F)CCCCC1] | 109c |
| 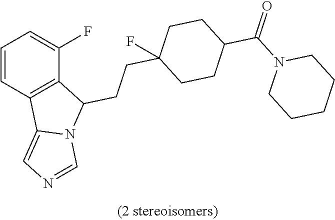 [SMILES: O=C(C1CCC(F)(CCC2c3c(F)cccc3-c3cncn32)CC1)N1CCCCC1] | 110a |
| 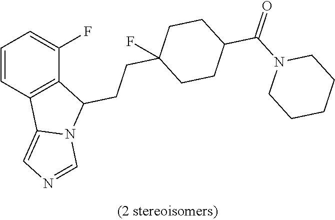 [SMILES: O=C(C1CCC(F)(CCC2c3c(F)cccc3-c3cncn32)CC1)N1CCCCC1] | 100b |
| 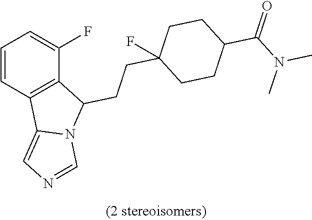 [SMILES: CN(C)C(=O)C1CCC(F)(CCC2c3c(F)cccc3-c3cncn32)CC1] | 111a |
| 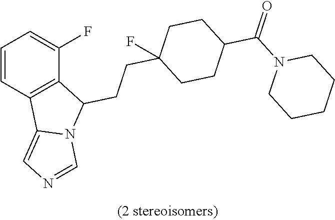 [SMILES: O=C(C1CCC(F)(CCC2c3c(F)cccc3-c3cncn32)CC1)N1CCCCC1] | 111b |
| 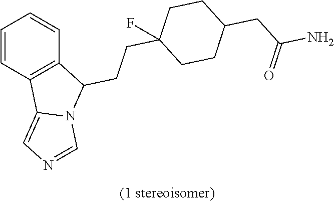 [SMILES: NC(=O)CC1CCC(F)(CCC2c3ccccc3-c3cncn32)CC1] | 112a |
| 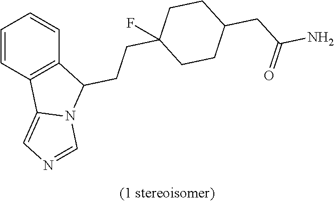 [SMILES: NC(=O)CC1CCC(F)(CCC2c3ccccc3-c3cncn32)CC1] | 112b |
| 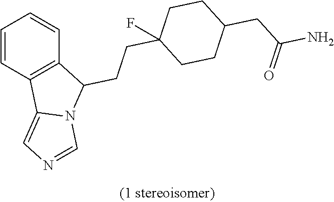 [SMILES: NC(=O)CC1CCC(F)(CCC2c3ccccc3-c3cncn32)CC1] | 112c |
| 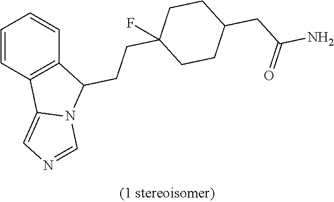 [SMILES: NC(=O)CC1CCC(F)(CCC2c3ccccc3-c3cncn32)CC1] | 112d |
| 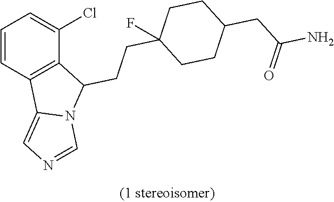 [SMILES: NC(=O)CC1CCC(F)(CCC2c3c(Cl)cccc3-c3cncn32)CC1] | 113a |
| 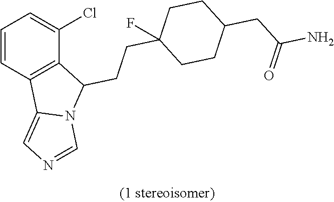 [SMILES: NC(=O)CC1CCC(F)(CCC2c3c(Cl)cccc3-c3cncn32)CC1] | 113b |
| 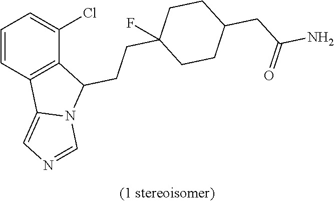 [SMILES: NC(=O)CC1CCC(F)(CCC2c3c(Cl)cccc3-c3cncn32)CC1] | 113c |
| 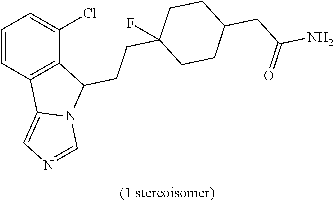 [SMILES: NC(=O)CC1CCC(F)(CCC2c3c(Cl)cccc3-c3cncn32)CC1] | 113d |
| 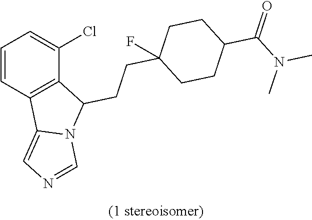 [SMILES: CN(C)C(=O)C1CCC(F)(CCC2c3c(Cl)cccc3-c3cncn32)CC1] | 114a |
| 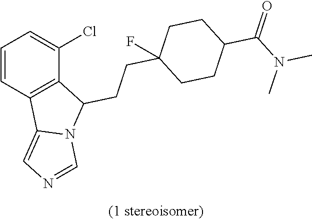 [SMILES: CN(C)C(=O)C1CCC(F)(CCC2c3c(Cl)cccc3-c3cncn32)CC1] | 114b |
| 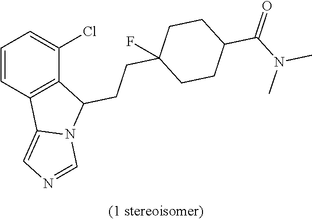 [SMILES: CN(C)C(=O)C1CCC(F)(CCC2c3c(Cl)cccc3-c3cncn32)CC1] | 114c |
| 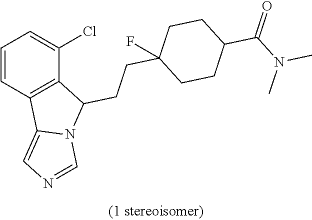 [SMILES: CN(C)C(=O)C1CCC(F)(CCC2c3c(Cl)cccc3-c3cncn32)CC1] | 114d |
| 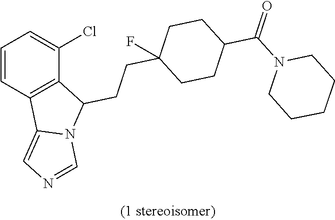 [SMILES: O=C(C1CCC(F)(CCC2c3c(Cl)cccc3-c3cncn32)CC1)N1CCCCC1] | 115a |
| 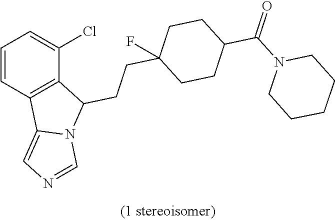 [SMILES: O=C(C1CCC(F)(CCC2c3c(Cl)cccc3-c3cncn32)CC1)N1CCCCC1] | 115b |
| 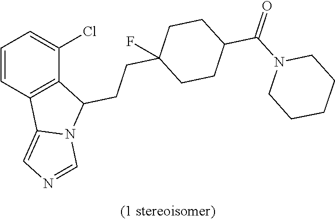 [SMILES: O=C(C1CCC(F)(CCC2c3c(Cl)cccc3-c3cncn32)CC1)N1CCCCC1] | 115c |
| 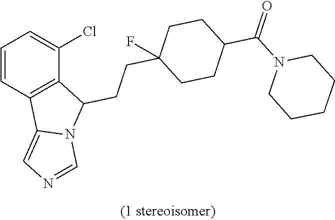 [SMILES: O=C(C1CCC(F)(CCC2c3c(Cl)cccc3-c3cncn32)CC1)N1CCCCC1] | 115d |
| 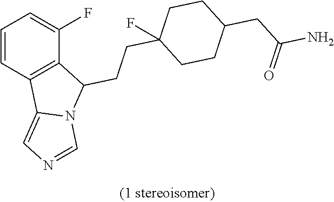 [SMILES: NC(=O)CC1CCC(F)(CCC2c3c(F)cccc3-c3cncn32)CC1] | 116a |
| 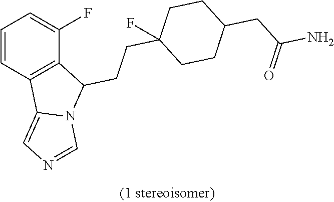 [SMILES: NC(=O)CC1CCC(F)(CCC2c3c(F)cccc3-c3cncn32)CC1] | 116b |
| 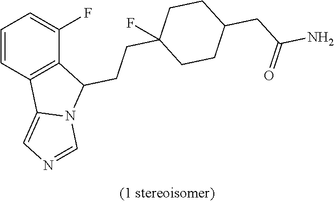 [SMILES: NC(=O)CC1CCC(F)(CCC2c3c(F)cccc3-c3cncn32)CC1] | 116c |
| 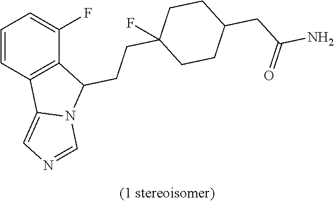 [SMILES: NC(=O)CC1CCC(F)(CCC2c3c(F)cccc3-c3cncn32)CC1] | 116d |
| 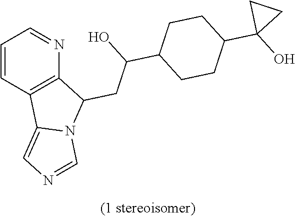 [SMILES: OC(CC1c2ncccc2-c2cncn21)C1CCC(C2(O)CC2)CC1] | 117a |
| 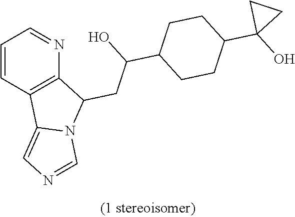 [SMILES: OC(CC1c2ncccc2-c2cncn21)C1CCC(C2(O)CC2)CC1] | 117b |
| 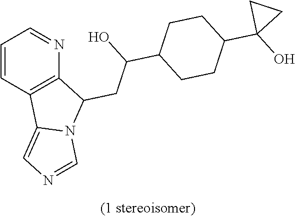 [SMILES: OC(CC1c2ncccc2-c2cncn21)C1CCC(C2(O)CC2)CC1] | 117c |
| 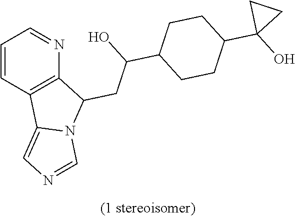 [SMILES: OC(CC1c2ncccc2-c2cncn21)C1CCC(C2(O)CC2)CC1] | 117d |
| 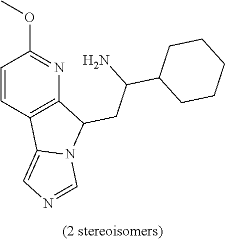 [SMILES: COc1ccc2c(n1)C(CC(N)C1CCCCC1)n1cncc1-2] | 118a |
| 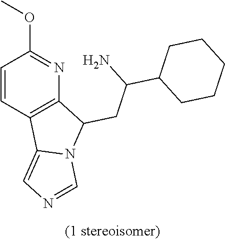 [SMILES: COc1ccc2c(n1)C(CC(N)C1CCCCC1)n1cncc1-2] | 118b |
| 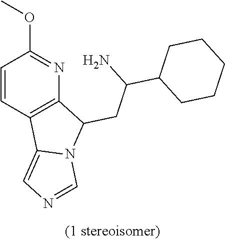 [SMILES: COc1ccc2c(n1)C(CC(N)C1CCCCC1)n1cncc1-2] | 118c |
| 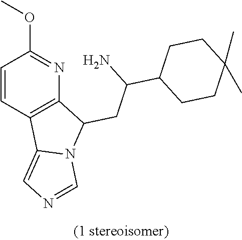 [SMILES: COc1ccc2c(n1)C(CC(N)C1CCC(C)(C)CC1)n1cncc1-2] | 119a |
| 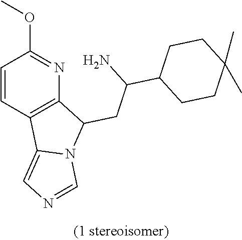 [SMILES: COc1ccc2c(n1)C(CC(N)C1CCC(C)(C)CC1)n1cncc1-2] | 119b |
| 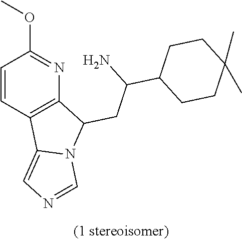 [SMILES: COc1ccc2c(n1)C(CC(N)C1CCC(C)(C)CC1)n1cncc1-2] | 119c |
| 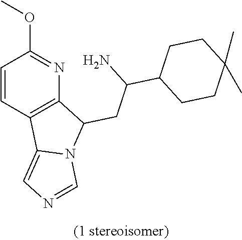 [SMILES: COc1ccc2c(n1)C(CC(N)C1CCC(C)(C)CC1)n1cncc1-2] | 119d |
| 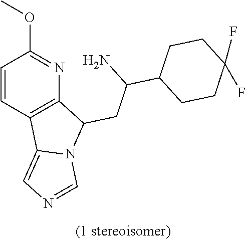 [SMILES: COc1ccc2c(n1)C(CC(N)C1CCC(F)(F)CC1)n1cncc1-2] | 120a |
| 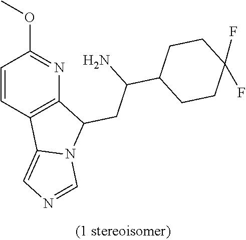 [SMILES: COc1ccc2c(n1)C(CC(N)C1CCC(F)(F)CC1)n1cncc1-2] | 120b |
| 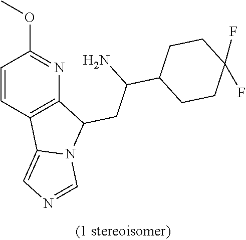 [SMILES: COc1ccc2c(n1)C(CC(N)C1CCC(F)(F)CC1)n1cncc1-2] | 120c |
| 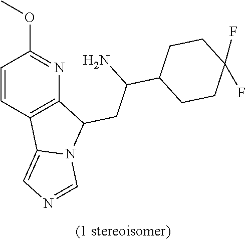 [SMILES: COc1ccc2c(n1)C(CC(N)C1CCC(F)(F)CC1)n1cncc1-2] | 120d |
| 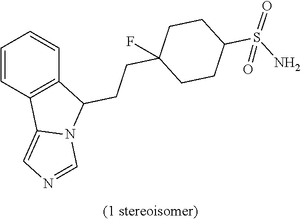 [SMILES: NS(=O)(=O)C1CCC(F)(CCC2c3ccccc3-c3cncn32)CC1] | 121a |
| 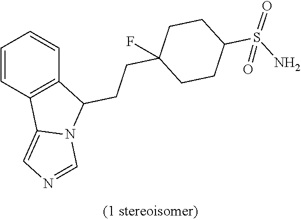 [SMILES: NS(=O)(=O)C1CCC(F)(CCC2c3ccccc3-c3cncn32)CC1] | 121b |
| 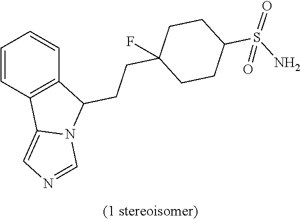 [SMILES: NS(=O)(=O)C1CCC(F)(CCC2c3ccccc3-c3cncn32)CC1] | 121c |
| 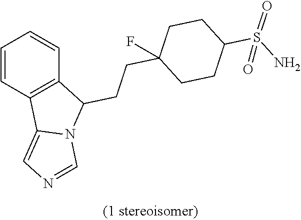 [SMILES: NS(=O)(=O)C1CCC(F)(CCC2c3ccccc3-c3cncn32)CC1] | 121d |

In some embodiments, the present invention provides a compound selected from those depicted above, or a pharmaceutically acceptable salt thereof.

Various structural depictions may show a heteroatom without an attached group, radical, charge, or counterion. Those of ordinary skill in the art are aware that such depictions are meant to indicate that the heteroatom is attached to hydrogen (e.g.,

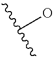 [SMILES: *O]

is understood to be

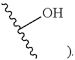 [SMILES: *O]

In certain embodiments, the compounds of the invention were synthesized in accordance with Schemes below. More specific examples of compounds made utilizing the Schemes are provided in the Examples below.

In some aspects, the IDO1 inhibitor is 4-fluoro-4-[2-[5H-imidazo[4,3-a]isoindol-5-yl]ethyl]cyclohexane-1-sulfonamide, or a pharmaceutically acceptable salt thereof, having the structure of Compound 1:

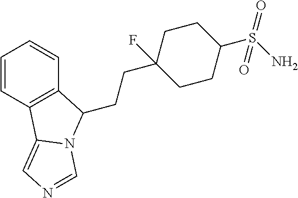 [SMILES: NS(=O)(=O)C1CCC(F)(CCC2c3ccccc3-c3cncn32)CC1]

or a pharmaceutically acceptable salt thereof.

In some aspects, the IDO1 inhibitor is c-4-fluoro-t-4-[(S)-2-(5H-imidazo[5,1-a]isoindol-5-yl)-ethyl]-cyclohexane-r-1-sulfonic acid amide, or a pharmaceutically acceptable salt thereof, having the structure of Compound 2:

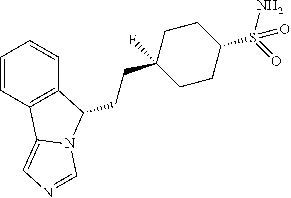 [SMILES: NS(=O)(=O)[C@H]1CC[C@](F)(CC[C@H]2c3ccccc3-c3cncn32)CC1]

or a pharmaceutically acceptable salt thereof.

Compound 1 or 2 is described in detail in United States patent application US 2016/075711, published on Mar. 17, 2016 (referred to herein as “the '711 publication”), the entirety of which is hereby incorporated herein by reference. Compound 1 is designated as compound 121 in Table 1 of the '711 publication. Compound 2 is designated as compound 121a, b, c, d in Example 121 of the '711 publication or Example 1 of the present specification. Compound 1 or 2 is active in a variety of assays and therapeutic models demonstrating inhibition of IDO1 (see, e.g., Example 122 of the '711 publication). Accordingly, Compound 1 or 2, or a pharmaceutically acceptable salt thereof, is useful for treating one or more disorders associated with activity of IDO1, as described in detail herein.

The IDO1 inhibitor of the invention includes any non-toxic salt, ester, salt of an ester or other derivative of a compound of this invention that, upon administration to a recipient, is capable of providing, either directly or indirectly, a compound of this invention or an inhibitory active metabolite or residue thereof. It is understood that although the methods described herein may refer to formulations, doses and dosing regimens/schedules of Compound 1 or 2, such formulations, doses and/or dosing regimens/schedules are equally applicable to any pharmaceutically acceptable salt of Compound 1 or 2. Accordingly, in some embodiments, a dose or dosing regimen for a pharmaceutically acceptable salt of Compound 1 or 2, or a pharmaceutically acceptable salt thereof, is selected from any of the doses or dosing regimens for Compound 1 or 2 as described herein.

A pharmaceutically acceptable salt may involve the inclusion of another molecule, such as an acetate ion, a succinate ion or other counter ion. The counter ion may be any organic or inorganic moiety that stabilizes the charge on the parent compound. Furthermore, a pharmaceutically acceptable salt may have more than one charged atom in its structure. Instances where multiple charged atoms are part of the pharmaceutically acceptable salt can have multiple counter ions. Hence, a pharmaceutically acceptable salt can have one or more charged atoms and/or one or more counter ion. If the compound of the invention is a base, the desired pharmaceutically acceptable salt may be prepared by any suitable method available in the art, for example, treatment of the free base with an inorganic acid, such as hydrochloric acid, hydrobromic acid, sulfuric acid, nitric acid, methanesulfonic acid, phosphoric acid and the like, or with an organic acid, such as acetic acid, maleic acid, succinic acid, mandelic acid, fumaric acid, malonic acid, pyruvic acid, oxalic acid, glycolic acid, salicylic acid, a pyranosidyl acid, such as glucuronic acid or galacturonic acid, an alpha hydroxy acid, such as citric acid or tartaric acid, an amino acid, such as aspartic acid or glutamic acid, an aromatic acid, such as benzoic acid or cinnamic acid, a sulfonic acid, such as p-toluenesulfonic acid or ethanesulfonic acid, or the like. If the compound of the invention is an acid, the desired pharmaceutically acceptable salt may be prepared by any suitable method, for example, treatment of the free acid with an inorganic or organic base, such as an amine (primary, secondary or tertiary), an alkali metal hydroxide or alkaline earth metal hydroxide, or the like. Illustrative examples of suitable salts include, but are not limited to, organic salts derived from amino acids, such as glycine and arginine, ammonia, primary, secondary, and tertiary amines, and cyclic amines, such as piperidine, morpholine and piperazine, and inorganic salts derived from sodium, calcium, potassium, magnesium, manganese, iron, copper, zinc, aluminum and lithium.

In one embodiment, the therapeutic combination of the invention is used in the treatment of a human subject. In one embodiment, the anti-PD-L1 antibody targets PD-L1 which is human PD-L1. The main expected benefit in the treatment with the therapeutic combination is a gain in risk/benefit ratio with said antibody, particularly avelumab, for these human patients.

In one embodiment, the cancer is identified as a PD-L1-positive and/or IDO1-positive cancerous disease. Pharmacodynamic analyses show that tumor expression of PD-L1 and/or IDO1 might be predictive of treatment efficacy. According to the invention, the cancer is preferably considered to be PD-L1 positive if between at least 0.1% and at least 10% of the cells of the cancer have PD-L1 present at their cell surface, more preferably between at least 0.5% and 5%, most preferably at least 1%. In one embodiment, the PD-L1 expression is determined by immunohistochemistry (IHC). Immunohistochemistry with anti-PD-L1 primary antibodies can be performed on serial cuts of formalin fixed and paraffin embedded specimens from patients treated with a PD-1 antagonist, such as avelumab, and an IDO1 inhibitor.

According to the invention, the cancer is preferably considered to be an IDO1-positive or IDO1-expressing cancer if its IDO1 level exceeds an IDO1 level predetermined prior to administering to a subject the anti-PD-L1 antibody and/or the IDO1 inhibitor. The IDO1-positive cancer shows an IDO1 expression that exceeds an IDO1 level predetermined prior to administering to the subject the anti-PD-L1 antibody and/or the IDO1 inhibitor. The addition of an IDO1 inhibitor is particularly beneficial in subjects that show an increase in IDO1 expression compared to a baseline, i.e., a predetermined value, either due to an innate tumor resistance mechanism or through an expressed tumor resistant mechanism, e.g., as a consequence of the treatment with an immunotherapy agent, such as avelumab. In one embodiment, the cancer is innately resistant to cancer therapy, preferably immunotherapy, more preferably checkpoint inhibitor treatment, or the cancer was resistant or became resistant to prior cancer therapy, preferably immunotherapy, more preferably checkpoint inhibitor treatment, in each case either in part or completely. Upregulation of IDO1/TDO2 in tumors induces a dominant immune resistant tumor environment and escape pathway to checkpoint inhibitor treatment. In one aspect of the invention, the cancer is an IDO1-positive cancer (having upregulated IDO1 expression) that induces or provides an escape pathway to or escapes checkpoint inhibitor treatment. Such an IDO1-positive cancer suppresses checkpoint inhibitor activity, preferably an IDO1-high expressing cancer that is characterized by the presence of high levels of IDO1, compared to a predetermined value. Inhibition of this pathway, in combination with checkpoint inhibitors, restores and enhances antitumor responses. IDO1 is therefore a useful biomarker for the selection of patients that receive and respond to IDO1/anti-PD-L1 combination therapy. IDO1 efficiently induces immune cell stimulation and rescues the suppressed activity of anti-PD-L1 antibodies when used in combination therapy in patients with IDO1-high expressing cancers.

The invention provides for the treatment of diseases, disorders, and conditions characterized by excessive or abnormal cell proliferation. Such diseases include a proliferative or hyperproliferative disease. Examples of proliferative and hyperproliferative diseases include cancer and myeloproliferative disorders.

In certain embodiments, the term “cancer” includes, but is not limited to the following cancers. Oral: head and neck, including buccal cavity, lip, tongue, mouth, pharynx; Cardiac: sarcoma (angiosarcoma, fibrosarcoma, rhabdomyosarcoma, liposarcoma), myxoma, rhabdomyoma, fibroma, lipoma and teratoma; Lung: Non-small cell lung carcinoma including adenocarcinoma (acinar, bronchioloalveolar carcinoma [nonmucinous, mucinous, mixed], papillary, solid adenocarcionoma, clear cell, mucinous [colloid] adenocarcinoma, mucinous cystadenocarcinoma, signet ring, well-differentiated fetal), bronchioalveolar, squamous cell carcinoma (basaloid, clear cell, papillary, small cell), large cell (undifferentiated) carcinoma (giant cell, basaloid, clear cell, large cell [with rhabdoid phenotype], large cell neuroendocrine carcinoma [LCNEC], combined LCNEC); small cell lung cancer including small cell (oat cell) carcinoma, combined small cell; adenoid cystic carcinoma; hamartoma; lymphoma; neuroendocrine/carcinoid; sarcoma. Gastrointestinal: esophagus (squamous cell carcinoma, larynx, adenocarcinoma, leiomyosarcoma, lymphoma), stomach (carcinoma, lymphoma, leiomyosarcoma), pancreas (ductal adenocarcinoma, insulinoma, glucagonoma, gastrinoma, carcinoid tumors, vipoma), small bowel or small intestines (adenocarcinoma, lymphoma, carcinoid tumors, Karposi's sarcoma, leiomyoma, hemangioma, lipoma, neurofibroma, fibroma), large bowel or large intestines (adenocarcinoma, tubular adenoma, villous adenoma, hamartoma, leiomyoma), colon, colon-rectum, colorectal; rectum, Genitourinary tract: kidney (adenocarcinoma, Wilm's tumor [nephroblastoma], lymphoma, leukemia), bladder and urethra (squamous cell carcinoma, transitional cell carcinoma, adenocarcinoma), prostate (adenocarcinoma, sarcoma), testis (seminoma, teratoma, embryonal carcinoma, teratocarcinoma, choriocarcinoma, sarcoma, interstitial cell carcinoma, fibroma, fibroadenoma, adenomatoid tumors, lipoma); Liver: hepatoma (hepatocellular carcinoma), cholangiocarcinoma, hepatoblastoma, angiosarcoma, hepatocellular adenoma, hemangioma, biliary passages; Bone: osteogenic sarcoma (osteosarcoma), fibrosarcoma, malignant fibrous histiocytoma, chondrosarcoma, Ewing's sarcoma, malignant lymphoma (reticulum cell sarcoma), multiple myeloma, malignant giant cell tumor chordoma, osteochronfroma (osteocartilaginous exostoses), benign chondroma, chondroblastoma, chondromyxofibroma, osteoid osteoma and giant cell tumors; Nervous system: skull (osteoma, hemangioma, granuloma, xanthoma, osteitis deformans), meninges (meningioma, meningiosarcoma, gliomatosis), brain (astrocytoma, medulloblastoma, glioma, ependymoma, germinoma [pinealoma], glioblastoma multiform, oligodendroglioma, schwannoma, retinoblastoma, congenital tumors), spinal cord neurofibroma, meningioma, glioma, sarcoma); Female/Gynecological: uterus (endometrial carcinoma), cervix (cervical carcinoma, pre-tumor cervical dysplasia), ovaries (ovarian carcinoma [serous cystadenocarcinoma, mucinous cystadenocarcinoma, unclassified carcinoma], granulosa-thecal cell tumors, Sertoli-Leydig cell tumors, dysgerminoma, malignant teratoma), vulva (squamous cell carcinoma, intraepithelial carcinoma, adenocarcinoma, fibrosarcoma, melanoma), vagina (clear cell carcinoma, squamous cell carcinoma, botryoid sarcoma (embryonal rhabdomyosarcoma), fallopian tubes (carcinoma), breast; Hematologic: blood (myeloid leukemia [acute and chronic], acute lymphoblastic leukemia, chronic lymphocytic leukemia, myeloproliferative diseases, multiple myeloma, myelodysplastic syndrome), Hodgkin's disease, non-Hodgkin's lymphoma [malignant lymphoma] hairy cell; lymphoid disorders; Skin: malignant melanoma, basal cell carcinoma, squamous cell carcinoma, Karposi's sarcoma, keratoacanthoma, moles dysplastic nevi, lipoma, angioma, dermatofibroma, keloids, psoriasis, Thyroid gland: papillary thyroid carcinoma, follicular thyroid carcinoma, undifferentiated thyroid cancer, medullary thyroid carcinoma, multiple endocrine neoplasia type 2A, multiple endocrine neoplasia type 2B, familial medullary thyroid cancer, pheochromocytoma, paraganglioma; and Adrenal glands: neuroblastoma.

In some embodiments, the cancer is selected from head and neck, ovarian, melanoma cervical, endometrial, esophageal, or breast cancer.

In certain embodiments, the term “myeloproliferative disorders”, includes disorders such as polycythemia vera, thrombocythemia, myeloid metaplasia with myelofibrosis, hypereosinophilic syndrome, juvenile myelomonocytic leukemia, systemic mast cell disease, and hematopoietic disorders, in particular, acute-myelogenous leukemia (AML), chronic-myelogenous leukemia (CML), acute-promyelocytic leukemia (APL), and acute lymphocytic leukemia (ALL).

In various embodiments, the cancer is a metastatic or locally advanced unresectable solid tumor. In some embodiments, the cancer is selected from malignant melanoma, acute myelogenous leukemia, pancreatic, colorectal, lung, prostate, cervical, brain, liver, head and neck, endometrial, esophageal, breast, and ovarian cancers, and histological subtypes thereof. In a preferred embodiment, the cancer is colorectal cancer. Colorectal cancer (CRC) can be subdivided into several molecular subgroups based on, e.g., KRAS and NRAS mutational status, which has an impact on treatment (e.g., EGFR targeting vs. VEGF targeting). Another characterization is based on the microsatelite status, either stable (MSS) or instable, either low (MSI-L) or high (MSI-H). MSI-H is seen in only ˜15% of all patients with CRC but MSI-L/MSS in 85%. Earlier studies have shown that PD-x in monotherapy have no effect on MSS/MSI-L CRC patients (0% ORR) (Le et al. (2015), N Engl J Med 372: 2509).

In various embodiments, the method of the invention is employed as a first, second, third or later line of treatment. A line of treatment refers to a place in the order of treatment with different medications or other therapies received by a patient. First-line therapy regimens are treatments given first, whereas second- or third-line therapy is given after the first-line therapy or after the second-line therapy, respectively. Therefore, first-line therapy is the first treatment for a disease or condition. In patients with cancer, first-line therapy, sometimes referred to as primary therapy or primary treatment, can be surgery, chemotherapy, radiation therapy, or a combination of these therapies. Typically, a patient is given a subsequent chemotherapy regimen (second- or third-line therapy), either because the patient did not show a positive clinical outcome or only showed a sub-clinical response to a first- or second-line therapy or showed a positive clinical response but later experienced a relapse, sometimes with disease now resistant to the earlier therapy that elicited the earlier positive response.

The safety and the clinical benefit offered by the therapeutic combination of the invention warrants a first-line setting in cancer patients. Particularly, the combination may become a new standard treatment for patients suffering from a cancer. In another embodiment of the invention, the therapeutic combination of the invention is applied in a later line of treatment, particularly a second-line or higher treatment of the cancer. There is no limitation to the prior number of therapies provided that the subject underwent at least one round of prior cancer therapy. The round of prior cancer therapy refers to a defined schedule/phase for treating a subject with, e.g., one or more immunotherapeutic agents (e.g., an anti-PD-L1 antibody), chemotherapeutic agents (excluding IDO1 inhibitors), radiotherapy or chemoradiotherapy, and the subject failed with such previous treatment, which was either completed or terminated ahead of schedule. One reason could be that the cancer was resistant or became resistant to prior therapy. The addition of the IDO1 inhibitor will suppress this mechanism of resistance and restore the effect of the immunotherapy. The set of patients with resistance becomes treatable and shows improved responses.

As the mode of action of the IDO1 inhibitor is different from that of the anti-PD-L1 antibodies, the chances to have enhanced immune-related adverse events (irAE) are small although both agents are targeting the immune system. The absence of overlapping immune features in nonclinical findings or in published clinical results makes the risk low for the combination therapy of the invention to show enhanced adverse events above what is generally observed in the class of PD-1/PD-L1 targeting agents. The clinical experience of IDO inhibitors (e.g., epacadostat, indoximod or GDC-0919) combined with pembrolizumab, nivolumab and atezolizumab in multiple Phase I/II trials have demonstrated tolerable safety profiles similar to that of single agent IDO inhibitors as well as that of anti-PD-1/PD-L1 (see e.g., Burris et al. (2017) J Clin Oncol 35: Suppl abstr 105; Gangadhar et al. (2017) J Clin Oncol 35: Suppl abstr 9014; Hamid et al. (2017) J Clin Oncol 35: Suppl abstr 3012 and 6010; Perez et al. (2017) J Clin Oncol 35: Suppl abstr 3003; Smith et al. (2017) J Clin Oncol 35: Suppl abstr 4503). The identified and potential risks for the anti-PD-L1 antibody of the invention, preferably avelumab, and for the IDO1 inhibitor of the invention, preferably Compound 1 or 2, as single agents are considered the potential risks for the combination treatment.

The current standard of care (SoC) for treating cancer patients often involves the administration of toxic and old chemotherapy regimens. The SoC is associated with high risks of strong adverse events that are likely to interfere with the quality of life (such as secondary cancers). The toxicity profile of an anti-PD-L1 antibody/IDO1 inhibitor combination, preferably avelumab and Compound 1 or 2, or a pharmaceutically acceptable salt thereof, seems to be much better than the SoC chemotherapy. In one embodiment, an anti-PD-L1 antibody/IDO1 inhibitor combination, preferably avelumab and Compound 1 or 2, or a pharmaceutically acceptable salt thereof, may be as effective and better tolerated than SoC chemotherapy in patients with cancer resistant to mono- and/or poly-chemotherapy, radiotherapy or chemoradiotherapy.

In some embodiments that employ an anti-PD-L1 antibody in the combination therapy, the dosing regimen will comprise administering the anti-PD-L1 antibody at a dose of about 1, 2, 3, 4, 5, 6, 7, 8, 9, 10, 11, 12, 13, 14, 15, 16, 17, 18, 19 or 20 mg/kg at intervals of about 14 days (±2 days) or about 21 days (±2 days) or about 30 days (±2 days) throughout the course of treatment. In other embodiments that employ an anti-PD-L1 antibody in the combination therapy, the dosing regimen will comprise administering the anti-PD-L1 antibody at a dose of from about 0.005 mg/kg to about 10 mg/kg, with intra-patient dose escalation. In other escalating dose embodiments, the interval between doses will be progressively shortened, e.g., about 30 days (±2 days) between the first and second dose, about 14 days (±2 days) between the second and third doses. In certain embodiments, the dosing interval will be about 14 days (±2 days), for doses subsequent to the second dose. In certain embodiments, a subject will be administered an intravenous (IV) infusion of a medicament comprising any of the anti-PD-L1 antibody described herein. In some embodiments, the anti-PD-L1 antibody in the combination therapy is avelumab, which is administered intravenously at a dose selected from the group consisting of: about 1 mg/kg Q2W (Q2W=one dose every two weeks), about 2 mg/kg Q2W, about 3 mg/kg Q2W, about 5 mg/kg Q2W, about 10 mg/kg Q2W, about 1 mg/kg Q3W (Q3W=one dose every three weeks), about 2 mg/kg Q3W, about 3 mg/kg Q3W, about 5 mg/kg Q3W, and about 10 mg Q3W. In some embodiments of the invention, the anti-PD-L1 antibody in the combination therapy is avelumab, which is administered in a liquid medicament at a dose selected from the group consisting of about 1 mg/kg Q2W, about 2 mg/kg Q2W, about 3 mg/kg Q2W, about 5 mg/kg Q2W, about 10 mg Q2W, about 1 mg/kg Q3W, about 2 mg/kg Q3W, about 3 mg/kg Q3W, about 5 mg/kg Q3W, and about 10 mg Q3W. In some embodiments, a treatment cycle begins with the first day of combination treatment and last for 2 weeks. In such embodiments, the combination therapy is preferably administered for at least 12 weeks (6 cycles of treatment), more preferably at least 24 weeks, and even more preferably at least 2 weeks after the patient achieves a CR. In some embodiment, avelumab is administered as a flat dose of about 80, 150, 160, 200, 240, 250, 300, 320, 350, 400, 450, 480, 500, 550, 560, 600, 640, 650, 700, 720, 750, 800, 850, 880, 900, 950, 960, 1000, 1040, 1050, 1100, 1120, 1150, 1200, 1250, 1280, 1300, 1350, 1360, 1400, 1440, 1500, 1520, 1550 or 1600 mg, preferably 800 mg, 1200 mg or 1600 mg at intervals of about 14 days (±2 days) or about 21 days (±2 days) or about 30 days (±2 days) throughout the course of treatment.

In another embodiment, the anti-PD-L1 antibody, preferably avelumab, will be given IV every two week. In certain embodiments, the anti-PD-L1 antibody is administered intravenously for 50-80 minutes at a dose of about 10 mg/kg body weight every two weeks. In a more preferred embodiment, the avelumab dose will be 10 mg/kg body weight administered as 1-hour intravenous infusions every 2 weeks (Q2W). Given the variability of infusion pumps from site to site, a time window of minus 10 minutes and plus 20 minutes is permitted. Pharmacokinetic studies demonstrated that the 10 mg/kg dose of avelumab achieves excellent receptor occupancy with a predictable pharmacokinetics profile (see e.g., Heery et al. (2015) Proc ASCO Annual Meeting: abstract 3055). This dose is well tolerated, and signs of antitumor activity, including durable responses, have been observed. Avelumab may be administered up to 3 days before or after the scheduled day of administration of each cycle due to administrative reasons.

In some embodiments, provided methods comprise administering a pharmaceutically acceptable composition comprising the IDO1 inhibitor, preferably Compound 1 or 2, or a pharmaceutically acceptable salt thereof, one, two, three or four times a day. In some embodiments, a pharmaceutically acceptable composition comprising the IDO1 inhibitor, preferably Compound 1 or 2, or a pharmaceutically acceptable salt thereof, is administered once daily (“QD”), particularly continuously. In some embodiments, a pharmaceutically acceptable composition comprising the IDO1 inhibitor, preferably Compound 1 or 2, or a pharmaceutically acceptable salt thereof, is administered twice daily, particularly continuously. In some embodiments, twice daily administration refers to a compound or composition that is administered “BID”, or two equivalent doses administered at two different times in one day. In some embodiments, a pharmaceutically acceptable composition comprising the IDO1 inhibitor, preferably Compound 1 or 2, or a pharmaceutically acceptable salt thereof, is administered three times a day. In some embodiments, a pharmaceutically acceptable composition comprising Compound 1 or 2, or a pharmaceutically acceptable salt thereof, is administered “TID”, or three equivalent doses administered at three different times in one day. In some embodiments, a pharmaceutically acceptable composition comprising the IDO1 inhibitor, preferably Compound 1 or 2, or a pharmaceutically acceptable salt thereof, is administered four times a day. In some embodiments, a pharmaceutically acceptable composition comprising Compound 1 or 2, or a pharmaceutically acceptable salt thereof, is administered “QD”, or four equivalent doses administered at four different times in one day. In some embodiments, the IDO1 inhibitor, preferably Compound 1 or 2, or a pharmaceutically acceptable salt thereof, is administered to a patient under fasted conditions and the total daily dose is any of those contemplated above and herein. In some embodiments, the IDO1 inhibitor, preferably Compound 1 or 2, or a pharmaceutically acceptable salt thereof, is administered to a patient under fed conditions and the total daily dose is any of those contemplated above and herein. In some embodiments, the IDO1 inhibitor, preferably Compound 1 or 2, or a pharmaceutically acceptable salt thereof, is administered orally. In some embodiments, the IDO1 inhibitor, preferably Compound 1 or 2, or a pharmaceutically acceptable salt thereof, will be given orally twice daily. In preferred embodiments, the IDO1 inhibitor, particularly Compound 1 or 2, or a pharmaceutically acceptable salt thereof, is administered twice daily (BID), at a dose of about 0.01 to about 1000 mg/kg, particularly at a dose of about 0.01 to about 200 mg/kg. In other preferred embodiments, the IDO1 inhibitor, particularly Compound 1 or 2, or a pharmaceutically acceptable salt thereof, is administered twice daily (BID), at a dose of about 10 to about 1000 mg, particularly at a dose of about 100 to about 900 mg. In more preferred embodiments, the IDO1 inhibitor, preferably Compound 1 or 2, or a pharmaceutically acceptable salt thereof, is administered twice daily (BID) for 3 to 4 weeks, at a dose of about 0.01 to about 1000 mg/kg, particularly at a dose of about 0.01 to about 200 mg/kg. In other preferred embodiments, the IDO1 inhibitor, particularly Compound 1 or 2, or a pharmaceutically acceptable salt thereof, is administered twice daily (BID) for 3 to 4 weeks, at a dose of about 10 to about 1000 mg, particularly at a dose of about 100 to about 900 mg.

In certain embodiments, the invention provides a method treatment, as described above, further comprising an additional step of administering to said patient an additional therapeutic agent is selected from a chemotherapeutic or anti-proliferative agent, an anti-inflammatory agent, an immunomodulatory or immunosuppressive agent, a neurotrophic factor, an agent for treating cardiovascular disease, an agent for treating destructive bone disorders, an anti-viral agent, an agent for treating blood disorders, or an agent for treating immunodeficiency disorders, wherein said additional therapeutic agent is appropriate for the disease being treated.

Concurrent treatment considered necessary for the patient's well-being may be given at discretion of the treating physician. In some embodiments, the anti-PD-L1 antibody and IDO1 inhibitor are administered in combination with chemotherapy (CT), radiotherapy (RT), or chemotherapy and radiotherapy (CRT). As described herein, in some embodiments, the present invention provides methods of treating, stabilizing or lessening the severity or progression of one or more diseases or disorders associated with PD-L1 and IDO1 comprising administering to a patient in need thereof an anti-PD-L1 antibody and an IDO1 inhibitor in combination with an additional chemotherapeutic agent. In certain embodiments, the chemotherapeutic agent is selected from the group of etoposide, topotecan, irinotecan, fluorouracil, a platin, an anthracycline, and a combination thereof.

In certain embodiments, the additional chemotherapeutic agent is topotecan, etoposide and/or anthracycline treatment, either as single cytostatic agent or as part of a doublet or triplet regiment. With such a chemotherapy, the IDO1 inhibitor can be preferably given once or twice daily with the anti-PD-L1 antibody, particularly avelumab, which is given every second week. In cases, in which anthracyclines are used, the treatment with anthracycline is stopped once a maximal life-long accumulative dose has been reached (due to the cardiotoxicity).

In certain embodiments, the additional chemotherapeutic agent is a platin. Platins are platinum-based chemotherapeutic agents. As used herein, the term “platin” is used interchangeably with the term “platinating agent.” Platinating agents are well known in the art. In some embodiments, the platin (or platinating agent) is selected from cisplatin, carboplatin, oxaliplatin, nedaplatin, and satraplatin. In some embodiments, the additional chemotherapeutic is a combination of both of etoposide and a platin. In certain embodiments, the platin is cisplatin. In certain embodiments, the provided method further comprises administration of radiation therapy to the patient. In some embodiments, the additional chemotherapeutic is a combination of both of etoposide and cisplatin.

In certain embodiments, the additional therapeutic agent is selected from daunomycin, doxorubicin, epirubicin, idarubicin, valrubicin, mitoxantrone, paclitaxel, docetaxel and cyclophosphamide.

In other embodiments, the additional therapeutic agent is selected from a CTLA4 agent (e.g., ipilimumab (BMS)); GITR agent (e.g., MK-4166 (MSD)); vaccines (e.g., sipuleucel-t (Dendron); or a SoC agent (e.g., radiation, docetaxel, temozolomide (MSD), gemcitibine or paclitaxel). In other embodiments, the additional therapeutic agent is an immune enhancer such as a vaccine, immune-stimulating antibody, immunoglobulin, agent or adjuvant including, but not limited to, sipuleucel-t, BMS-663513 (BMS), CP-870893 (Pfizer/VLST), anti-OX40 (AgonOX), or CDX-1127 (CellDex).

Other cancer therapies or anti-cancer agents that may be used in combination with the inventive agents of the present invention include surgery, radiotherapy (e.g., gamma-radiation, neutron beam radiotherapy, electron beam radiotherapy, proton therapy, brachytherapy, low-dose radiotherapy, and systemic radioactive isotopes), immune response modifiers such as chemokine receptor antagonists, chemokines and cytokines (e.g., interferons, interleukins, tumor necrosis factor (TNF), and GM-CSF)), hyperthermia and cryotherapy, agents to attenuate any adverse effects (e.g. antimetics, steroids, anti-inflammatory agents), and other approved chemotherapeutic drugs.

In certain embodiments, the additional therapeutic agent is selected from an antibiotic, a vasopressor, a steroid, an inotrope, an anti-thrombotic agent, a sedative, opioids or an anesthetic.

In certain embodiments, the additional therapeutic agent is selected from cephalosporins, macrolides, penams, beta-lactamase inhibitors, aminoglycoside antibiotics, fluoroquinolone antibiotics, glycopeptide antibiotics, penems, monobactams, carbapenmems, nitroimidazole antibiotics, lincosamide antibiotics, vasopressors, positive inotropic agents, steroids, benzodiazepines, phenol, alpha2-adrenergic receptor agonists, GABA-A receptor modulators, anti-thrombotic agents, anesthetics or opiods.

The IDO1 inhibitor, preferably Compound 1 or 2, or a pharmaceutically acceptable salt thereof, and compositions thereof in combination with the anti-PD-L1 antibody and additional chemotherapeutic according to methods of the present invention, are administered using any amount and any route of administration effective for treating or lessening the severity of a disorder provided above. The exact amount required will vary from subject to subject, depending on the species, age, and general condition of the subject, the severity of the infection, the particular agent, its mode of administration, and the like.

In some embodiments, provided methods comprise administering a pharmaceutically acceptable composition comprising a chemotherapeutic agent one, two, three or four times a day. In some embodiments, a pharmaceutically acceptable composition comprising a chemotherapeutic agent is administered once daily (“QD”). In some embodiments, a pharmaceutically acceptable composition comprising a chemotherapeutic agent is administered twice daily. In some embodiments, twice daily administration refers to a compound or composition that is administered “BID”, or two equivalent doses administered at two different times in one day. In some embodiments, a pharmaceutically acceptable composition comprising a chemotherapeutic agent is administered three times a day. In some embodiments, a pharmaceutically acceptable composition comprising a chemotherapeutic agent is administered “TID”, or three equivalent doses administered at three different times in one day. In some embodiments, a pharmaceutically acceptable composition comprising a chemotherapeutic agent is administered four times a day. In some embodiments, a pharmaceutically acceptable composition comprising a chemotherapeutic agent is administered “QID”, or four equivalent doses administered at four different times in one day. In some embodiments, a pharmaceutically acceptable composition comprising a chemotherapeutic agent is administered for a various number of days (for example 14, 21, 28) with a various number of days between treatment (0, 14, 21, 28). In some embodiments, a chemotherapeutic agent is administered to a patient under fasted conditions and the total daily dose is any of those contemplated above and herein. In some embodiments, a chemotherapeutic agent is administered to a patient under fed conditions and the total daily dose is any of those contemplated above and herein. In some embodiments, a chemotherapeutic agent is administered orally for reasons of convenience. In some embodiments, when administered orally, a chemotherapeutic agent is administered with a meal and water. In another embodiment, the chemotherapeutic agent is dispersed in water or juice (e.g., apple juice or orange juice) and administered orally as a suspension. In some embodiments, when administered orally, a chemotherapeutic agent is administered in a fasted state. A chemotherapeutic agent can also be administered intradermally, intramuscularly, intraperitoneally, percutaneously, intravenously, subcutaneously, intranasally, epidurally, sublingually, intracerebrally, intravaginally, transdermally, rectally, mucosally, by inhalation, or topically to the ears, nose, eyes, or skin. The mode of administration is left to the discretion of the health-care practitioner, and can depend in-part upon the site of the medical condition.

In certain embodiments, the anti-PD-L1 antibody and IDO1 inhibitor, preferably Compound 1 or 2, or a pharmaceutically acceptable salt thereof, are administered in combination with radiotherapy. In certain embodiments, the radiotherapy comprises about 35-70 Gy/20-35 fractions. In some embodiments, the radiotherapy is given either with standard fractionation (1.8 to 2 Gy for day 5 days a week) up to a total dose of 50-70 Gy in once daily. Other fractionation schedules could also be envisioned, for example, a lower dose per fraction but given twice daily with the IDO1 inhibitor given also twice daily. Higher daily doses over a shorter period of time can also be given. In one embodiment, stereotactic radiotherapy as well as the gamma knife are used. In the palliative setting, other fractionation schedules are also widely used for example 25 Gy in 5 fractions or 30 Gy in 10 fractions. In all cases, avelumab is preferably given every second week. For radiotherapy, the duration of treatment will be the time frame when radiotherapy is given. These interventions apply to treatment given with electrons, photons and protons, alfa-emitters or other ions, treatment with radio-nucleotides, for example, treatment with ¹³¹I given to patients with thyroid cancer, as well in patients treated with boron capture neutron therapy.

In some embodiments, the anti-PD-L1 antibody and IDO1 inhibitor are administered simultaneously, separately or sequentially and in any order. The anti-PD-1 antibody and IDO1 inhibitor are administered to the patient in any order (i.e., simultaneously or sequentially) in separate compositions, formulations or unit dosage forms, or together in a single composition, formulation or unit dosage form. In one embodiment, a method of treating a proliferative disease may comprise administration of a combination of an IDO1 inhibitor and an anti PD-L1 antibody, wherein the individual combination partners are administered simultaneously or sequentially in any order, in jointly therapeutically effective amounts, (for example in synergistically effective amounts), e.g. in daily or intermittently dosages corresponding to the amounts described herein. The individual combination partners of a combination therapy of the invention may be administered separately at different times during the course of therapy or concurrently in divided or single combination forms. Typically, in such combination therapies, the first active component which is at least one IDO1 inhibitor, and the anti-PD-L1 antibody are formulated into separate pharmaceutical compositions or medicaments. When separately formulated, the at least two active components can be administered simultaneously or sequentially, optionally via different routes. Optionally, the treatment regimens for each of the active components in the combination have different but overlapping delivery regimens, e.g., daily, twice daily, vs. a single administration, or weekly. The second active component (anti-PD-L1 antibody) may be delivered prior to, substantially simultaneously with, or after, the at least one IDO1 inhibitor. In certain embodiments, the anti-PD-1 antibody is administered simultaneously in the same composition comprising the anti-PD-L1 antibody and IDO1 inhibitor. In certain embodiments, the anti-PD-L1 antibody and IDO1 inhibitor are administered simultaneously in separate compositions, i.e., wherein the anti-PD-L1 antibody and IDO1 inhibitor are administered simultaneously each in a separate unit dosage form. It will be appreciated that the anti-PD-1 antibody and IDO1 inhibitor are administered on the same day or on different days and in any order as according to an appropriate dosing protocol. The instant invention is therefore to be understood as embracing all such regimens of simultaneous or alternating treatment and the term “administering” is to be interpreted accordingly.

In some embodiments, the method comprises the steps of: (a) under the direction or control of a physician, the subject receiving the PD-L1 antibody prior to first receipt of the IDO1 inhibitor; and (b) under the direction or control of a physician, the subject receiving the IDO1 inhibitor. In some embodiments, the method comprises the steps of: (a) under the direction or control of a physician, the subject receiving the IDO1 inhibitor prior to first receipt of the PD-L1 antibody; and (b) under the direction or control of a physician, the subject receiving the PD-L1 antibody. In some embodiments, the method comprises the steps of: (a) prescribing the subject to self-administer, and verifying that the subject has self-administered, the PD-L1 antibody prior to first administration of the IDO1 inhibitor; and (b) administering the IDO1 inhibitor to the subject. In some embodiments, the method comprises the steps of: (a) prescribing the subject to self-administer, and verifying that the subject has self-administered, the IDO1 inhibitor prior to first administration of the PD-L1 antibody; and (b) administering the PD-L1 antibody to the subject. In some embodiments, the method comprises, after the subject has received the PD-L1 antibody prior to the first administration of the IDO1 inhibitor, administering the IDO1 inhibitor to the subject. In some embodiments, the method comprises the steps of: (a) after the subject has received the PD-L1 antibody prior to the first administration of the IDO1 inhibitor, determining that an IDO1 level in a cancer sample isolated from the subject exceeds an IDO1 level predetermined prior to the first receipt of the anti-PD-L1 antibody, and (b) administering the IDO1 inhibitor to the subject. In some embodiments, the method comprises the steps of: (a) after the subject has received prior cancer therapy and/or the PD-L1 antibody prior to the first administration of the IDO1 inhibitor, determining that an IDO1 level in a cancer sample isolated from the subject exceeds an IDO1 level predetermined prior to the first receipt of the prior cancer therapy and/or the anti-PD-L1 antibody, and (b) administering the IDO1 inhibitor to the subject. In some embodiments, the method comprises, after the subject has received the IDO1 inhibitor prior to first administration of the anti-PD-L1 antibody, administering the anti-PD-L1 antibody to the subject.

Also provided herein is an anti-PD-L1 antibody for use as a medicament in combination with an IDO1 inhibitor. Similarly provided is an IDO1 inhibitor for use as a medicament in combination with an anti-PD-L1 antibody. Also provided is an anti-PD-L1 antibody for use in the treatment of cancer in combination with an IDO1 inhibitor. Similarly provided is an IDO1 inhibitor for use in the treatment of cancer in combination with an anti-PD-L1 antibody. One aspect of the invention also provides an anti-PD-L1 antibody for use in the treatment of an IDO1-positive cancer, which induces an escape pathway to checkpoint inhibitor treatment, in combination with an IDO1 inhibitor. Similarly provided is an IDO1 inhibitor for use in the treatment of an IDO1-positive cancer, which induces an escape pathway to checkpoint inhibitor treatment, in combination with an anti-PD-L1 antibody. In various embodiments above, the IDO1 inhibitor 3-(5-fluoro-1H-indol-3-yl)pyrrolididine-2,5-dione is excluded.

Also provided is a combination comprising an anti-PD-L1 antibody and an IDO1 inhibitor. Also provided is a combination comprising an anti-PD-L1 antibody and an IDO1 inhibitor for use as a medicament. Also provided is a combination comprising an anti-PD-L1 antibody and an IDO1 inhibitor for the use in the treatment of cancer. The aforementioned combinations are provided in a single or separate unit dosage forms. In one aspect, the combination is used in the treatment of an IDO1-positive cancer that induces an escape pathway to checkpoint inhibitor treatment and excludes the IDO1 inhibitor 3-(5-fluoro-1H-indol-3-yl)pyrrolididine-2,5-dione.

Also provided is the use of a combination for the manufacture of a medicament for the treatment of cancer, comprising an anti-PD-L1 antibody and an IDO1 inhibitor.

The prior teaching of the present specification concerning the therapeutic combination, including the methods of using it, and all aspects and embodiments thereof, of this Section titled “Therapeutic combination and method of use thereof” is valid and applicable without restrictions to the medicament, the anti-PD-L1 antibody and/or IDO1 inhibitor for use in the treatment of cancer as well as the combination, and aspects and embodiments thereof, of this Section, if appropriate.

### Pharmaceutical Formulations and Kits

In some embodiments, the present invention provides a pharmaceutically acceptable composition comprising an anti-PD-L1 antibody. In some embodiments, the present invention provides a pharmaceutically acceptable composition comprising an IDO1 inhibitor, preferably Compound 1 or 2, or a pharmaceutically acceptable salt thereof. In some embodiments, the present invention provides a pharmaceutically acceptable composition of a chemotherapeutic agent. In some embodiments, the present invention provides a pharmaceutical composition comprising an anti-PD-L1 antibody, an IDO1 inhibitor and at least a pharmaceutically acceptable excipient or adjuvant. The aforementioned pharmaceutical compositions of the anti-PD-L1 antibody and the IDO1 inhibitor are provided in a single or separate unit dosage forms. In various embodiments described above and below, the anti-PD-L1 antibody comprises a heavy chain, which comprises three complementarity determining regions having amino acid sequences of SEQ ID NOs: 1, 2 and 3, and a light chain, which comprises three complementarity determining regions having amino acid sequences of SEQ ID NOs: 4, 5 and 6. In some embodiments, a composition comprising an IDO1 inhibitor, preferably Compound 1 or 2, or a pharmaceutically acceptable salt thereof, is separate from a composition comprising an anti-PD-L1 antibody and/or a chemotherapeutic agent. In some embodiments, an IDO1 inhibitor, preferably Compound 1 or 2, or a pharmaceutically acceptable salt thereof, and an anti-PD-L1 antibody and/or a chemotherapeutic agent are present in the same composition. Exemplary pharmaceutically acceptable compositions are described further below and herein.

According to another embodiment, the invention provides a composition comprising an IDO1 inhibitor of this invention or a pharmaceutically acceptable derivative thereof and a pharmaceutically acceptable carrier, adjuvant or vehicle. The amount of IDO1 inhibitor in compositions of this invention is such that is effective to measurably modulate IDO1 in a biological sample or in a patient. In certain embodiments, the amount of IDO1 inhibitor in compositions of this invention is such that is effective to measurably modulate IDO1 in a biological sample or in a patient. In certain embodiments, a composition of this invention is formulated for administration to a patient in need of such composition.

Pharmaceutically acceptable carriers, adjuvants or vehicles that are used in the compositions of this invention include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

Compositions of the present invention are administered orally, parenterally, by inhalation spray, topically, rectally, nasally, buccally, vaginally or via an implanted reservoir. The term “parenteral” as used herein includes subcutaneous, intravenous, intramuscular, intra-articular, intra-synovial, intrasternal, intrathecal, intrahepatic, intralesional and intracranial injection or infusion techniques. Preferably, the compositions are administered orally, intraperitoneally or intravenously.

Most preferably, pharmaceutically acceptable compositions of the IDO1 inhibitor are formulated for oral administration. Such formulations may be administered with or without food.

Typically, the anti-PD-L1 antibodies or antigen-binding fragments according to the invention are incorporated into pharmaceutical compositions suitable for administration to a subject, wherein the pharmaceutical composition comprises the anti-PD-L1 antibodies or antigen-binding fragments thereof, and a pharmaceutically acceptable carrier. In many cases, it is preferable to include isotonic agents, for example, sugars, polyalcohols such as mannitol, sorbitol, or sodium chloride in the composition. Pharmaceutically acceptable carriers may further comprise minor amounts of auxiliary substances such as wetting or emulsifying agents, preservatives or buffers, which enhance the shelf life or effectiveness of the anti-PD-L1 antibodies or antigen-binding fragments thereof.

The compositions of the present invention may be in a variety of forms. These include, for example, liquid, semi-solid and solid dosage forms, such as liquid solutions (e.g., injectable and infusible solutions), dispersions or suspensions, tablets, pills, powders, liposomes, and suppositories. The preferred form depends on the intended mode of administration and therapeutic application. Typical preferred compositions are in the form of injectable or infusible solutions, such as compositions similar to those used for passive immunization of humans. The preferred mode of administration is parenteral (e.g., intravenous, subcutaneous, intraperitoneal, or intramuscular). In a preferred embodiment, the anti-PD-L1 antibody or antigen-binding fragment thereof is administered by intravenous infusion or injection. In another preferred embodiment, the anti-PD-L1 antibody or antigen-binding fragment thereof is administered by intramuscular or subcutaneous injection.

Therapeutic compositions typically must be sterile and stable under the conditions of manufacture and storage. The composition can be formulated as a solution, microemulsion, dispersion, liposome, or other ordered structure suitable to high drug concentration. Sterile injectable solutions can be prepared by incorporating the active anti-PD-L1 antibody or antigen-binding fragment thereof in the required amount in an appropriate solvent with one or a combination of ingredients enumerated above, as required, followed by filtered sterilization. Generally, dispersions are prepared by incorporating the active ingredient into a sterile vehicle that contains a basic dispersion medium and the required other ingredients from those enumerated above. In the case of sterile powders for the preparation of sterile injectable solutions, the preferred methods of preparation are vacuum drying and freeze-drying that yield a powder of the active ingredient plus any additional desired ingredient from a previously sterile-filtered solution thereof. The proper fluidity of a solution can be maintained, for example, by the use of a coating such as lecithin, by the maintenance of the required particle size in the case of dispersion, and by the use of surfactants. Prolonged absorption of injectable compositions can be brought about by including in the composition an agent that delays absorption, for example, monostearate salts and gelatin.

In one embodiment, avelumab is a sterile, clear, and colorless solution intended for IV administration. The contents of the avelumab vials are non-pyrogenic, and do not contain bacteriostatic preservatives. Avelumab is formulated as a 20 mg/mL solution and is supplied in single-use glass vials, stoppered with a rubber septum and sealed with an aluminum polypropylene flip-off seal. For administration purposes, avelumab must be diluted with 0.9% sodium chloride (normal saline solution). Tubing with in-line, low protein binding 0.2 micron filter made of polyether sulfone (PES) is used during administration.

In a further aspect, the invention relates to a kit comprising an anti-PD-L1 antibody and a package insert comprising instructions for using the anti-PD-L1 antibody in combination with an IDO1 inhibitor to treat or delay progression of a cancer in a subject. Also provided is a kit comprising an IDO1 inhibitor and a package insert comprising instructions for using the IDO1 inhibitor in combination with an anti-PD-L1 antibody to treat or delay progression of a cancer in a subject. Also provided is a kit comprising an anti-PD-L1 antibody and an IDO1 inhibitor, and a package insert comprising instructions for using the anti-PD-L1 antibody and an IDO1 inhibitor to treat or delay progression of a cancer in a subject. The kit can comprise a first container, a second container and a package insert, wherein the first container comprises at least one dose of a medicament comprising the anti-PD-L1 antibody, the second container comprises at least one dose of a medicament comprising the IDO1 inhibitor, and the package insert comprises instructions for treating the subject for cancer using the medicaments. The first and second containers may be comprised of the same or different shape (e.g., vials, syringes and bottles) and/or material (e.g., plastic or glass). The kit may further comprise other materials that may be useful in administering the medicaments, such as diluents, filters, IV bags and lines, needles and syringes. The instructions can state that the medicaments are intended for use in treating a subject having a cancer that tests positive for PD-L1 or IDO1 expression, e.g., by means of an immunohistochemical (IHC) assay, FACS or LC/MS/MS. In various embodiments of the kit, the cancer is an IDO1-positive cancer that induces an escape pathway to checkpoint inhibitor treatment, wherein, optionally, the instructions can state that the medicaments are intended for use in treating a subject having a cancer that also tests positive for PD-L1 expression, e.g., by means of an immunohistochemical (IHC) assay. In various embodiments of the kit, the IDO1 inhibitor 3-(5-fluoro-1H-indol-3-yl)pyrrolididine-2,5-dione is excluded.

The prior teaching of the present specification concerning the therapeutic combination, including the methods of using it, and all aspects and embodiments thereof, of the previous Section titled “Therapeutic combination and method of use thereof” is valid and applicable without restrictions to the pharmaceutical formulations and kits, and aspects and embodiments thereof, of this Section titled “Pharmaceutical formulations and kits”, if appropriate.

Further Diagnostic, Predictive, Prognostic and/or Therapeutic Methods

The disclosure further provides diagnostic, predictive, prognostic and/or therapeutic methods, which are based, at least in part, on determination of the identity of the expression level of a marker of interest. In some embodiments, the amount of human PD-L1 in a cancer patient sample can be used to predict whether the patient is likely to respond favorably to cancer therapy utilizing the therapeutic combination of the invention. In some embodiments, the amount of human IDO1 in a cancer patient sample can be used to predict whether the patient is likely to respond favorably to cancer therapy utilizing the therapeutic combination of the invention. In some embodiments, the amount of tryptophan and/or kynurenine, or a modification of the ratio of tryptophan and/or kynurenine in a cancer patient sample, e.g. blood, can be used to predict whether the patient is likely to respond favorably to cancer therapy utilizing the therapeutic combination of the invention.

Any suitable sample can be used for the method. Non-limiting examples of such include one or more of a serum sample, plasma sample, whole blood, pancreatic juice sample, tissue sample, tumor lysate or a tumor sample, which can be an isolated from a needle biopsy, core biopsy and needle aspirate. For example, tissue, plasma or serum samples are taken from the patient before treatment and optionally on treatment with the therapeutic combination of the invention. The expression levels obtained on treatment are compared with the values obtained before starting treatment of the patient. The information obtained may be prognostic in that it can indicate whether a patient has responded favorably or unfavorably to cancer therapy.

It is to be understood that information obtained using the diagnostic assays described herein may be used alone or in combination with other information, such as, but not limited to, expression levels of other genes, clinical chemical parameters, histopathological parameters, or age, gender and weight of the subject. When used alone, the information obtained using the diagnostic assays described herein is useful in determining or identifying the clinical outcome of a treatment, selecting a patient for a treatment, or treating a patient, etc. When used in combination with other information, on the other hand, the information obtained using the diagnostic assays described herein is useful in aiding in the determination or identification of clinical outcome of a treatment, aiding in the selection of a patient for a treatment, or aiding in the treatment of a patient, and the like. In a particular aspect, the expression level can be used in a diagnostic panel each of which contributes to the final diagnosis, prognosis, or treatment selected for a patient.

Any suitable method can be used to measure the PD-L1 peptide or IDO1 protein, DNA, RNA, or other suitable read-outs for PD-L1 or IDO1 levels, examples of which are described herein and/or are well known to the skilled artisan (see e.g., Munn et al. (2012) Front Biosci. 4: 734).

In some embodiments, determining the PD-L1 or IDO1 level comprises determining the PD-L1 or IDO1 expression. In some preferred embodiments, the PD-L1 or IDO1 level is determined by the PD-L1 peptide concentration or IDO1 protein concentration in a patient sample, e.g., with PD-L1 or IDO1 specific ligands, such as antibodies or specific binding partners. The binding event can, e.g., be detected by competitive or non-competitive methods, including the use of a labeled ligand or PD-L1 or IDO1 specific moieties, e.g., antibodies, or labeled competitive moieties, including a labeled PD-L1 or IDO1 standard, which compete with marker proteins for the binding event. If the marker specific ligand is capable of forming a complex with PD-L1 or IDO1, the complex formation can indicate PD-L1 or IDO1 expression in the sample. In various embodiments, the biomarker protein level is determined by a method comprising quantitative western blot, multiple immunoassay formats, ELISA, immunohistochemistry, histochemistry, or use of FACS analysis of tumor lysates, immunofluorescence staining, a bead-based suspension immunoassay, Luminex technology, or a proximity ligation assay. In a preferred embodiment, the PD-L1 expression is determined by immunohistochemistry using one or more primary anti-PD-L1 antibodies. In another preferred embodiment, the IDO1 expression is determined by immunohistochemistry using one or more primary anti-IDO1 antibodies.

In another embodiment, the biomarker RNA level is determined by a method comprising microarray chips, RT-PCR, qRT-PCR, multiplex qPCR or in-situ hybridization. In one embodiment of the invention, a DNA or RNA array comprises an arrangement of poly-nucleotides presented by or hybridizing to the PD-L1 or IDO1 gene immobilized on a solid surface. For example, to the extent of determining the PD-L1 or IDO1 mRNA, the mRNA of the sample can be isolated, if necessary, after adequate sample preparation steps, e.g., tissue homogenization, and hybridized with marker specific probes, in particular on a microarray platform with or without amplification, or primers for PCR-based detection methods, e.g., PCR extension labeling with probes specific for a portion of marker mRNA. Several approaches have been described for quantifying PD-L1 peptide expression in IHC assays of tumor tissue sections (Thompson et al. (2004) PNAS 101(49): 17174; Thompson et al. (2006) Cancer Res. 66: 3381; Gadiot et al. (2012) Cancer 117: 2192; Taube et al. (2012) Sci Transl Med 4, 127ra37; and Toplian et al. (2012) New Eng. J Med. 366 (26): 2443). One approach employs a simple binary end-point of positive or negative for PD-L1 expression, with a positive result defined in terms of the percentage of tumor cells that exhibit histologic evidence of cell-surface membrane staining. A tumor tissue section is counted as positive for PD-L1 expression if it is at least 1%, and preferably 5% of total tumor cells.

The level of PD-L1 or IDO1 mRNA expression may be compared to the mRNA expression levels of one or more reference genes that are frequently used in quantitative RT-PCR, such as ubiquitin C. In some embodiments, a level of PD-L1 or IDO1 expression (protein and/or mRNA) by malignant cells and/or by infiltrating immune cells within a tumor is determined to be “overexpressed” or “elevated” based on comparison with the level of PD-L1 or IDO1 expression (protein and/or mRNA) by an appropriate control. For example, a control PD-L1 peptide or IDO1 protein or mRNA expression level may be the level quantified in non-malignant cells of the same type or in a section from a matched normal tissue.

In a preferred embodiment, the efficacy of the therapeutic combination of the invention is predicted by means of PD-L1 or IDO1 expression in tumor samples. Immunohistochemistry with anti-PD-L1 primary antibodies or anti-IDO1 primary antibodies can be performed on serial cuts of formalin fixed and paraffin embedded specimens from patients treated with an anti-PD-L1 antibody, such as avelumab, and an IDO1 inhibitor, such as Compound 1 or 2.

This disclosure also provides a kit for determining if the combination of the invention is suitable for therapeutic treatment of a cancer patient, comprising means for determining a protein level of PD-L1 or IDO1, or the expression level of its RNA, in a sample isolated from the patient and instructions for use. In another aspect, the kit further comprises avelumab for immunotherapy or Compound 2. In one aspect of the invention, the determination of a high PD-L1 or IDO1 level indicates increased PFS or OS when the patient is treated with the therapeutic combination of the invention. In one embodiment of the kit, the means for determining the PD-L1 peptide or IDO1 protein level are antibodies with specific binding to PD-L1 or IDO1, respectively.

In still another aspect, the invention provides a method for advertising an anti-PD-L1 antibody in combination with an IDO1 inhibitor, comprising promoting, to a target audience, the use of the combination for treating a subject with a cancer based on PD-L1 or IDO1 expression in samples taken from the subject. In still another aspect, the invention provides a method for advertising an IDO1 inhibitor in combination with an anti-PD-L1 antibody, comprising promoting, to a target audience, the use of the combination for treating a subject with a cancer based on PD-L1 or IDO1 expression in samples taken from the subject. In still another aspect, the invention provides a method for advertising a combination comprising an anti-PD-L1 antibody and an IDO1 inhibitor, comprising promoting, to a target audience, the use of the combination for treating a subject with a cancer based on PD-L1 or IDO1 expression in samples taken from the subject. Promotion may be conducted by any means available. In some embodiments, the promotion is by a package insert accompanying a commercial formulation of the therapeutic combination of the invention. The promotion may also be by a package insert accompanying a commercial formulation of the anti-PD-L1 antibody, IDO1 inhibitor or another medicament (when treatment is a therapy with the therapeutic combination of the invention and a further medicament). Promotion may be by written or oral communication to a physician or health care provider. In some embodiments, the promotion is by a package insert where the package insert provides instructions to receive therapy with the therapeutic combination of the invention after measuring PD-L1 or IDO1 expression levels, and in some embodiments, in combination with another medicament. In some embodiments, the promotion is followed by the treatment of the patient with the therapeutic combination of the invention with or without another medicament. In some embodiments, the package insert indicates that the therapeutic combination of the invention is to be used to treat the patient if the patient's cancer sample is characterized by high PD-L1 or IDO1 biomarker levels. In some embodiments, the package insert indicates that the therapeutic combination of the invention is not to be used to treat the patient if the patient's cancer sample expresses low PD-L1 or IDO1 biomarker levels. In some embodiments, a high PD-L1 or IDO1 biomarker level means a measured PD-L1 or IDO1 level that correlates with a likelihood of increased PFS and/or OS when the patient is treated with the therapeutic combination of the invention, and vice versa. In some embodiments, the PFS and/or OS is decreased relative to a patient who is not treated with the therapeutic combination of the invention. In some embodiments, the promotion is by a package insert where the package inset provides instructions to receive therapy with avelumab in combination with an IDO1 inhibitor after first measuring PD-L1 or IDO1 levels. In some embodiments, the promotion is followed by the treatment of the patient with avelumab in combination with an IDO1 inhibitor with or without another medicament. Further methods of advertising and instructing, or business methods applicable in accordance with the invention are described (for other drugs and biomarkers) in US 2012/0089541, for example.

In some embodiments, levels of tryptophan and/or kynurenine, or the ratio of tryptophan and kynurenine is used as a surrogate for the IDO1 expression. High-expressing IDO1 tumors were shown by IHC and the levels of tryptophan and kynurenine (see Examples). In these tumors, the levels of kynurenine increase dramatically upon addition of an anti-PD-L1 antibody, particularly an ADCC-mediating anti-PD-L1 antibody such as avelumab, and indicate the upregulation of IDO1. As avelumab does not perform well in monotherapy in high-expressing IDO1 tumors, it also confirms the theory, without being bound thereto, that upregulation of IDO1 is an escape mechanism to avelumab. Thus, IDO1 can be used as biomarker for a therapeutic anti-PD-L1 antibody.

It is another aspect to provide a method for predicting the likelihood that a subject suffering from a cancer, which is a candidate for treatment with an anti-PD-L1 antibody and an IDO1 inhibitor, will respond to the treatment, comprising determining the IDO1 expression by means of surrogate markers, which are tryptophan and kynurenine or the concentration ratio thereof, preferably by using LC/MS/MS, in a sample obtained from the subject, wherein a higher expression, as compared to a predetermined value, indicates that the subject is likely to respond to the treatment. The analytes in the worked-up samples are detected by tandem mass spectrometry following chromatographic separation (LC/MS/MS) and can be quantified based on calibration curve. In various embodiments of the method above, the cancer is an IDO1-positive cancer that induces an escape pathway to checkpoint inhibitor treatment, and the IDO1 inhibitor 3-(5-fluoro-1H-indol-3-yl)pyrrolididine-2,5-dione is excluded.

Thus, any of a variety of means can be used to assess surrogate levels in a patient or sample taken from the patient for the purpose of predicting whether the patient will respond favorably to the combination therapy according to the invention. If the surrogate level in the patient or patient sample is higher than or equal to a predetermined value for the surrogate level, the patient is administered the combination therapy, such as avelumab and Compound 2. A higher IDO1 expression correlates with an increase in kynurenine levels. The increase could be, e.g., at least 10%, 20%, 30%, 40% or 50%. The IDO1 inhibitors of the invention reduce kynurenine plasma levels by up to 50%. In some embodiments, the decrease is at least 10%, 20%, 30%, 40% or 50%. The doses that provide this effect on biomarker or pharmacodynamic readout are being used in clinical trials that show improved responses for the combination treatment of the invention. If the surrogate level is below that predetermined value, then the patient is administered an anti-cancer therapy other than the combination therapy of the invention.

It is still another aspect to provide a method for monitoring the likelihood of response to a treatment of a cancer, which is mediated and/or propagated by IDO1 expression, wherein a kynurenine plasma level is determined in a sample withdrawn from a subject in need of such treatment with an anti-PD-L1 antibody and an IDO1 inhibitor, wherein a decrease in the kynurenine plasma level relative to a predetermined level indicates an increased likelihood that the subject responds to the treatment with the anti-PD-L1 antibody and the IDO1 inhibitor. In various embodiments of the method above, the cancer is an IDO1-positive cancer that induces an escape pathway to checkpoint inhibitor treatment, and the IDO1 inhibitor 3-(5-fluoro-1H-indol-3-yl)pyrrolididine-2,5-dione is excluded. The present invention can be used as a clinical marker to monitor efficacy of the combination therapy on each patient individually. Specifically, kynurenine can be identified in a patient prior to an event, such as surgery, the onset of a therapeutic regime, or the completion of a therapeutic regime, to provide a base line result. This base-line can then be compared with the result obtained using identical methods either during or after such event. This information can be used for both diagnostic and prognostic purposes. The information about the clinical marker can be additionally used to optimize the dosage and the regimen of the combination treatment by monitoring the decrease of kynurenine in the subject's biological sample. Furthermore, the method of the present invention can be used to find a therapeutically effective combination regimen and/or a therapeutically effective amount for the selected combination, thereby individually selecting and optimizing a therapy for a patient.

A patient's likely clinical outcome following a clinical procedure such as a therapy or surgery can be expressed in relative terms. For example, a patient having a particular IDO1 or surrogate expression level who receives combination therapy may experience relatively longer overall survival than a patient or patients not having the IDO1 or surrogate expression level who receive combination therapy. The patient having the particular IDO1 or surrogate expression level, alternatively, can be considered as likely to survive if administered combination therapy. Similarly, a patient having a particular expression level who receives combination therapy may experience relatively longer progression free survival, or time to tumor progression, than a patient or patients not having the IDO1 or surrogate expression level who receive combination therapy. The patient having the particular IDO1 or surrogate expression level, alternatively, can be considered as not likely to suffer tumor progression if administered combination therapy. Further, a patient not having a particular IDO1 or surrogate expression level who receives combination therapy may experience relatively shorter time to tumor recurrence than a patient or patients having the expression level who receive combination therapy. The patient having the particular IDO1 or surrogate expression level, alternatively, can be considered as not likely to suffer tumor recurrence if administered combination therapy. It is still another example that a patient having a particular expression level if administered combination therapy may experience a relatively more complete response or partial response than a patient or patients not having the genotype or expression level who receive combination therapy. The patient having the particular genotype or expression level, alternatively, can be considered as likely to respond to combination therapy. Accordingly, a patient that is likely to survive, or not likely to suffer tumor progression, or not likely to suffer tumor recurrence, or likely to respond following a clinical procedure is considered suitable for the clinical procedure, treatment with a combination.

The prior teaching of the present specification concerning the therapeutic combination, including the methods of using it, and all aspects and embodiments thereof, of the previous Section titled “Therapeutic combination and method of use thereof” is valid and applicable without restrictions to the methods and kits, and aspects and embodiments thereof, of this Section titled “Further diagnostic, predictive, prognostic and/or therapeutic methods”, if appropriate.

All the references cited herein are incorporated by reference in the disclosure of the invention hereby.

It is to be understood that this invention is not limited to the particular molecules, pharmaceutical compositions, uses and methods described herein, as such matter can, of course, vary. It is also to be understood that the terminology used herein is for the purpose of describing particular embodiments only and is not intended to limit the scope of the present invention, which is only defined by the appended claims. The techniques that are essential according to the invention are described in detail in the specification. Other techniques which are not described in detail correspond to known standard methods that are well known to a person skilled in the art, or the techniques are described in more detail in cited references, patent applications or standard literature. Provided that no other hints in the application are given, they are used as examples only, they are not considered to be essential according to the invention, but they can be replaced by other suitable tools and biological materials.

Although methods and materials similar or equivalent to those described herein can be used in the practice or testing of the present invention, suitable examples are described below. Within the examples, standard reagents and buffers that are free from contaminating activities (whenever practical) are used. The examples are particularly to be construed such that they are not limited to the explicitly demonstrated combinations of features, but the exemplified features may be unrestrictedly combined again provided that the technical problem of the invention is solved. Similarly, the features of any claim can be combined with the features of one or more other claims. The present invention having been described in summary and in detail, is illustrated and not limited by the following examples.

### EXAMPLES

### Example 1: Synthesis of 4-fluoro-4-[2-[5H-imidazo[4,3-a]isoindol-5-yl]ethyl]cyclohexane-1-sulfonamide (121a, b, c, d)

All solvents used were commercially available and were used without further purification. Reactions were typically run using anhydrous solvents under an inert atmosphere of nitrogen.

All NMR experiments were recorded on a Bruker Mercury Plus 400 NMR Spectrometer equipped with a Bruker 400 BBFO probe at 400 MHz for proton NMR. All deuterated solvents contained typically 0.03% to 0.05% v/v tetramethylsilane, which was used as the reference signal (set at δ 0.00 for both ¹H and ¹³C).

LC-MS analyses were performed on a SHIMADZU LC-MS machine consisting of an UFLC 20-AD system and LCMS 2020 MS detector. The column used was a Shim-pack XR-ODS, 2.2 μm, 3.0×50 mm. A linear gradient was applied, starting at 95% A (A: 0.05% TFA in water) and ending at 100% B (B: 0.05% TFA in acetonitrile) over 2.2 min with a total run time of 3.6 min. The column temperature was at 40° C. with the flow rate at 1.0 mL/min. The Diode Array Detector was scanned from 200-400 nm. The mass spectrometer was equipped with an electro spray ion source (ES) operated in a positive or negative mode. The mass spectrometer was scanned between m/z 90-900 with a scan time of 0.6 s.

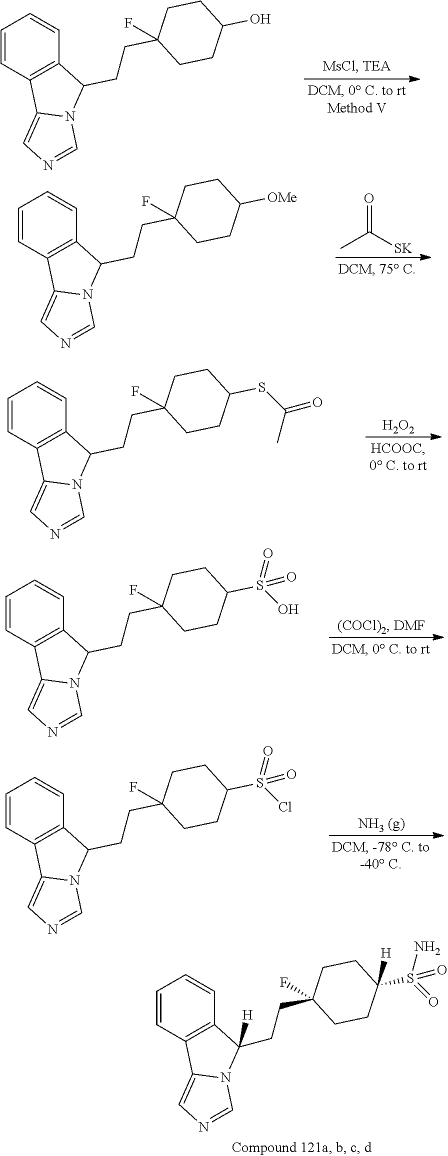

4-Fluoro-4-(2-[5H-imidazo[4,3-a]isoindol-5-yl]ethyl)cyclohexyl methanesulfonate: 4-Fluoro-4-(2-[5H-imidazo[4,3-a]isoindol-5-yl]ethyl)cyclohexyl methanesulfonate (1.6 g, 85%) was prepared from 4-fluoro-4-(2-[5H-imidazo[4,3-a]isoindol-5-yl]ethyl)cyclohexan-1-ol using Method V. MS: m/z=379.0 [M+H]⁺.

1-[[4-Fluoro-4-(2-[5H-imidazo[4,3-a]isoindol-5-yl]ethyl)cyclohexyl]sulfanyl]ethan-1-one: At room temperature, to a solution of 4-fluoro-4-(2-[5H-imidazo[4,3-a]isoindol-5-yl]ethyl)cyclohexyl methanesulfonate (1.6 g, 4.23 mmol) in DMF (20 mL) was added 1-(potassiosulfanyl)ethan-1-one (1.45 g, 12.72 mmol). The resulting mixture was stirred at 75° C. for 4 h. Then the reaction mixture was diluted with water (80 mL) and extracted with EtOAc (100 mL×2). The organic phases were combined, washed with brine and dried over Na₂SO₄. The solvent was removed under reduced pressure and the residue was purified by flash chromatography eluting with MeOH in DCM (1% to 5% gradient) to yield 1-[[4-fluoro-4-(2-[5H-imidazo[4,3-a]isoindol-5-yl]ethyl)cyclohexyl]sulfanyl]ethan-1-one (1.45 g, 81%) as light brown oil. MS: m/z=359.05 [M+H]⁺.

4-Fluoro-4-(2-[5H-imidazo[4,3-a]isoindol-5-yl]ethyl)cyclohexane-1-sulfonic acid: At 0° C., to a solution of 1-[[4-fluoro-4-(2-[5H-imidazo[4,3-a]isoindol-5-yl]ethyl)cyclohexyl]sulfanyl] ethan-1-one (1.45 g, 4.05 mmol) in HCOOH (20 mL) was added H₂O₂ (30%, 4 mL) dropwise. The resulting mixture was stirred at room temperature for 2 h. Then the reaction mixture was concentrated under reduced pressure and the residue was purified by reverse phase chromatography eluting with MeCN in water (5% to 30% gradient) to yield 4-fluoro-4-(2-[5H-imidazo[4,3-a]isoindol-5-yl]ethyl)cyclohexane-1-sulfonic acid (300 mg, 20%) as light yellow solid. MS: m/z=365.0 [M+H]⁺.

4-Fluoro-4-(2-[5H-imidazo[4,3-a]isoindol-5-yl]ethyl)cyclohexane-1-sulfonyl chloride: At 0° C., to a solution of 4-fluoro-4-(2-[5H-imidazo[4,3-a]isoindol-5-yl]ethyl)cyclohexane-1-sulfonic acid (300 mg, 0.82 mmol) in DCM (10 mL) was added (COCl)₂ (0.35 mL, 4.11 mmol) slowly, followed by the addition of one drop of anhydrous DMF. The resulting mixture was then stirred at room temperature for 1 h. After the reaction was done, the reaction mixture was concentrated under reduced pressure to yield 4-fluoro-4-(2-[5H-imidazo[4,3-a]isoindol-5-yl]ethyl)cyclohexane-1-sulfonyl chloride (310 mg, 98%) as light yellow solid which was used in the next step without further purification. MS: m/z=383.0 [M+H]⁺.

4-Fluoro-4-[2-[5H-imidazo[4,3-a]isoindol-5-yl]ethyl]cyclohexane-1-sulfonamide: At −78° C., a solution of 4-fluoro-4-(2-[5H-imidazo[4,3-a]isoindol-5-yl]ethyl)cyclohexane-1-sulfonyl chloride (310 mg, 0.81 mmol) in DCM (10 mL) was stirring while NH₃ gas was bubbled through it for 5 min. The resulting mixture was kept stirring while slowly warmed up to −40° C. over 20 min period. Then the reaction mixture was concentrated under reduced pressure and the residue was purified by prep-HPLC to give two pairs of enantiomeric products under the following conditions: XBridge BEH130 Prep C18 OBD Column, 19×150 mm, 5 μm; mobile phase, MeCN in water (with 0.05% TFA); 6% to 20% gradient in 20 min; Detector, UV 254/220 nm. Then four enantiomeric products were obtained by the further separation on chiral prep-HPLC under the following conditions: Phenomenex Lux 5p Cellulose-4, AXIA Packed, 21.2×250 mm, 5 μm; mobile phase, EtOH in hexane, 50% isocratic in 60 min; Detector, UV 254/220 nm.

Compound 121a: (25 mg, 8.4%, white solid, single stereoisomer), HPLC: 99.95% purity, RT=0.86 min. MS: m/z=364.15 [M+H]⁺. ¹H NMR (300 MHz, CD₃OD, ppm) δ=7.89 (s, 1H), 7.58 (d, J=7.5 Hz, 1H), 7.45 (d, J=7.5 Hz, 1H), 7.38 (t, J=7.2 Hz, 1H), 7.32-7.27 (m, 1H), 7.12 (s, 1H), 5.41 (t, J=4.8 Hz, 1H), 2.89-2.79 (m, 1H), 2.42-2.32 (m, 1H), 2.23-2.10 (m, 1H), 2.02-1.87 (m, 4H), 1.83-1.68 (m, 2H), 1.50-1.15 (m, 4H);

Compound 121b: (21 mg, 7.1%, white solid, single stereoisomer) HPLC: 97.9% purity, RT=0.88 min. MS: m/z=364.15 [M+H]⁺. ¹H NMR (300 MHz, CD₃OD, ppm) δ=7.95 (s, 1H), 7.59 (d, J=7.5 Hz, 1H), 7.47 (d, J=7.2 Hz, 1H), 7.39 (t, J=7.2 Hz, 1H), 7.33-7.28 (m, 1H), 7.15 (s, 1H), 5.43 (t, J=4.8 Hz, 1H), 2.98-2.92 (m, 1H), 2.40-2.28 (m, 1H), 2.21-1.86 (m, 5H), 1.70-1.55 (m, 4H), 1.40-1.23 (m, 2H);

Compound 121c: (26 mg, 8.8%, white solid, single stereoisomer), HPLC: 99.7% purity, RT=0.85 min. MS: m/z=364.1 [M+H]⁺. ¹H NMR (300 MHz, CD₃OD, ppm) δ=7.89 (s, 1H), 7.58 (d, J=7.5 Hz, 1H), 7.45 (d, J=7.5 Hz, 1H), 7.38 (t, J=7.2 Hz, 1H), 7.32-7.27 (m, 1H), 7.12 (s, 1H), 5.41 (t, J=4.8 Hz, 1H), 2.89-2.79 (m, 1H), 2.42-2.32 (m, 1H), 2.23-2.10 (m, 1H), 2.02-1.87 (m, 4H), 1.83-1.68 (m, 2H), 1.50-1.15 (m, 4H);

Compound 121d: (22 mg, 7.4%, white solid, single stereoisomer) HPLC: 99.0% purity, RT=1.66 min. MS: m/z=364.15 [M+H]⁺. ¹H NMR (300 MHz, CD₃OD, ppm) δ=7.95 (s, 1H), 7.59 (d, J=7.5 Hz, 1H), 7.47 (d, J=7.2 Hz, 1H), 7.39 (t, J=7.2 Hz, 1H), 7.33-7.28 (m, 1H), 7.15 (s, 1H), 5.43 (t, J=4.8 Hz, 1H), 2.98-2.92 (m, 1H), 2.40-2.28 (m, 1H), 2.21-1.86 (m, 5H), 1.70-1.55 (m, 4H), 1.40-1.23 (m, 2H).

### Example 2: Anti-Tumor Efficacy of Combination Therapy with M4112, a Small Molecule Inhibitor of IDO1 and TDO2, and Avelumab in Mouse Models of Colon Carcinoma

The studies evaluated the anti-tumor efficacy of combination therapy with the IDO1/TDO2 inhibitor M4112 and the anti-PD-L1 immunoglobulin (Ig)G1 monoclonal antibody avelumab in two mouse models of colon carcinoma, namely the CT26-KSA and MC38 tumor models. The combination therapy strongly inhibited tumor growth and moderately extended survival in both tumor models. These anti-tumor effects were greater than those of either therapy administered alone. Monotherapies resulted in only minor inhibition of tumor growth. Treatment with M4112 or avelumab alone resulted in partial inhibition of tumor growth in CT26-KSA (T/C=78% and 84%, respectively) and MC38 tumor-bearing mice (T/C=72% and 75%, respectively). Combination therapy with M4112 and avelumab resulted in stronger inhibition of tumor growth in both CT26-KSA (T/C=47%) and MC38 tumor-bearing mice (T/C=49%) relative to either treatment alone. The combination therapy also extended survival in both mouse models.

### Animals

BALB/C and C57BL/6 female mice were obtained from Charles River Laboratories. All mice used in these studies were 8-12 weeks of age.

TABLE 2
Doses in test

| | Dose | Dose | Dose | | Route of |
|---|---|---|---|---|---|
| | in | in μg/ | in | Volume | admini- |
| Test Article | mg/kg | mouse | mg/ml | in ml | stration |
| M4112 (solvent: 20% | 200 | 4000 | 40 | 0.1 | p.o. |
| Kleptose ® hydroxypropyl | | | | | |
| beta cyclodextrin (HPB) in | | | | | |
| 100 mM citrate buffer) | | | | | |
| *Avelumab (solvent: PBS) | 20 | 400 | 2 | 0.2 | i.v. |
| **Isotype control (non- | 20 | 400 | 2 | 0.2 | i.v. |
| targeting inactive anti-PD- | | | | | |
| L1 antibody; VL- | | | | | |
| A431G.D52E.R99Y, VH- | | | | | |
| wt) | | | | | |
| *M4112 vehicle: 20% | N/A | N/A | N/A | 0.1 | p.o. |
| Kleptose HPB (Parenteral | | | | | |
| Grade, Roquette) in 100 | | | | | |
| mM citrate buffer | | | | | |
| (Teknova, Cat # Q2445) | | | | | |

*For avelumab administration, dose in mg/kg is approximate, assuming a body weight of 20 g per mouse.
**Negative control.

### Statistical Analysis

Tumor growth data are presented in graphical form as the mean and standard error of the mean (SEM) of tumor volumes every 3 or 4 days. Two-way repeated measures analysis of variance (RM ANOVA) with Tukey's post-test was performed to assess differences in tumor volumes between treatment groups. Treatment/Control (T/C) values were calculated as the mean tumor volume of the treatment group divided by the mean tumor volume of the control group on the last day of observation and are expressed as percentages. In all cases, p<0.05 was considered statistically significant. Kaplan Meier survival curves were analyzed by Log-Rank (Mantel-Cox) test. A Bonferroni-corrected significance threshold of p<0.0125 was used to account for multiple comparisons.

### Example 2A: Anti-Tumor Efficacy of Combination Therapy with M4112 and Avelumab in CT26-KSA Tumor-Bearing Mice

The combination treatment of avelumab and M4112 in a CT26-KSA murine model was undertaken to evaluate the anti-tumor activity in BALB/c mice. The experimental and treatment conditions included: (1) M4112 vehicle (0.1 mL; p.o.; 2×/day for 3 weeks)+isotype control (400 μg; i.v.; Days 0, 3, 6); (2) M4112 vehicle (0.1 mL; p.o.; 2×/day for 3 weeks)+avelumab (400 μg; i.v.; Days 0, 3, 6); (3) M4112 (200 mg/kg; p.o.; 2×/day for 3 weeks)+isotype control (400 μg; i.v.; Days 0, 3, 6); and (4) M4112 (200 mg/kg; p.o.; 2×/day for 3 weeks)+avelumab (400 μg; i.v.; Days 0, 3, 6). The tumor volume, mice survival and mouse body weight were measured. Treatment with either avelumab or M4112 partially inhibited tumor growth relative to control treatment. Combination treatment with M4112 and avelumab resulted in greater inhibition of tumor growth than treatment with either agent alone. Survival was improved after combination therapy but not after treatment with M4112 or avelumab individually.

### Cell Lines

CT26-KS antigen (KSA, also known as EpCAM) colon carcinoma cells were generated as described previously (Xiang et al. 1997 Cancer Res. 57(21): 4948). Cells were tested and verified to be free of adventitious viruses and mycoplasma.

TABLE 3
Solutions for CT26-KSA cell culture

| Abbreviated name | Origin and contents |
|---|---|
| RPMI 1640 | Containing glutamine. |
| | Thermo Fisher Scientific |
| | (Gibco), Cat #11875093 |
| 10% Fetal bovine serum | Thermo Fisher Scientific |
| (FBS), heat-inactivated at | (Gibco), Cat # 16000-044 |
| 56° C. for 30 minutes | |
| L-glutamine | Thermo Fisher Scientific |
| | (Gibco), Cat # 25030081 |
| MEM non-essential amino | Thermo Fisher Scientific |
| acids (NEAA) | (Gibco), Cat # 11140050 |
| Na Pyruvate | Thermo Fisher Scientific |
| | (Gibco), Cat # 11360070 |
| MEM vitamin solution | Thermo Fisher Scientific |
| | (Gibco), Cat # 11120052 |
| Geneticin ® selective | Thermo Fisher Scientific |
| antibiotic | (Gibco), Cat # 10131035 |
| (G418 sulfate) | |
| PBS without Ca²⁺ or Mg²⁺ | Lonza (Biowhittaker), Cat # 17-516F |
| TrypLE Express | Thermo Fisher Scientific |
| | (Gibco), Cat # 12605010 |
| Trypan blue | Thermo Fisher Scientific |
| | (Gibco), Cat # 15250061 |

### Cell Culture

CT26-KSA cells were cultured in RPMI-1640 medium containing 10% FBS, 2 mM L-glutamine, 0.1 mM non-essential amino acids (NEAA), 1 mM Na Pyruvate, 1% MEM vitamin solution and 1% G418 antibiotic (1 mg/mL). All cells were maintained at 37′C and 5% CO₂ in aseptic conditions. Cells were passaged upon reaching 50-85% confluence for a total of 2 to 15 passages prior to in vivo implantation. Cells were harvested by trypsinization with TrypLE Express and viable cell counts were determined using a Countess or hematocrit chamber cell counter and trypan blue exclusion staining.

CT26-KSA Tumor Model BALB/c female mice were inoculated (subcutaneously (s.c.) in the right dorsal flank) with 1×10⁶ CT26-KSA cells in 0.1 mL sterile PBS. Treatment was initiated 12 days after tumor cell inoculation (Day 0), when tumors reached an average volume of approximately 150-200 mm³.

TABLE 4
Combination treatment with avelumab and M4112 in the
CT26-KSA murine model treatment groups
(N = 10 mice/group)

| Group | Treatment | Dose | | Route | Treatment Schedule |
|---|---|---|---|---|---|
| 1 | M4112 vehicle | 0.1 | mL | p.o. | 2x/day for 3 weeks |
| | Isotype control | 400 | μg | i.v. | Days 0, 3, 6 |
| 2 | M4112 vehicle | 0.1 | mL | p.o. | 2x/day for 3 weeks |
| | Avelumab | 400 | μg | i.v. | Days 0, 3, 6 |
| 3 | M4112 | 200 | mg/kg | p.o. | 2x/day for 3 weeks |
| | Isotype control | 400 | μg | i.v. | Days 0, 3, 6 |
| 4 | M4112 | 200 | mg/kg | p.o. | 2x/day for 3 weeks |
| | Avelumab | 400 | μg | i.v. | Days 0, 3, 6 |

The anti-tumor efficacy of combination therapy with M4112 and avelumab in an s.c. CT26-KSA colon carcinoma mouse model was investigated. CT26-KSA tumor-bearing mice were treated with M4112 (200 mg/kg, p.o., 2×/day for 3 weeks) and avelumab (400 μg/mouse; i.v., Days 0, 3, 6) in combination or as monotherapies. For the monotherapy conditions, M4112 was administered together with a non-binding, mutated anti-PD-L1 antibody (hereafter referred to as isotype control; 400 μg, i.v., Day 0, 3, 6), and avelumab was administered with the M4112 vehicle (0.1 mL, p.o., 2×/day for 3 weeks). As a control, mice were treated with the M4112 vehicle and the isotype control antibody.

Monotherapy with M4112 or avelumab partially inhibited CT26-KSA tumor growth relative to control treatment (T/C=78% and 84%, respectively, on Day 21), but only the M4112 monotherapy condition reached statistical significance (p=0.0251; see FIG. 3).

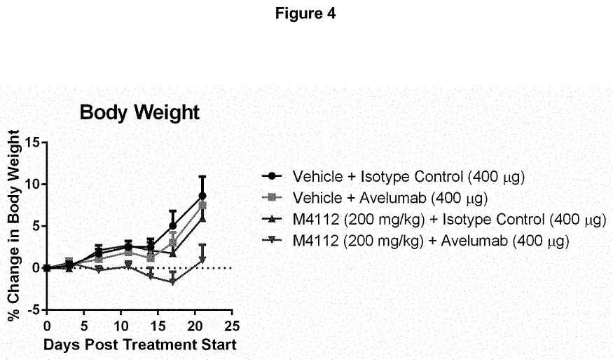

Combination therapy with M4112 and avelumab resulted in greater inhibition of tumor growth (T/C=47% on Day 21) compared with either M4112 or avelumab monotherapy (p=0.0007 and p<0.0001, respectively). Combination therapy with M4112 and avelumab also prolonged survival in CT26-KSA tumor-bearing mice relative to avelumab monotherapy (p=0.0104) and showed a trend towards prolonging survival relative to M4112 (see FIG. 3). Specifically, combination therapy resulted in a median survival time of 39.5 days, compared to 30.5 and 24 days for M4112 monotherapy and avelumab monotherapy, respectively. All treatments were well tolerated, as no major decreases in body weight were observed in mice in any of the treatment groups throughout the 21-day observation period (see FIG. 4).

### Example 2B: Anti-Tumor Efficacy of Combination Therapy with M4112 and Avelumab in MC38 Tumor-Bearing Mice

The combination treatment of avelumab and M4112 in the MC38 murine model was undertaken to evaluate the anti-tumor activity in C57BL/6 mice. The experimental and treatment conditions included: (1) M4112 vehicle (0.1 mL; p.o.; 2×/day for 4 weeks)+isotype control (400 μg; i.v.; Days 0, 2, 4); (2) M4112 vehicle (0.1 mL; p.o.; 2×/day for 4 weeks)+avelumab (400 μg; i.v.; Days 0, 2, 4); (3) M4112 (200 mg/kg; p.o.; 2×/day for 4 weeks)+isotype control (400 μg; i.v.; Days 0, 2, 4); and (4) M4112 (200 mg/kg; p.o.; 2×/day for 4 weeks)+avelumab (400 μg; i.v.; Days 0, 2, 4). The tumor volume, mice survival and mouse body weight were measured. Treatment with either avelumab or M4112 partially inhibited tumor growth relative to control treatment. Combination treatment with M4112 and avelumab resulted in greater inhibition of tumor growth than treatment with either agent alone.

### Cell Lines

MC38 colon carcinoma cells were obtained from Scripps Research Institute. Cells were tested and verified to be free of adventitious viruses and mycoplasma.

TABLE 5
Solutions for MC38 cell culture

| Abbreviated name | Origin and contents |
|---|---|
| Dulbecco's Modified Eagle | 1X, containing 4.5 g/L D-Glucose, |
| Medium (DMEM) | 2 mM glutamine, and |
| | 110 mg/L sodium pyruvate; |
| | Life Technologies, Cat # 11995-065 |
| 10% Fetal bovine serum | Life Technologies, Cat # 16000-044 |
| (FBS), heat-inactivated | |
| at 56° C. for 30 minutes | |
| 0.1 mM MEM non-essential | Life Technologies, Cat # 11140050 |
| amino acids (NEAA) | |
| Penicillin/Streptomycin | Thermo Fisher Scientific (Gibco), |
| | Cat # 15140-122 |
| Dulbecco's Phosphate | Life Technologies, Cat # 14190-144 |
| Buffered Saline 1X | |
| TrypLE Express plus | Life Technologies, Cat # 12605-010 |
| phenol red 1X | |
| Trypan blue | Thermo Fisher Scientific (Gibco), |
| | Cat # 15250061 |

### Cell Culture

MC38 cells were cultured in DMEM containing 4.5 g/L D-glucose, 2 mM glutamine, and 110 mg/L sodium pyruvate and supplemented with 10% FBS, 0.1 mM MEM NEAA, and penicillin/streptomycin. All cells were maintained at 37° C. and 5% CO₂ in aseptic conditions. Cells were passaged upon reaching 50-85% confluence for a total of 2 to 15 passages prior to in vivo implantation. Cells were harvested by trypsinization with TrypLE Express and viable cell counts were determined using a Countess or hematocrit chamber cell counter and trypan blue exclusion staining.

### MC38 Tumor Model

C57BL/6 female mice were inoculated (s.c. in the right dorsal flank) with 1×10⁶ MC38 cells in 0.1 mL sterile PBS. Treatment was initiated when tumors reached an average volume of approximately 50-100 mm³ (Day 0).

TABLE 6
Combination treatment with avelumab and
M4112 in the MC38 murine model
treatment groups (N = 10 mice/group)

| Group | Treatment | Dose | | Route | Treatment Schedule |
|---|---|---|---|---|---|
| 1 | M4112 vehicle | 0.1 | mL | p.o. | 2x/day for 4 weeks |
| | Isotype control | 400 | μg | i.v. | Days 0, 2, 4 |
| 2 | M4112 vehicle | 0.1 | mL | p.o. | 2x/day for 4 weeks |
| | Avelumab | 400 | μg | i.v. | Days 0, 2, 4 |
| 3 | M4112 | 200 | mg/kg | p.o. | 2x/day for 4 weeks |
| | Isotype control | 400 | pμg | i.v. | Days 0, 2, 4 |
| 4 | M4112 | 200 | mg/kg | p.o. | 2x/day for 4 weeks |
| | Avelumab | 400 | μg | i.v. | Days 0, 2, 4 |

The anti-tumor efficacy of combination therapy with M4112 and avelumab in an s.c. MC38 colon carcinoma mouse model was investigated. MC38 tumor-bearing mice were treated with M4112 (200 mg/kg, p.o., 2×/day for 4 weeks) and avelumab (400 μg/mouse; i.v., Days 0, 2, 4) in combination or as monotherapies. For the monotherapy conditions, M4112 was administered together with isotype control (400 μg, i.v., Day 0, 2, 4), and avelumab was administered with the M4112 vehicle (0.1 mL, p.o., 2×/day for 4 weeks). As a control, mice were treated with the M4112 vehicle and the isotype control antibody.

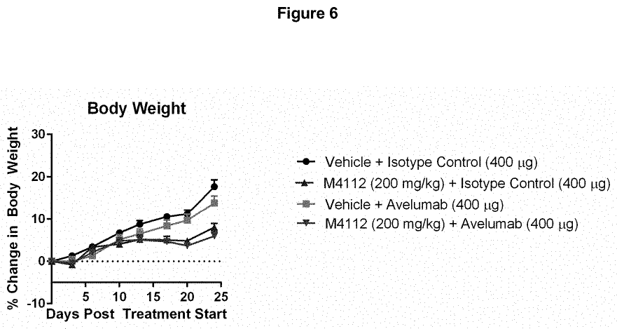

M4112 or avelumab monotherapy moderately inhibited MC38 tumor growth relative to control treatment (T/C=72% and p<0.0001; and T/C=75% and p=0.0003, respectively; Day 24; see FIG. 5). Combination therapy with M4112 and avelumab resulted in greater inhibition of tumor growth (T/C=49% on Day 24) compared with either M4112 or avelumab monotherapy (p=0.0008 and p<0.0001, respectively, Day 24). No major decreases in body weight were observed for any of the treatment groups throughout the 24-day observation period, suggesting treatments were well-tolerated (see FIG. 6).

### Example 3: Avelumab Dose Response in One-Way MLR Assay

This example describes the in vitro characterization of M4112, alone and in combination with the anti-tumor compound avelumab, using one-way Mixed Lymphocyte Reaction (MLR) assays. An MLR is a functional assay that measures the immune response (IFN production) of lymphocytes from one individual, the responder (PBMCs from one donor) to lymphocytes from another individual, the stimulator (mDCs from another donor). Using this functional assay, the immune IDO1 expressing cells (mDCs) were cultured with PBMCs in the presence or absence of the IDO1/TDO2 inhibitor, M4112, and the immune response was evaluated by looking at IFNγ production.

Unpurified human buffy coats, containing platelets and white blood cells, were purchased from Research Blood Components, LLC from donors KP33289 and 17988. Human PBMCs were isolated from the buffy coat of donor KP33289 and used in MLR assays. Human PBMCs and, subsequently imDCs, were isolated from the buffy coat of donor 17988. Mature DCs were derived from the imDCs and used in MLR assays.

A one-way MLR assay was performed with a 2.3:1 ratio of PBMC to mDC (stimulated with LPS) under two different conditions. In Experiment A, IDO-high mDCs (as measured by FACS) were mixed with PBMC, harvested on day 4 and treated with avelumab (solvent: 51 mg/mL D-mannitol, 0.6 mg/mL acetic acid, 0.5 mg/mL polysorbate 20, NaOH qs pH 5.2) at 10000, 2500, 635, 56.3, 39.1, 9.8, 2.4 or 0.61 ng/mL per well with either vehicle control (DMSO) or 5 μM of IDO1 inhibitor. In Experiment B, IDO-low mDCs (as measured by FACS) were mixed with PBMC, harvested on day 3 and treated under the same compound concentrations as described above. Both experiments measured the change in IFNγ concentration using the optical density of a standard curve from an ELISA.

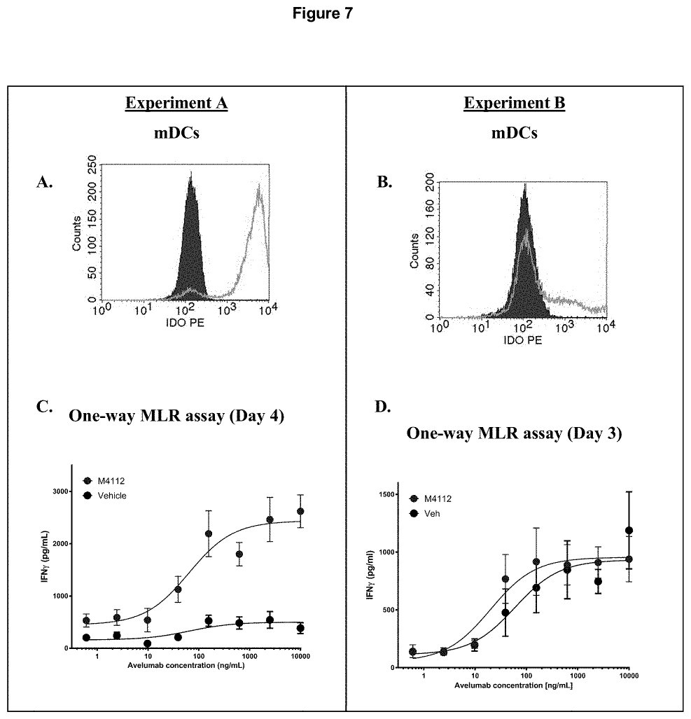

The results indicated that in high-IDO1 expressing conditions (Experiment A), the activity of avelumab was restored to normal levels with the addition of the IDO1 inhibitor (FIG. 7C), whereas at low-IDO1 expressing conditions (Experiment B), the addition of the IDO1 inhibitor resulted in no change in avelumab activity (FIG. 7D). Taken together, these data suggest that avelumab activity is suppressed in the presence of high levels of IDO1, but that the IDO1 inhibitor M4112 can reverse this decreased activity and cause an increase in IFNγ in an avelumab dose-dependent manner. IDO1 is therefore a useful biomarker for the selection of patients that receive and respond to M4112/avelumab combination therapy.

In summary, the results presented demonstrate that M4112 efficiently induces immune cell stimulation and rescues the suppressed activity of avelumab when used in combination therapy in patients with IDO1-high expressing cancers.

### Example 4: Combination Study with Anti-PD-L1 Antibody Avelumab and IDO1 Inhibitor M4112

Illustrated is an ongoing clinical phase I, open-label study designed to determine the safety, tolerability, pharmacokinetics (PK), pharmacodynamics (PD) and preliminary antitumor activity of M4112 as single agent (Part IA only) and in combination with avelumab (Part IB). The target population for Part I (Dose Escalation) comprises subjects with advanced or metastatic solid malignancies for whom no effective standard therapy exists or has failed, or subjects who are intolerant to established therapy known to provide clinical benefit for their condition.

Pre-specified ascending doses of M4112 as single agent in 28-day cycles are foreseen. Dose escalation decisions will be driven primarily by dose limiting toxicity (DLT), safety and tolerability. The maximal tolerated dose (MTD) may not be reached in the anticipated PD active dose range of M4112 as a targeted agent. Therefore, the assessment of PD effects will be used to explore the optimal biological dose (OBD) and to inform on the recommended phase 2 dose (RP2D), when the MTD cannot be determined.

In principle, dose escalation of single agent M4112 (Part IA) will proceed according to the recommendation of the Scottish Medicines Consortium (SMC) to at least the upper end of the above given dose range, unless the MTD has been reached or there is excess of PK nonlinearity, or the SMC recommends to end dose escalation following review of safety, tolerability, PK and PD results.

The starting dose of M4112 in combination with avelumab will be dependent on the observed safety, tolerability and PK/PD profile during dose escalation in the single agent cohort (Part IA) and will lag at least 1 dose level behind the last completed safe dose level of M4112 as single agent, which was confirmed as safe by the SMC. For selection of the starting dose, predictions based on an updated PK/PD model, including data from a combination therapy model, all available PK/PD data from completed cohorts of M4112 as single agent will be considered taking upregulated IDO and TDO activity by avelumab treatment into consideration. Dose escalation will proceed until the OBD of M4112 in combination with a fixed dose of avelumab is reached or dose escalation ends due to occurrence of DLTs establishing MTD.

Once the OBD or MTD of M4112 administered in combination with avelumab is estimated (dose finding portion), the dose expansion phase will be opened to further characterize the combination in term of safety profile, anti-tumor activity, PK, PD and biomarker modulation.

TABLE 7
Dose findings

| Arms | Assigned Interventions | |
|---|---|---|
| Dose finding | | |
| Part 1A | | |
| | Group 1: M4112 | 100 mg BID |
| | Group 2: M4112 | 200 mg BID |
| | Group 3: M4112 | 400 mg BID |
| | Group 4: M4112 | 600 mg BID |
| | Group 5: M4112 | 800 mg BID |
| | Group 6: M4112 | 900 mg BID |
| Part 1B | | |

Group 1: M4112 Dose level 1 BID: avelumab
10 mg/kg IV Q2W
Group 2: M4112 Dose level 2 BID: avelumab
10 mg/kg IV Q2W
Group 3: M4112 Dose level 3 BID: avelumab
10 mg/kg IV Q2W
Group 4: M4112 Dose level 4 BID: avelumab
10 mg/kg IV Q2W

Key inclusion criteria for Part I (dose escalation): Male or female subjects 18 years of age with histologically or cytologically proven advanced or metastatic solid malignancies for whom no effective standard therapy exists or has failed or subjects who are intolerant to established therapy known to provide clinical benefit for their condition (dose escalation cohorts; Part 1). An Eastern Cooperative Oncology Group (ECOG) performance status (PS) of 0 to 1 at screening and adequate hematological, renal and hepatic function as defined by protocol specified criteria.

Exclusion criteria: Intolerance to immune checkpoint inhibitor therapy as defined by the occurrence of an adverse drug reaction requiring drug discontinuation (dose escalation cohorts), concurrent anticancer treatment or immunosuppressive agents. Prior organ transplantation including allogeneic stem cell transplantation, brain metastases (except those meeting certain protocol specified criteria which are acceptable), significant acute or chronic infections, a history of cardiovascular/cerebrovascular disease or current significant cardiac conduction abnormalities and hypokalemia as specified in the protocol. Warfarin or other Vitamin K antagonists treatment, strong inhibitors or inducers of cytochrome P450 (CYP)3A4 and drugs with a narrow therapeutic index, which are predominantly metabolized by CYP3A4 and drugs known to have a high risk to prolong QTc as per label. Pregnancy or lactation. Severe hypersensitivity reactions to monoclonal antibodies, known hypersensitivity to the IMPs or to one or more of the excipients of M4112 or avelumab, autoimmune diseases (inflammatory bowel diseases, interstitial lung disease or pulmonary fibrosis) and live vaccines within 28 days prior to study entry.

Statistics: The number of patients to be enrolled in the Dose Finding Phase will depend on the observed safety profile and the number of tested dose levels. Up to approximately 24 patients for M4112 as single agent and 18 patients for combination with avelumab are projected to be enrolled in the study.

The Investigational Medicinal Product will be given as follows: M4112: Subjects will be administered an oral dose of M4112 BID (or a regimen determined by the SMC) at least 1 h prior to a meal and at least 2 h after a meal in 28-day cycles. In subjects receiving combination treatment, M4112 will be administered after premedication (within 30 min) and before infusion start of avelumab, as applicable. Avelumab (in combination with M4112): All subjects will receive intravenous infusion of avelumab 10 mg/kg over 1 h (−10 min/+20 min, i.e., over 50 to 80 min) every 2 weeks (on Day 1 and Day 15 of each cycle). The dose of avelumab will be calculated based on the weight of the subject determined within 72 h prior to the day of drug administration. The dose of avelumab used for the previous administration can be repeated if the change in the subject's weight is 10% or less than the weight used for the last dose calculation. In order to mitigate infusion-related reactions, subjects will receive a premedication regimen of 25 to 50 mg diphenhydramine and 500 to 650 mg paracetamol (acetaminophen, intravenous or oral equivalent) approximately 30 to 60 min prior to each dose of the first 4 infusions. Premedication should be administered for subsequent avelumab doses based upon clinical judgment and presence/severity of prior infusion reaction. This regimen may be modified based on local treatment standards and guidelines as appropriate, provided it does not include systemic corticosteroids.

Tumor assessment: Anti-tumor activity will be assessed by radiological tumor assessments at 8-week intervals, using RECIST version 1.1. Complete and partial responses will be confirmed on repeated imaging at least at 4 weeks after initial documentation. After 7 months from enrollment in the study, tumor assessments will be conducted less frequently, i.e., at 12-week intervals. In addition, radiological tumor assessments will also be conducted whenever disease progression is suspected. If radiologic imaging shows progressive disease, tumor assessment will be repeated at least >6 weeks later in order to confirm progressive disease if patient if continued to be treated through progressive disease.

Pharmacokinetic: PK parameters for M4112 will be based on plasma concentrations collected and assessed pre-dose and at multiple times post-dosing on Days 1 and 15 of Cycle 1. Pre-dose and 2 h post-dose on Day 1, Cycle 2 will also be assessed. For avelumab (Part IB), serum concentrations will be assessed at pre-dose and post-dose (end of infusion) collections on Day 1 of Cycles 1, and every 2nd cycle thereafter, with pre-dose samples on Day 15 on alternative cycles.

Biomarker Assessments: A key objective of the biomarker analyses that will be performed in this study is to investigate pharmacodynamic biomarkers that are predictive of dose for M4112 as single agent and in combination with avelumab. Assessments will include but not be limited to plasma Kyn and Trp and IDO1 activity in ex vivo stimulated whole blood. Furthermore, immune phenotyping of T cells, B cells, NK cells, leukocytes, and Tregs subsets in PBMCs and PD-1, PD-L1 and IDO1 expression on T cells (CD3, CD4 and CD8), NK cells, monocytes and dendritic cell populations will be investigated.

Peripheral Blood: In addition, peripheral blood specimens will be retained as whole blood, serum, or plasma in a biobank for exploratory biomarker assessments, unless prohibited by the patient or by decision of the Institutional Review Board or Ethics Committee. Samples may be used to identify or characterize cells, DNA, RNA, or protein markers known or suspected to be of relevance to the mechanisms of action, or the development of resistance to M4112+/−avelumab.

1. A method for treating an IDO1-positive cancer that induces an escape pathway to checkpoint inhibitor treatment in a subject in need thereof, comprising administering to the subject an anti-PD-L1 antibody, or an antigen-binding fragment thereof, and an IDO1 inhibitor, wherein the IDO1 inhibitor 3-(5-fluoro-1H-indol-3-yl)pyrrolididine-2,5-dione is excluded.

2. The method according to claim 1, wherein the anti-PD-L1 antibody mediates antibody-dependent cell-mediated cytotoxicity.

3. The method according to claim 1, wherein the anti-PD-L1 antibody comprises a heavy chain, which comprises three complementarity determining regions having amino acid sequences of SEQ ID NOs: 1, 2 and 3, and a light chain, which comprises three complementarity determining regions having amino acid sequences of SEQ ID NOs: 4, 5 and 6.

4. The method according to claim 1, wherein the anti-PD-L1 antibody comprises the heavy chain having amino acid sequences of SEQ ID NOs: 7 or 8 and the light chain having amino acid sequence of SEQ ID NO: 9.

5. The method according to claim 1, wherein the anti-PD-L1 antibody is avelumab.

6. The method according to claim 1, wherein the IDO1 inhibitor is a dual inhibitor of IDO1 and TDO2.

7. The method according to claim 1, wherein the IDO1 inhibitor is a compound of formula I: 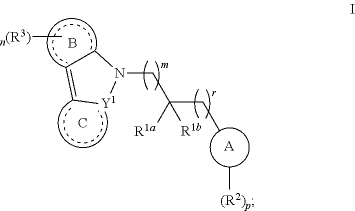
or a pharmaceutically acceptable salt thereof, wherein:
Y is CR or N;
Y¹ is C, CR, or N; wherein one of Y or Y is N;
R^{1a} is —R, halogen, -haloalkyl, -hydroxyalkyl, —OR, —SR, —CN, —NO₂, —SO₂R, —SOR, —C(O)R, —CO₂R, —C(O)N(R)₂, —NRC(O)R, —NRC(O)N(R)₂, —NRSO₂R, or —N(R)₂;
R^{1b} is —R, halogen, -haloalkyl, -hydroxyalkyl, —OR, —SR, —CN, —NO₂, —SO₂R, —SOR, —C(O)R, —CO₂R, —C(O)N(R)₂, —NRC(O)R, —NRC(O)N(R)₂, —NRSO₂R, or —N(R)₂;
or
R^{1a} and R^{1b}, together with the atom to which each is attached, may form a fused or spiro ring selected from C₅₋₁₀ aryl, a 3-8 membered saturated or partially unsaturated carbocyclic ring, a 3-7 membered heterocylic ring having 1-4 heteroatoms independently selected from nitrogen, oxygen, or sulfur, or a 5-6 membered monocyclic heteroaryl ring having 1-4 heteroatoms independently selected from nitrogen, oxygen, or sulfur; each of which is optionally substituted;
Ring A is C₅₋₁₀ aryl, a 3-8 membered saturated or partially unsaturated carbocyclic ring, a 3-7 membered heterocylic ring having 1-4 heteroatoms independently selected from nitrogen, oxygen, or sulfur, or a 5-6 membered monocyclic heteroaryl ring having 1-4 heteroatoms independently selected from nitrogen, oxygen, or sulfur;
each R² is independently —R, halogen, -haloalkyl, -hydroxyalkyl, —OR, —SR, —CN, —NO₂, —SO₂R, —SOR, —C(O)R, —CO₂R, —C(O)N(R)₂, —NRC(O)R, —NRC(O)N(R)₂, —NRSO₂R, or —N(R)₂;
Ring B is C₅₋₁₀ aryl, a 3-8 membered saturated or partially unsaturated carbocyclic ring, a 3-7 membered heterocylic ring having 1-3 heteroatoms independently selected from X¹, X², or X³, selected from nitrogen, oxygen, or sulfur, or a 5-6 membered monocyclic heteroaryl ring having 1-3 heteroatoms independently selected from X¹, X², or X³, each of which is selected from nitrogen, oxygen, or sulfur;
each R³ is independently —R, halogen, -haloalkyl, -hydroxyalkyl, —OR, —SR, —CN, NO₂, —SO₂R, —SOR, —C(O)R, —CO₂R, —C(O)N(R)₂, —NRC(O)R, —NRC(O)N(R)₂, —NRSO₂R, or —N(R)—;
Ring C is C₅₋₁₀ aryl, a 3-8 membered saturated or partially unsaturated carbocyclic ring, a 3-7 membered heterocylic ring having 1-4 heteroatoms independently selected from Z, Z¹, Z², Z³, or Z⁴, selected from nitrogen, oxygen, or sulfur, or a 5-6 membered monocyclic heteroaryl ring having 1-4 heteroatoms independently selected from Z, Z¹, Z², Z³, or Z⁴, each of which is selected from nitrogen, oxygen, or sulfur;
each R is independently hydrogen, C₁₋₆ aliphatic, C₃₋₁₀ aryl, a 3-8 membered saturated or partially unsaturated carbocyclic ring, a 3-7 membered heterocylic ring having 1-4 heteroatoms independently selected from nitrogen, oxygen, or sulfur, or a 5-6 membered monocyclic heteroaryl ring having 1-4 heteroatoms independently selected from nitrogen, oxygen, or sulfur; each of which is optionally substituted;
or
two R groups on the same atom are taken together with the atom to which they are attached to form a C₃₋₁₀ aryl, a 3-8 membered saturated or partially unsaturated carbocyclic ring, a 3-7 membered heterocylic ring having 1-4 heteroatoms independently selected from nitrogen, oxygen, or sulfur, or a 5-6 membered monocyclic heteroaryl ring having 1-4 heteroatoms independently selected from nitrogen, oxygen, or sulfur; each of which is optionally substituted;
m is 1 or 2;
n is 0, 1, 2, or 3;
p is 0, 1, 2, or 3; and
r is 0 or 1;
wherein when Ring A is non-fluoro substituted cyclohexyl, Ring B is benzo, and Ring C is 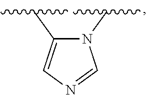 [SMILES: *c1cncn1*]
and R^{1a} is H, then R^{1b} cannot be OH.

8. The method according to claim 7, wherein Ring A is

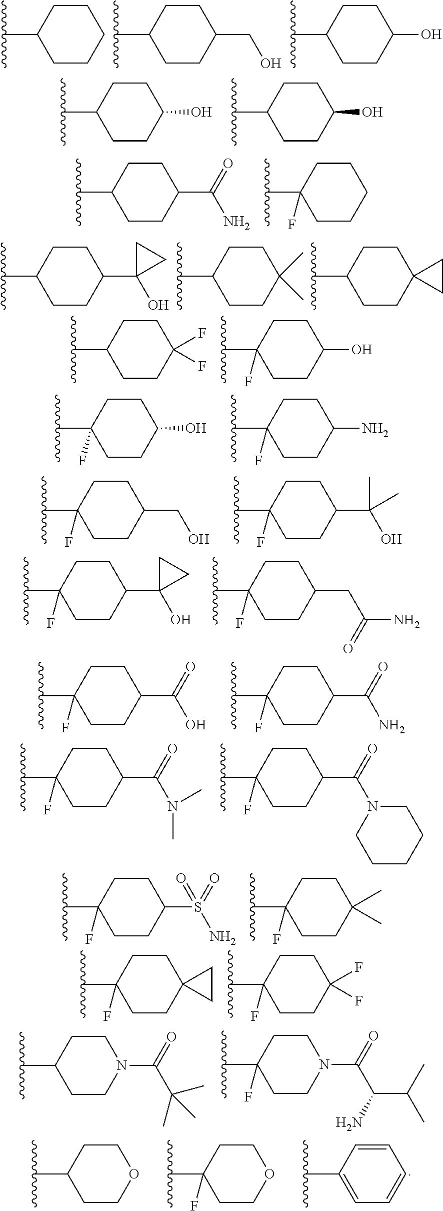

9. The method according to claim 7, wherein the IDO1 inhibitor is a compound of formula II: 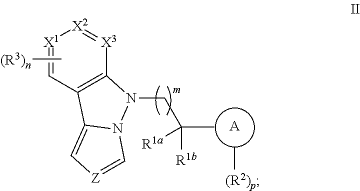
or a pharmaceutically acceptable salt thereof.

10. The method according to claim 7, wherein the IDO1 inhibitor is 4-fluoro-4-[2-[5H-imidazo[4,3-a]isoindol-5-yl]ethyl]cyclohexane-1-sulfonamide or c-4-fluoro-t-4-[(S)-2-(5H-imidazo[5,1-a]isoindol-5-yl)-ethyl]-cyclohexane-r-1-sulfonic acid amide, or a pharmaceutically acceptable salt thereof.

11. The method according to claim 1, wherein the IDO1-positive cancer shows an IDO1 expression that exceeds an IDO1 level predetermined prior to administering to the subject the anti-PD-L1 antibody and/or the IDO1 inhibitor.

12. The method according to claim 1, wherein the subject underwent at least one round of prior cancer therapy; wherein, optionally, the cancer was resistant or became resistant to prior therapy.

13. The method according to claim 1, wherein the cancer is a metastatic or locally advanced unresectable solid tumor.

14. The method according to claim 1, wherein the cancer is selected from malignant melanoma, acute myelogenous leukemia, pancreatic, colorectal, lung, prostate, cervical, brain, liver, head and neck, endometrial, esophageal, breast, and ovarian cancer.

15. (canceled)

16. (canceled)

17. The method according to claim 1, comprising the steps of: (a) determining that an IDO1 level in a cancer sample isolated from the subject exceeds an IDO1 level predetermined prior to the first receipt of the prior cancer therapy and/or the anti-PD-L1 antibody, and (b) administering the IDO1 inhibitor to the subject;
wherein the subject has received prior cancer therapy and/or the PD-L1 antibody prior to the first administration of the IDO1 inhibitor.

18. (canceled)

19. (canceled)

20. (canceled)

21. (canceled)

22. (canceled)

23. (canceled)

24. A method for predicting the likelihood that a subject suffering from an IDO1-positive cancer, which induces an escape pathway to checkpoint inhibitor treatment and is a candidate for treatment with an anti-PD-L1 antibody and an IDO1 inhibitor, wherein the IDO1 inhibitor 3-(5-fluoro-H-indol-3-yl)pyrrolididine-2,5-dione is excluded, will respond to the treatment, comprising determining the IDO1 expression by means of surrogate markers, which are levels of tryptophan, kynurenine or both, or a modification of a ratio of tryptophan and kynurenine, in a sample obtained from the subject, wherein a higher expression, as compared to a predetermined value, indicates that the subject is likely to respond to the treatment; and wherein, optionally, the surrogate marker is determined by means of LC/MS/MS.

25. The method according to claim 24, wherein the higher IDO1 expression correlates with an increase in kynurenine levels of at least 10%, 20%, 30%, 40% or 50%.

26. A method for monitoring the response to a treatment of a cancer which is mediated and/or propagated by IDO1 expression, and which induces an escape pathway to checkpoint inhibitor treatment, wherein a kynurenine plasma level is determined in a sample withdrawn from a subject with said cancer which is undergoing treatment with an anti-PD-L1 antibody and an IDO1 inhibitor, wherein the IDO1 inhibitor 3-(5-fluoro-1H-indol-3-yl)pyrrolididine-2,5-dione is excluded, and wherein a decrease in the kynurenine plasma level relative to a predetermined level indicates an increased likelihood that the subject responds to the treatment.

27. The method according to claim 26, wherein the decrease is at least 10%, 20%, 30%, 40% or 50%.

28. Method of monitoring the response to a treatment of a cancer which is mediated and/or propagated by IDO1 expression, and which induces an escape pathway to checkpoint inhibitor treatment, wherein an IDO1 plasma level is determined in a sample withdrawn from a subject with said cancer which is undergoing treatment with an anti-PD-L1 antibody and an IDO1 inhibitor, wherein the IDO1 inhibitor 3-(5-fluoro-1H-indol-3-yl)pyrrolididine-2,5-dione is excluded, and wherein a decrease in the IDO1 plasma level relative to a predetermined level indicates an increased likelihood that the subject responds to the treatment.
